(19) United States
(12) Patent Application Publication
(10) Pub. No.: US 2024/0002424 A1
(43) Pub. Date: Jan. 4, 2024

(54) HEXOSAMINE COMPOUNDS AND METHODS THEREOF

(71) Applicant: National Institute of Immunology, New Delhi (IN)

(72) Inventors: Srinivasa-Gopalan SAMPATHKUMAR, New Delhi (IN); Shubham PARASHAR, Kurukshetra Haryana (IN); Anam TASNEEM, Uttar Pradesh Aligarh (IN); Jyoti RAUTELA, Delhi (IN)

(73) Assignee: National Institute of Immunology, New Delhi (IN)

(21) Appl. No.: 18/248,503

(22) PCT Filed: Jun. 16, 2022
(86) PCT No.: PCT/IN2022/050551

§ 371 Date: Apr. 10, 2023

(30) Foreign Application Priority Data
Jun. 16, 2021 (IN) 202111026912

(51) Int. Cl. C07H 15/18; A61K 31/7028; C12N 5/00

(52) U.S. Cl. CPC C07H 15/18; A61K 31/7028; C12N 5/0006

(57) ABSTRACT

The present disclosure provides compounds of Formula I and a process of preparing the compounds of Formula I. The present disclosure further provides a compound of Formula II, Formula III, and Formula IV. The present disclosure provides compounds of Formula I that are capable of modifying cell-cell interactions, cell-pathogen interactions, or cell-extracellular matrix interactions, and methods thereof.

### FIELD OF INVENTION

The present invention relates in general, to the field of pharmaceutical compounds, particularly to the hexosamine compounds of Formula I and compositions comprising the same. The present invention further relates to a method of preparation of compounds of Formula I and compositions thereof. The invention also relates to use of said compounds of Formula I and pharmaceutical compositions which are capable of modulating/inhibiting cell-cell interactions, cell-pathogen interactions, or cell-extracellular matrix interactions.

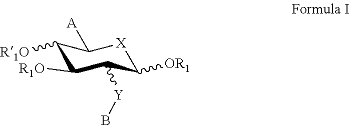

### BACKGROUND

Mammalian cell surface displays a dense layer of carbohydrate known as the glycocalyx which is composed of glycoproteins, proteoglycans, and glycolipids and are collectively known as glycoconjugates or glycans. These glycans govern cell-cell, cell-matrix, and cell-pathogen interactions. These glycans regulate biological processes such as fertilization, differentiation, migration, immune response, homeostasis, and can modulate many signaling pathways. Glycans primarily helps in structural and modulatory properties, including nutrient storage and sequestration, specific recognition by other molecules such as glycan-binding proteins (GBPs); and for molecular mimicry of host glycans. Modifying the glycan structures would hence directly impart its effect in the respective functional areas on the cell surface and the secretome. Therefore, modification of the glycans has become one of the extensive area of research to achieve towards development of drugs for various diseases.

An attractive way to modulate glycan structures is via the exploitation of biosynthetic pathways known as ‘metabolic glycan engineering’ (MGE) (Buettner et al., Front Immunol 9, 2485, 2018). MGE exploits the permissivity of biosynthetic pathways to process analogues of natural monosaccharides through the glycan biosynthetic pathways (Prescher and Bertozzi, Cell 126, 851-854.2006). Past research efforts have shown the application of MGE in varied areas for fluorescent imaging, glycan-tagging, and mass spectrometry. Based on the natural biosynthetic pathways, synthetic analogues of N-acetyl-D-mannosamine (ManNAc), N-acetyl-D-galactosamine (GalNAc), and N-acetyl-D-glucosamine (GlcNAc) have been employed, respectively, for the engineering of sialic acids, MTOG, and β-O-GlcNAc-ylation. Subsequent studies have revealed that GalNAc derivatives are more efficient compared to GlcNAc derivatives for engineering of β-O-GlcNAc due to metabolic equilibrium considerations.

The ability to express sialic acid analogues resulted in the advent of bio-orthogonal ligation on living animals and living cells. Pairs of reactive functional groups such as alkyne-azide, ketone-hydrazide, thiol-maleimide, alkene-tetrazine cycloaddition, inverse electron demand Diels-Alder reactions, and photochemical activation of azide and diazirine moieties have been developed. Particularly, the alkyne-azide cycloaddition is well exploited through the copper (I) assisted azidealkyne cycloaddition (CuAAC) and strain-promoted azide-alkyne cycloaddition (SPAAC) for the attachment of biochemical tags, such as biotin, FLAG-peptide, and fluorophores. In a complementary approach, few of the analogues carrying fluoro- and thiol-substituents were found to act as inhibitors of sialoglycans and MTOG, either with or without metabolic incorporation.

However, considering the diversity of glycan-protein and glycan-glycan interactions, the chemical space of N-acyl-hexosamine (HexNAc) analogues have not been explored sufficiently. Thus, there is a requirement for the findings of HexNAc analogues that are likely to fill in the void with interesting and useful properties for biomedicine and research applications.

Further, genetic methodologies, such as knock-out of specific glycosyl transferases or enzymes of complex carbohydrate metabolism, to modulate glycan-protein interactions are complex and result in severe phenotypes or show no effect due to redundancy of the enzymes involved in the glycan biosynthesis.

Based on the natural biosynthetic pathways, synthetic hexosamine analogues were studied towards modifying glycan structures and their effect on biosynthetic pathways. Hence, the small molecule hexosamine analogues are areas of great importance under biomedical research as they act as tools towards modulating the biosynthetic pathways in the cellular matrices. Thus, the identification and development of new small molecule compounds in treating or modifying various diseases or conditions associated with inhibition/modulation of the glycan biosynthetic pathways opens up novel pharmaceutical avenues.

### SUMMARY OF INVENTION

In first aspect of the present disclosure, there is provided a compound of Formula I

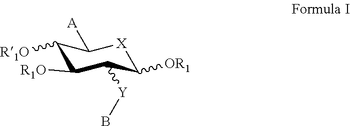

wherein A is C₁₋₁₂ alkyl, or —C₁₋₁₂ alkyl OR₁; wherein R₁ and R′₁ is independently selected from hydrogen, —C(O)C₁₋₁₂ alkyl, —C(O)C₂₋₁₂ alkenyl, or —C(O)C₂₋₁₂ alkynyl;
X is selected from O, S, Se, —CH₂, or —C(OH)R₂;
Y is —NR₂, or —CHR₂;
B is selected from hydrogen, —C(═Z)—(CH₂)ₘ—C₃₋₁₂ cycloalkyl, or —C(═Z)—(CH₂)ₘ—C₁₋₁₂ heterocyclyl;
Z is O, or CH₂;
R₂ is selected from hydrogen, C₁₋₁₂ alkyl, C₂₋₁₂ alkenyl, C₂₋₁₂ alkynyl, or C₆₋₁₄aryl;
m is 0 to 4;
provided when —(OR′₁) is equatorial, —(Y—B) is either axial or equatorial,
and m=0, then B≠—C(═Z)—(CH₂)ₘ—C₃ cycloalkyl.

In second aspect of the present disclosure, there is provided a compound of

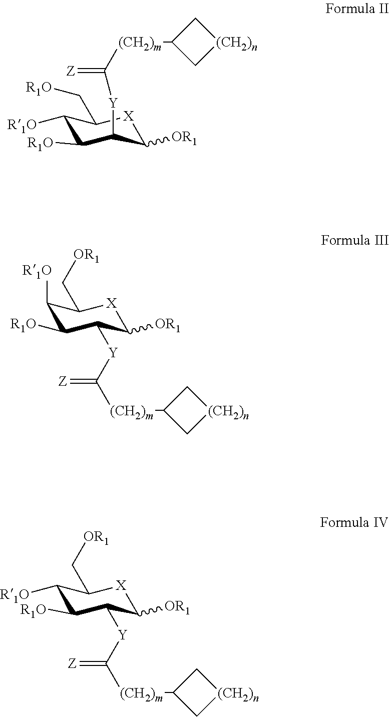

wherein R₁ and R′₁ is independently selected from hydrogen, or —C(O)C₁₋₃ alkyl; X is O; Y is —NR₂; Z is O; R₂ is hydrogen, or C₁₋₈ alkyl; m is 0 or 1,
n is 0 to 7;
when m of Formula II is 0, then n is 1 to 7;
when m of Formula IV is 0, then n is 1 to 7;
when m of Formula II is 1, then n is 0 to 7; and when m of Formula IV is 1, then n is 0 to 7.

In third aspect of the present disclosure, there is provided a process for preparing the compound of Formula I as disclosed herein, the process comprising reacting at least one selectively and orthogonally protected hexosamine salt of Formula A, with at least one carboxylic acid of Formula R₂CO₂H to obtain the compound of Formula I,

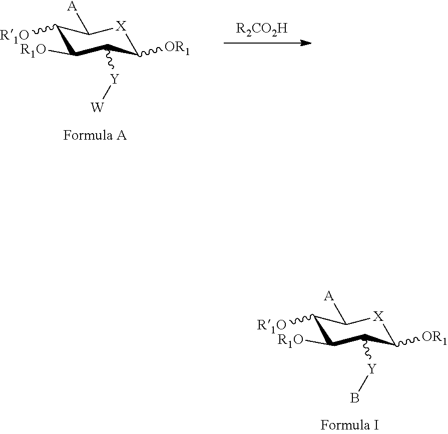

wherein R₂ is selected from C₃₋₁₂ cycloalkyl-(CH₂)ₘ—, C₁₋₁₂ heterocyclyl-(CH₂)ₘ, or C₂₋₂₄ alkyl heterocyclyl)-(CH₂)ₘ—;
W is selected from hydrogen, or C₁₋₁₂ alkyl;
A is C₁₋₁₂ alkyl, or —C₁₋₁₂ alkyl OR₁;
R₁ and R′₁ is independently selected from hydrogen —C(O)C₂₋₁₂ alkenyl, or —C(O)C₂₋₁₂ alkynyl;
X is selected from O, S, Se, —CH₂, or —C(OH)R₂,
Y is —NR₂, or —CHR₂;
B is selected from hydrogen, —C(═Z)—(CH₂)ₘ—C₃₋₁₂ cycloalkyl, or —C(═Z)—(CH₂)ₘ—C₁₋₁₂ heterocyclyl;
Z is O, or CH₂;
R₂ is selected from hydrogen, C₁₋₁₂ alkyl, C₂₋₁₂ alkenyl, C₂₋₁₂ alkynyl, or C₆₋₁₄aryl;
m is 0 to 7;
provided when —(OR′₁) is equatorial, —(Y—B) is either axial or equatorial,
and m=0, then B≠C(═Z)—(CH₂)ₘ—C₃ cycloalkyl.

In fourth aspect of the present disclosure, there is provided a pharmaceutical composition comprising the compound of Formula I optionally with at least one pharmaceutically acceptable salt thereof.

In fifth aspect of the present disclosure, there is provided a compound of Formula I capable of modifying cell-cell interactions, cell-pathogen interactions, or cell-extracellular matrix interactions, glycan-protein interactions, glycan-glycan interactions, sialic acids-proteins interactions, plant-pathogen interactions, sialyl-Lewis X/Lewis Y-selectins interactions, sialoglycoconjugates-siglecs interactions, polysialic acid-sialo-lectins interactions, sialic acid-neuraminidases interactions, sialic acid-hemagglutinin interactions, sialic acid-microbial adhesin interactions, or combinations thereof, wherein the compound is compound 1

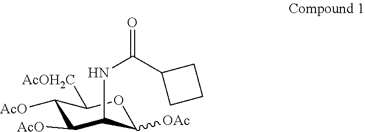

In sixth aspect of the present disclosure, there is provided a method for engineering cell surface epitopes, the method comprising: contacting a cell with a compound of Formula I or the pharmaceutical composition comprising the compound of Formula I optionally with at least one pharmaceutically acceptable salt thereof.

In seventh aspect of the present disclosure, there is provided a method for engineering of glycans on secreted glycoproteins in a mammalian bio-reactor system, in terms of biopharmaceuticals and biologics, the method comprising: contacting a cell with a compound of Formula I or the pharmaceutical composition comprising the compound of Formula I optionally with at least one pharmaceutically acceptable salt thereof.

These and other features, aspects, and advantages of the claimed subject matter will become better understood with reference to the following description. This summary is provided to introduce a selection of concepts in a simplified form. This summary is not intended to identify key features or essential features of the claimed subject matter, nor is it intended to be used to limit the scope of the subject matter.

### BRIEF DESCRIPTION OF DRAWINGS

FIG. 1 depicts the compounds of present invention (Formula II) in modulating the biosynthetic pathways, in an accordance with implementation of the present invention.

FIG. 2 depicts the compounds of present invention (Formula III) in modulating the biosynthesis of mucin-type O-glycans through the GalNAc salvage pathway, in an accordance with implementation of the present invention.

FIG. 3 illustrates the compounds of present invention (Formula IV) in engineering of β-O-GlcNAc-ylation of nuclear and cytoplasmic proteins, in an accordance with implementation of the present invention.

FIG. 4 depicts the flow cytometry studies for the measurement of cell surface of CD15s (sialyl-Lewis-X; sLeX) by treating with compounds of the present invention, (a) effect of structure of ManNAc compounds on sLeX expression; (b) the dose dependency of sLeX expression; (c) relative densities of HL-60 cells incubated with MA-1, MA-2, and MA-3 for 24 h as a measure of cytotoxicity; and (d) time course of sLeXexpression upon incubation with vehicle, MA-1, MA-2, and MA-3, in an accordance with implementation of the present invention.

FIG. 5 illustrates competitive processing of compounds of the present invention (ManNAc compounds), in accordance with an implementation of the present invention.

FIG. 6 illustrates A) Schematic illustration of sialic acids on the cell surface showing expression of wild-type NeuAc (pink diamonds), NeuAz (green diamonds) upon treatment with Ac4ManNAz (1c), and NeuCb (grey diamonds) upon treatment with Ac4ManNCb (1b); (B) Estimation of NeuAz expression via SPAAC using DBCO-Cy5 in Jurkat cells upon incubation with U, D, 1a, 1b, 1c, or 1d separately (50 μM, 24 h) and in combination 1d+1c, 1a+1c, or 1b+1c (50 μM each, 24 h); (C) Comparison of NeuAz levels upon either simultaneous addition of 1a+1c or 1b+1c (50 μM each) (s) or addition of 1a or 1b (50 μM) first, followed by the addition of 1c (50 μM) at 12 h (d) and estimation at 48 h; (D) effect on NeuAz expression by the ManNAc analogues 1a and 1b and GlcNAc analogues 2a and 2b upon co-incubation with 1c (50 μM each, 24 h); (E) effect on NeuAz and GalNAz expression on the cell surface upon incubation, respectively, with 1c alone, 1a+1c or 1b+1c, and incubation with 3 alone, 1a+3, or 1b+3 (50 μM each, 24 h), in accordance with an implementation of the present invention.

FIG. 7 illustrates representative histograms showing fluorescent intensity levels in cells incubated for 24 h either alone or in combination as depicted followed by DBCO-Cy5 reaction; B) fluorescent intensity levels in cells incubated for 24 h either alone or in combination with 1a as depicted followed by DBCO-Cy5 reaction; and C)) fluorescent intensity levels in cells incubated either alone or in combination with 3 as depicted, in accordance with an implementation of the present invention.

FIG. 8 illustrates (A) Flow cytometry estimation of NeuAz expression via SPAAC using Cy5-DBCO in HL-60 (human acute myeloid leukemia) cells upon incubation with either 1c (50 μM, 24 h) alone or 1c in combination with 1a, 1b, 1d, 1e, 1f, or 1g (50 μM each, 24 h). (B) Flow cytometry estimation of expression of LeX and sLeX epitopes on the surface, respectively, using anti-CD15 (HI98) and anti-CD15s (CSLEX1), in HL-60 cells incubated with D, 1a, 1b, 1c, 1d, 1e, 1f, or 1g (50 μM, 24 h). (C) Time course of surface expression of CD15s (CSLEX1) epitopes in HL-60 cells incubated with D, 1b, 1d, or 1g (50 μM) by flow cytometry. (D) Dual western blots probed using antiCD15s (CSLEX1) (upper green bands) and anti-3-actin (lower red bands; loading control) of total lysates of HL-60 cells incubated with D, 1d, 1e, 1f, 1g, 1a, or 1b (50 μM, 72 h); M, molecular weight markers. Flow cytometry estimation of (E) binding of E-selectin (CD62E-Fc) chimera protein and (F) anti-cutaneous lymphocyte antigen (CLA) (HECA452) antibody to HL-60 cells incubated with D, 1a, 1b, or 1d (50 μM, 24 h) (along with controls, viz., X, unstained; 2°, secondary only; IC, isotype control; U, untreated), in accordance with an implementation of the present invention.

FIG. 9 illustrates effect of HexNAc compounds in sialic acid content and cell density A) bar graph showing the levels of total sialic acids and glycoside bound sialic acids in HL-60 cells incubated with the HexNAc compounds as measured by the periodate-resorcinol assay; and B) bar graph showing the densities of HL-60 cells incubated with HexNAc compounds, in accordance with an implementation of the present invention.

FIG. 10 illustrates estimation of sialyl-lewis X epitopes A) histograms showing the fluorescence intensities of HL-60 cells stained with anti-CD15s (CSLEX1) antibody followed by Alexafluor488-conjugated secondary antibody; B) bar graphs showing the fluorescent intensity in HL-60 and Jurkat cells along with controls; and C) titration of saturation of CSLEX1 binding to HL-60 cells incubated with CSLEX1 antibody, in accordance with an implementation of the present invention.

FIG. 11 illustrates A) histograms and B) bar graphs showing the fluorescence intensities of HL-60 cells stained with human-E-selectin-Fc or mouse-E-selectin Fc chimera proteins followed by secondary antibody, C) histograms and D) graphs showing the fluorescence intensities of HL-60 cells stained with mCD62Fc, in accordance with an implementation of the present invention.

FIG. 12 illustrates modulation of sialoglycans by ManNAc analogues by far western blots of total lysates of HL-60 cells incubated with 1a, 1b, or 1d (50 μM, 72 h) (along with controls—untreated (U) and DMSO (D)) probed using biotinylated (A) Maackia amurensis lectin-II (MAL-II) and (B) Sambucus nigra agglutinin (SNA) followed by horse radish peroxidase conjugated avidin (HRP-avidin); 3-actin blots are shown as loading controls; flow cytometry estimation of surface epitopes of (C) MAL-II and (D) SNA epitopes in HL-60 cells treated with 1a, 1b, or 1d (50 μM, 24 h), along with controls (X, unstained; A, HRP-avidin alone; U, untreated) probed using biotinylated lectins and FITC-conjugated avidin. (E) Enumeration of number of HL-60 cells adhered to surfaces, coated with (i) protein-G/BSA alone, (ii) protein-G/BSA and E-selectin-Fc chimera, or (iii) protein-G/BSA and L-selectin-Fc-chimera, after incubation with 1a-1g, along with untreated and vehicle treated control, in accordance with an implementation of the present invention.

FIG. 13 illustrates treatment with ManNAc analogues modulating adhesion to selectins, A) HL-60 cells treated with compounds and allowed to adhere on plastic surfaces pre-coated with mouse E-selectin-Fc/protein-G/BSA; B) mouse L-selectin/protein-G/BSA; and C) control (protein-G/BSA), in accordance with an implementation of the present invention.

### DETAILED DESCRIPTION

Those skilled in the art will be aware that the present disclosure is subject to variations and modifications other than those specifically described. It is to be understood that the present disclosure includes all such variations and modifications. The disclosure also includes all such steps, features, compositions and compounds referred to or indicated in this specification, individually or collectively, and any and all combinations of any or more of such steps or features.

### Definitions

For convenience, before further description of the present disclosure, certain terms employed in the specification, and examples are collected here. These definitions should be read in the light of the remainder of the disclosure and understood as by a person of skill in the art. The terms used herein have the meanings recognized and known to those of skill in the art, however, for convenience and completeness, particular terms and their meanings are set forth below.

The articles “a”, “an” and “the” are used to refer to one or to more than one (i.e., to at least one) of the grammatical object of the article.

The terms “comprise” and “comprising” are used in the inclusive, open sense, meaning that additional elements may be included. Throughout this specification, unless the context requires otherwise the word “comprise”, and variations, such as “comprises” and “comprising”, will be understood to imply the inclusion of a stated element or step or group of element or steps but not the exclusion of any other element or step or group of element or steps.

The term “including” is used to mean “including but not limited to”. “Including” and “including but not limited to” are used interchangeably.

In the structural formulae given herein and throughout the present disclosure, the following terms have been indicated meaning, unless specifically stated otherwise.

Furthermore, the compound of Formula (I) can be its derivatives, analogs, tautomeric forms, enantiomers, diastereomers, geometrical isomers, polymorphs, solvates, intermediates, metabolites, prodrugs or pharmaceutically acceptable salts and compositions.

The compounds described herein may contain one or more chiral centers and/or double bonds and therefore, may exist as stereoisomers, anomers, such as double-bond isomers (i.e., geometric isomers), regioisomers, enantiomers or diastereomers. Accordingly, the chemical structures depicted herein encompass all possible enantiomers and stereoisomers of the illustrated or identified compounds including the stereoisomerically pure form (e.g., geometrically pure, enantiomerically pure or diastereomerically pure) and enantiomeric and stereoisomeric mixtures. Enantiomeric and stereoisomeric mixtures can be resolved into their component enantiomers or stereoisomers using separation techniques or chiral synthesis techniques well known to the person skilled in the art. The compounds may also exist in several tautomeric forms including the enol form, the keto form and mixtures thereof. Accordingly, the chemical structures depicted herein encompass all possible tautomeric forms of the illustrated or identified compounds. It is also understood that some isomeric form such as diastereomers, enantiomers and geometrical isomers can be separated by physical and/or chemical methods and by those skilled in the art. Pharmaceutically acceptable solvates may be hydrates or comprising of other solvents of crystallization such as alcohols, ether, and the like.

According to the present invention, the compounds provided herein, includes all of the corresponding enantiomers and stereoisomers, that is, the pure form of the stereoisomers, in terms of geometrical isomers, anomers, enantiomers, or diastereomers, and the mixture of enantiomeric and stereoisomeric form of said compounds. Further, the mixture of enantiomeric and stereoisomeric form can be resolved into their pure components by the methods known in the art, such as chiral-phase gas chromatography, chiral-phase high performance liquid chromatography, crystallization, using chiral derivatizing agents etc. Also, the pure enantiomers and stereoisomers can be obtained from intermediates or metabolites and reagents that are in the form of pure enantiomers and stereoisomers by known asymmetric synthetic methods.

The term “pharmaceutically acceptable” refers to compounds or compositions that are physiologically tolerable and do not typically produce allergic or similar untoward reaction, including but not limited to gastric upset or dizziness when administered to subjects.

Pharmaceutically acceptable salts forming part of this invention include salts derived from inorganic bases such as Li, Na, K, Ca, Mg, Fe, Cu, Zn and Mn and ammonium, substituted ammonium salts, aluminum salts and the like; salts of organic bases such as triethylamine, choline, trialkylamine, pyridine, and the like, salts also include amino acid salts such as glycine, alanine, cystine, cysteine, lysine, arginine, phenylalanine, guanidine etc. Salts may include acid addition salts where appropriate which are sulphates, nitrates, phosphates, perchlorates, borates, hydrohalides, acetates, tartrates, maleates, fumarates, citrates, succinates, lactates, ascorbate, palmitate, oleate, pyruvate, pamoate, malonate, laurate, glutarate, glutamate, estolate, mesylates, trifluoroacetates, acetates, besylates, propionates, mandelates, hydrobromides, hydrochlorides, palmoates, methanesulphonates, tosylates, benzoates, salicylates, hydroxynaphthoates, benzenesulfonates, ascorbates, glycerophosphates, ketoglutarates and the like.

As used herein, the term “substituted” is contemplated to include all permissible substituents of organic compounds. In a broad aspect, the permissible substituents include acyclic and cyclic, branched and unbranched, carbocyclic and heterocyclic, aromatic and nonaromatic substituents of organic compounds. Illustrative substituents, for example, include those described herein above. The permissible substituents can be one or more and the same or different for appropriate organic compounds. For purposes of this disclosure, the heteroatoms such as nitrogen may have hydrogen substituents, and/or any permissible substituents of organic compounds described herein which satisfy the valences of the heteroatoms.

The term “alkyl” refers to straight or branched aliphatic hydrocarbon groups having one to twelve carbon atoms, which are attached to the rest of the molecule by a single atom, which may be optionally substituted by one or more substituents. Preferred alkyl groups include, without limitation, methyl, ethyl, n-propyl, isopropyl, butyl, isobutyl, t-butyl, pentyl, hexyl, heptyl, octyl and the like.

The term “alkenyl” refers to an aliphatic hydrocarbon group containing a carbon-carbon double bond and which may be straight or branched chain having 2 to 12 carbon atoms, which may be optionally substituted by one or more substituents. Preferred alkenyl groups include, without limitation, ethenyl, 1-propenyl, 2-propenyl, iso-propenyl, 2-methyl-1-propenyl, 1-butenyl, 2-butenyl and the like including poly-unsaturated fatty acids (PUFA).

The term “alkynyl” refers to a straight or branched hydrocarbyl radicals having at least one carbon-carbon triple bond and having 2-12 carbon atoms, which may be optionally substituted by one or more substituents. Preferred alkynyl groups include, without limitation, ethynyl, propynyl, butynyl and the like.

The term “cycloalkyl” refers to mono or polycyclic ring system with specified number of carbon atoms, which may be optionally substituted by one or more substituents and may be saturated, partially saturated or unsaturated. The polycyclic ring denotes hydrocarbon systems containing two or more ring systems with one or more ring carbon atoms in common, i.e., a spiro, fused or bridged structures. A cycloalkyl group may include zero bridgehead carbon atoms or one or two or more bridgehead carbon atoms. Thus, a cycloalkyl may be monocyclic, bicyclic, or polycyclic, depending upon the number of bridgehead and bridging carbon atoms.

The term “unbridged cycloalkyl” refers to the cycloalkyl groups with 3 to 12 carbon atoms as defined above without any bridgehead carbon atoms. Preferred unbridged cycloalkyl groups include, without limitation, cyclopropyl, cyclobutyl, cyclopentyl, cyclohexyl, cycloheptyl, cyclooctanyl, and the like. The term “bridged cycloalkyl” refers to the cycloalkyl groups with 4 to 12 carbon atoms as defined above with one or more bridgehead carbon atoms. Typical bridged cycloalkyls include, but are not limited to, adamantyl, adamantylmethyl, noradamantyl, camphanyl, bicyclo[1.1.0]butanyl, norboranyl (bicyclo[2.2.1]heptanyl), norbornenyl (bicyclo[2.2.1]heptanyl), norbornadienyl (bicyclo[2.2.1]heptadienyl), tricyclo[2.2.1.0]heptanyl, bicyclo[3.2.1]octanyl, bicyclo[3.2.1]octanyl, bicyclo[3.2.1]octadienyl, bicyclo[2.2.2]octanyl, bicyclo[2.2.2]octenyl, bicycl0[2.2.2]octadienyl, bicyclo[5.2.0]nonanyl, bicyclo[4.3.2]undecanyl, tricyclo[5.3.1.1]dodecanyl, and the like.

The term “aryl” refers to aromatic radicals having 6 to 14 carbon atoms, which may be optionally substituted by one or more substituents. Preferred aryl groups include not limited to phenyl, naphthyl, and the like.

The term “heterocyclyl” refers to a heterocyclic ring radical which may be optionally substituted by one or more substituents. The heterocyclyl ring radical may be attached to the main structure at any heteroatom or carbon atom that results in the creation of a stable structure.

Furthermore, the term “heterocyclyl” refers to a stable 1 to 12 membered rings radical, which consists of carbon atoms and heteroatoms selected from nitrogen, phosphorus, oxygen and sulfur. For purposes of this invention the heterocyclic ring radical may be monocyclic, bicyclic or tricyclic ring systems, and the nitrogen, phosphorus, carbon, or sulfur atoms in the heterocyclic ring radical may be optionally oxidized to various oxidation states. In addition, the nitrogen atom may be optionally quaternized; and the ring radical may be saturated, partially saturated or unsaturated. The term “heterocyclyl” refers to monocyclic or polycyclic ring, polycyclic ring system refers to a ring system containing 2 or more rings, preferably bicyclic or tricyclic rings, in which rings can be fused, bridged or spiro rings or any combinations thereof. A fused ring as used herein means that the two rings are linked to each other through two adjacent ring atoms common to both rings. The fused ring can contain one or more hetero atoms independently selected from N, P, O, or S. The rings can be either fused by one or more heteroatom or —CH— group. A bridged heterocyclyl ring as used herein means the heterocyclyl ring has one or more bridgehead atoms. The bridgehead atoms may be either carbon or heteroatoms.

Preferred heterocyclyl groups include, without limitation, azetidinyl, acridinyl, benzodioxolyl, benzodioxanyl, benzofuranyl, carbazolyl, cinnolinyl, dioxolanyl, indolizinyl, naphthyridinyl, perhydroazepinyl, phenazinyl, phenothiazinyl, phenoxazinyl, phthalazinyl, pyridyl, pteridinyl, purinyl, quinazolinyl, qunioxalinyl, quinolinyl, isoquinolinyl, tetrazolyl, imidazolyl, tetrahydroisoquinolinyl, piperidinyl, piperazinyl, homopiperazinyl, 2-oxoazepinyl, azepinyl, pyrrolyl, 4-piperidonyl, pyrrolidinyl, pyrazinyl, pyrimidinyl, pyridazinyl, oxazolyl, oxazolinyl, triazolyl, indanyl, isoxazolyl, isoxazolidinyl, thiazolyl, thiazolinyl, thiazolidinyl, isothiazolyl, quinuclidinyl, isothiazolidinyl, indolyl, isoindolyl, indolinyl, isoindolinyl, octahydroindolyl, octahydroisoindolyl, quinolyl, isoquinolyl, decahydroisoquinolyl, benzimidazolyl, thiadiazolyl, benzopyranyl, benzothiazolyl, benzooxazolyl, thienyl, morpholinyl, thiomorpholinyl, thiamorpholinyl sulfoxide, furyl, tetrahydrofuryl, tetrahydropyranyl, and the like.

The term “alkylaryl” refers to an aryl group directly bonded to an alkyl group, which may be optionally substituted by one or more substituents. For the purpose of the present disclosure, the arylalkyl group of the present disclosure refers to compounds with carbon atoms ranging between 7 to 26, which includes alkyl group with 1 to 12 carbon atoms and aryl ring with 6 to 14 carbon atoms. Preferred alkylaryl groups include, without limitation, —CH₂-phenyl, —C₂H₄-phenyl, —C₃H₆-phenyl, and the like.

The term “arylalkyl” refers to an aryl group directly bonded to an alkyl group, which may be optionally substituted by one or more substituents. For the purpose of the present disclosure, the arylalkyl group of the present disclosure refers to compounds with carbon atoms ranging between 7 to 26, which includes the aryl ring with 6 to 14 carbon atoms and alkyl group with 1 to 12 carbon atoms. Preferred arylalkyl groups include, without limitation, —C₆H₅—CH₂—, —C₆H₅—C₂H₄— and the like.

It is understood that included in the family of compounds of Formula (I) are isomeric forms including diastereomers, anomers, enantiomers, tautomers, and geometrical isomers in “E” or “Z” configurational isomer or a mixture of ‘E’ and ‘Z’ isomers. It is also understood that some isomeric form such as diastereomers, enantiomers and geometrical isomers can be separated by physical and/or chemical methods and by those skilled in the art.

Compounds disclosed herein may exist as single stereoisomers, anomers, and or mixtures of enantiomers and/or diastereomers. All such single stereoisomers, and mixtures thereof are intended to be within the scope of the subject matter described.

Compounds disclosed herein include isotopes of hydrogen, carbon, oxygen, fluorine, chlorine, iodine and sulfur which can be incorporated into the compounds, such as not limited to ²H (D), ³H (T), ¹¹C, ¹²C, ¹³C, ¹⁴C, ¹⁵N, ¹⁸F, ³⁵S, ³⁶Cl and ¹²⁵I. Compounds of this invention wherein atoms were isotopically labeled for example radioisotopes such as ³H, ¹³C, ¹⁴C, and the like can be used in metabolic studies, kinetic studies and imaging techniques such as positron emission tomography used in understanding the tissue distribution of the drugs. Compounds of the invention where hydrogen is replaced with deuterium may improve the metabolic stability and pharmacokinetics properties of the drug such as in vivo half-life. Compounds of the invention where isotopically labeled ¹⁸F can be useful as PET imaging studies.

The compounds described herein can also be prepared in any solid or liquid physical form, for example the compound can be in a crystalline form, in amorphous form and have any particle size. Furthermore, the compound particles may be micronized or nanonized, or may be agglomerated, particulate granules, powders, oils, oily suspensions or any other form of solid or liquid physical forms.

The compounds described herein may also exhibit polymorphism. This invention further includes different polymorphs of the compounds of the present invention. The term polymorph refers to a particular crystalline state of a substance, having particular physical properties such as X-ray diffraction, IR spectra, melting point and the like.

The term “hexosamine” refers to amino sugars which are obtained by substitution of hydroxyl groups with amines to a hexose selected from stereoisomers of aldohexoses or ketohexoses. The term “hexosamine compounds” refers to compounds comprising the hexosamines as the skeletal structure and which are modified on the ring structure, or on the side chain substitutions, or both. In the present disclosure, the hexosamine compounds include but not limited to compounds of mannosamines, galactosamines, or glucosamines. The compounds of Formula I are the hexasoamine derivatives or analogues in particular are mannosamine analogues (ManNAc compounds or derivatives or analogues), galactosamine analogues (GalNAc compounds or derivatives or analogues) or Glucosamine analogues (GluNAc compounds or derivatives or analogues).

The term “selectively and orthogonally protected hexosamine salts” refers to the salts of hexosamine which undergoes specific deprotection of one protective group in a multiply-protected hexosamine without affecting the other protected group. These selectively and orthogonally protected hexosamine salts are prepared by methods known in the literature and are used for the preparation of the compounds of the present invention. Preferred protected hexosamine salts include but not limited to hexosamine oxalates, hydrochlorides, hydrobromides, trifluoroacetate, tosylate, mesylate, and other sulfonate salts.

The term “at least one carboxylic acid” refers to an organic acid comprising a carboxyl group attached to an organic compound, wherein the organic compound is alkyl, alkenyl, alkynyl, aryl, cycloalkyl, heterocyclyl, heteroaryl which may be optionally substituted. For the purpose of the present invention, at least one carboxylic acid refers to a group comprising carboxylic acid with cycloalkyl or heterocyclyl. Cycloalkyl and heterocyclyl of the carboxylic acid may be bridged or unbridged, saturated or partially saturated or unsaturated and may be optionally substituted. The term at least one carboxylic acid includes but not limited to cycloalkylcarboxylic acid, cycloalkylacetic acid, and the like.

The term “coupling agent” refers to a chemical reagent capable of coupling two different compounds. For example the coupling reagent aids in coupling of acid with amine or alcohol to form an amide or ester. For the purpose of the present invention, the coupling agent includes but not limited to ethyl-3-(3-dimethylaminopropyl)carbodiimide hydrochloride, dicyclohexyl carbodiimide, diisopropylcarbodiimide, hexafluorophosphate azabenzotriazoletetramethyluronium, and the like.

The term “glycomimetic structure” refers to compounds with structures similar to carbohydrates/sugars but are modified to impart specific biological/physiological properties. In the present invention, compounds of Formula I are glycomimetic which are capable of modifying/inhibiting cell-cell interactions, cell-pathogen interactions or cell-extracellular matrix interactions, either directly or upon processing through the complex carbohydrate metabolic biosynthetic pathways.

The term “glycan” refers to oligosaccharide and polysaccharide compounds which have glycosidic linkages. Glycans also refers to carbohydrate/sugar part of the glycoproteins, glycolipids, proteoglycans or glyco-RNA.

The term “sialoglycans” refers to compounds comprising sialic acid and glycans.

The term “sialoglycoconjugates” are glycoconjugates of a glycoprotein containing sialic acid, glycolipid containing sialic acid (gangliosides), and sialic acid containing glycans attached to a ribonucleic acid (RNA) moiety.

The term “lectins” refers to group of proteins that are capable of binding with carbohydrates. The term “sialo-lectins” refers to compounds comprising lectins with sialic acid. The term “siglecs” refer to sialic acid-binding immunoglobulin-type lectins.

The term “epitopes” refers to an antigenic determinant which are part of an antigen and are recognized by the immune cells of the body. The epitopes are specific regions of proteins that can initiate an immune-cellular response mediated by T or B cells. Examples of epitopes include but not limited to sialic acid epitope, sialyl-Lewis-A epitope, sialyl-Lewis-X epitope, sulfo-sialyl-Lewis-X epitope, Lewis-X(CD15)/Y/A/B epitope, Sialyl-TF (Thomson-Friedenreich) antigen, and polysialicacid epitopes.

The term ‘MTOG’ refers to ‘mucin type O-glycosylation’, a class of glycosylation found on Ser/Thr present on extracellular domains of glycoproteins on the cell membrane as well as secreted glycoproteins which is characterized by the presence of α-GalNAc attached to Ser/Thr as the first and initiating monosaccharide.

The term ‘β-O-GlcNAc-ylation’ refers to a modification found commonly in nuclear and cytoplasmic proteins and is characterized by the lack of elaboration to oligosaccharide structures. β-O-GlcNAc-ylation of transcription factors are known to fine tune the cellular signaling processes, either in competitions with or in collaboration with other post-translational modifications (PTM) such as phosphorylation, acetylation, etc.

A term once described, the same meaning applies for it, throughout the disclosure.

As discussed in the background, there is a dire need of small molecules that are capable of modulating the glycan interactions including cell-cell interactions, cell-pathogen interactions, or cell-extracellular matrices interactions. These small molecules are pharmacologically effective compounds which can be further developed as drugs towards treatment of several diseases. Accordingly the present invention provides a compound of Formula-I of hexosamine compounds which are capable of modulating various interactions through the biosynthetic pathways. The present invention aims at providing a compound of Formula I which can exhibit varying degrees of hydrophobicity and hydrophilicity which assists in the modulation of glycan-glycan interactions in living systems through glycan biosynthesis and metabolism. The present invention provides a preparation method for the compounds of Formula I and methods of treatment or modification or prevention of a disease or a condition associated with interactions determined by the glycan biosynthetic pathways.

In an embodiment of the present invention, there is provided a compound of Formula I,

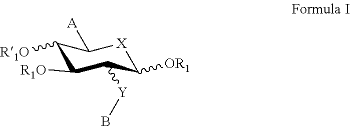

wherein A is C₁₋₁₂ alkyl, or —C₁₋₁₂ alkyl OR₁; wherein R₁ and R′₁ is independently selected from hydrogen, —C(O)C₁₋₁₂ alkyl, —C(O)C₂₋₁₂ alkenyl, or —C(O)C₂₋₁₂ alkynyl;
X is selected from O, S, Se, —CH₂, or —C(OH)R₂;
Y is —NR₂, or —CHR₂;
B is selected from hydrogen, —C(═Z)—(CH₂)ₘ—C₃₋₁₂ cycloalkyl, or —C(═Z)—(CH₂)ₘ—CH₂ heterocyclyl;
Z is O, or CH₂;
R₂ is selected from hydrogen, C₁₋₁₂ alkyl, C₂₋₁₂ alkenyl, C₂₋₁₂ alkynyl, or C₆₋₁₄aryl;
m is 0 to 4;
provided when —(OR′₁) is equatorial, —(Y—B) is either axial or equatorial,
and m=0, then B≠C(═Z)—(CH₂)ₘ—C₃ cycloalkyl

In an embodiment of the present invention, there is provided a compound of Formula I as disclosed herein, wherein R₁ and R′₁ is independently selected from hydrogen, or —C(O)C₁₋₃ alkyl;
X is O, or —CH₂;
Y is —NR₂;
B is hydrogen, or —C(═Z)—(CH₂)ₘ—C₃₋₁₂ cycloalkyl;
Z is O, or CH₂;
R₂ is hydrogen, or C₁₋₈ alkyl;
m is 0 or 1;
n is 0 to 7;
   provided when —(OR′₁) is equatorial, —(Y—B) is either axial or equatorial, and m=0, then B≠—C(═Z)—(CH₂)ₘ—C₃ cycloalkyl.

In an embodiment of the present invention, there is provided a compound of Formula I as disclosed herein, wherein R₁ and R′₁ is independently —C(O)C₁ alkyl; X is O; Y is —NH; B is —C(═Z)—(CH₂)ₘ—C₃₋₁₂ cycloalkyl; Z is O; m is 0 or 1; n is 0 to 7; provided when —(OR′₁) is equatorial, —(Y—B) is either axial or equatorial, and m=0, then B≠—C(═O)—C₃ cycloalkyl.

In an embodiment of the present invention, there is provided a compound of Formula I,

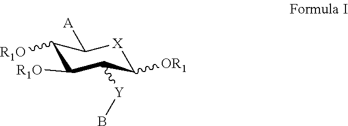

wherein A is C₁₋₁₂alkyl or —C₁₋₁₂ alkyl OR₁; wherein R₁ is selected from hydrogen, —C(O)C₁₋₁₂ alkyl, —C(O)C₂₋₁₂ alkenyl, or —C(O)C₂₋₁₂alkynyl; X is selected from O, S, Se, —CH₂, —C(OH)R₂, —CR₂R₃, NR₂, N⁺R₃R₄, —SR₂, or —P(O)OR₂; Y is —NR₂, or —CHR₂; B is selected from hydrogen, —C(═Z)—(CH₂)ₘ—C₃₋₁₂ unbridged cycloalkyl, —C(═Z)—(CH₂)ₘ—C₄₋₁₂ bridged cycloalkyl, —C(═Z)—(CH₂)ₘ—C₁₋₁₂ heterocyclyl, or —C(═Z)— (CH₂)ₘ—C₂₋₂₄ alkyl heterocyclyl; wherein —C(═Z)—(CH₂)ₘ—C₃-12 unbridged cycloalkyl, —C(═Z)—(CH₂)ₘ—C₄₋₁₂ bridged cycloalkyl, —C(═Z)—(CH₂)ₘ—C₁₋₁₂ heterocyclyl, or —C(═Z)— (CH₂)ₘ—C₂₋₂₄ alkyl heterocyclyl are optionally substituted with one or more substituents selected from ═O, C₁₋₁₂ alkyl, or C₁₋₁₂ alkyl-C(O)—C₁-₁₂ alkyl; Z is selected from O, CH₂, NR₃, or N⁺R₃R₄; R₂ is selected from hydrogen, C₁₋₁₂ alkyl, C₂₋₁₂ alkenyl, C₂₋₁₂ alkynyl, C₆₋₁₄aryl, C₇₋₂₆ arylalkyl, C₇₋₂₆ alkylaryl, N⁺R₅R₆R₇, —C(═Z)—(CH₂)ₘ—C₃-12 unbridged cycloalkyl, —C(═Z)—(CH₂)ₘ—C₄₋₁₂ bridged cycloalkyl, —C(═Z)—(CH₂)ₘ—C₁₋₁₂ heterocyclyl, or —C(═Z)— (CH₂)ₘ—C₂₋₂₄ alkyl heterocyclyl; wherein —C(═Z)—(CH₂)ₘ—C₃-12 unbridged cycloalkyl, —C(═Z)—(CH₂)ₘ—C₄₋₁₂ bridged cycloalkyl, —C(═Z)—(CH₂)ₘ—C₁₋₁₂ heterocyclyl, or —C(═Z)—(CH₂)ₘ—C₂₋₂₄ alkyl heterocyclyl are optionally substituted with one or more substituents selected from ═O, C₁₋₁₂ alkyl, or C₁₋₁₂ alkyl-C(O)—C₁₋₁₂ alkyl; R₃, and R₄ are independently selected from hydrogen, C₁₋₁₂ alkyl, C₂₋₁₂ alkenyl, C₂-12alkynyl, C₆₋₁₄aryl, C₇₋₂₆arylalkyl, C₇₋₂₆alkylaryl, or N⁺R₅R₆R₇; R₅, R₆, and R₇ are independently selected from hydrogen, C₁₋₁₂ alkyl, C₂₋₁₂ alkenyl, C₂₋₁₂ alkynyl, or C₆₋₁₄aryl; and m is 0 to 7.

In an embodiment of the present invention, there is provided a compound of Formula I, wherein A is C₁₋₁₀alkyl or C₁₋₁₀alkyl —OR₁; R₁ is selected from hydrogen, —C(O)C₁₋₁₀alkyl, —C(O)C₂₋₁₀alkenyl, or —C(O)C₂₋₁₀alkynyl; X is selected from O, S, Se, —CH₂, —C(OH)R₂, —CR₂R₃, NR₂, N⁺R₃R₄, —SR₂, or —P(O)OR₂; Y is —NR₂, or —CHR₂; B is selected from hydrogen, —C(═Z)—(CH₂)ₘ—C₃-12 unbridged cycloalkyl, —C(═Z)—(CH₂)ₘ—C₄₋₁₂ bridged cycloalkyl, —C(═Z)—(CH₂)ₘ—C₁₋₁₂ heterocyclyl, or —C(═Z)— (CH₂)ₘ—C₂₋₂₄ alkyl heterocyclyl; wherein —C(═Z)—(CH₂)ₘ C₃₋₁₂ unbridged cycloalkyl, —C(═Z)—(CH₂)ₘ—C₄₋₁₂ bridged cycloalkyl, —C(═Z)—(CH₂)ₘ—C₁₋₁₂heterocyclyl, or —C(═Z)— (CH₂)ₘ—C₂₋₂₄ alkyl heterocyclyl are optionally substituted with one or more substituents selected from ═O, C₁₋₁₀ alkyl, or C₁₋₁₀alkyl-C(O)—C₁₋₁₀ alkyl; Z is selected from O, CH₂, NR₃, or N⁺R₃R₄; R₂ is selected from hydrogen, C₁₋₁₀ alkyl, C₂₋₁₀ alkenyl, C₂₋₁₀ alkynyl, C₆₋₁₂aryl, C₇₋₂₂arylalkyl, C₇-22alkylaryl, N⁺R₅R₆R₇, —C(═Z)—(CH₂)ₘ—C₃₋₁₂ unbridged cycloalkyl, —C(═Z)—(CH₂)ₘ C₄₋₁₂ bridged cycloalkyl, —C(═Z)—(CH₂)ₘC₁₋₁₂ heterocyclyl, or —C(═Z)— (CH₂)ₘ—C₂-24 alkyl heterocyclyl; wherein —C(═Z)—(CH₂)ₘ—C₃-12 unbridged cycloalkyl, —C(═Z)—(CH₂)ₘ—C₄₋₁₂ bridged cycloalkyl, —C(═Z)—(CH₂)ₘ—C₁₋₁₂ heterocyclyl, or —C(═Z)—(CH₂)ₘ—C₂₋₂₄ alkyl heterocyclyl are optionally substituted with one or more substituents selected from ═O, C₁₋₁₀ alkyl, or C₁₋₁₀ alkyl-C(O)—C₁₋₁₀ alkyl; R₃, and R₄ are independently selected from hydrogen, C₁₋₁₀ alkyl, C₂₋₁₀ alkenyl, C₂₋₁₀ alkynyl, C₆₋₁₂aryl, C₇₋₂₂arylalkyl, C₇₋₂₂alkylaryl, or N⁺R₅R₆R₇; wherein R₅, R₆, and R₇ are independently selected from hydrogen, C₁₋₁₀ alkyl, C₂₋₁₀ alkenyl, C₂₋₁₀ alkynyl, or C₆₋₁₂aryl; and m is 0 to 7.

In an embodiment of the present invention, there is provided a compound of Formula I, wherein the compound is selected from

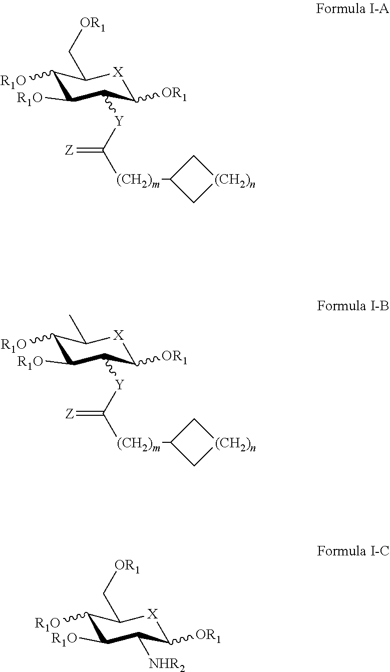

wherein R₁ is selected from hydrogen, —C(O)C₁₋₁₂ alkyl, —C(O)C₂₋₁₂ alkenyl, or —C(O)C₂₋₁₂alkynyl; X is selected from O, S, Se, —CH₂, —C(OH)R₂, —CR₂R₃, NR₂, N⁺R₃R₄, —SR₂, or —P(O)OR₂; Y is —NR₂, or —CHR₂; Z is selected from O, CH₂, NR₃, or N⁺R₃R₄; R₂ is selected from hydrogen, C₁₋₁₂ alkyl, C₂₋₁₂ alkenyl, C₂₋₁₂ alkynyl, C₆₋₁₄aryl, C₇₋₂₆ arylalkyl, C₇₋₂₆ alkylaryl, N⁺R₅R₆R₇, —C(═Z)—(CH₂)ₘ—C₃-12 unbridged cycloalkyl, —C(═Z)—(CH₂)ₘ—C₄₋₁₂ bridged cycloalkyl, —C(═Z)—(CH₂)ₘ—C₁₋₁₂ heterocyclyl, or —C(═Z)— (CH₂)ₘ—C₂₋₂₄ alkyl heterocyclyl; wherein —C(═Z)—(CH₂)ₘ C₃-12 unbridged cycloalkyl, —C(═Z)—(CH₂)ₘ—C₄₋₁₂ bridged cycloalkyl, —C(═Z)—(CH₂)ₘ—C₁₋₁₂ heterocyclyl, or —C(═Z)— (CH₂)ₘ—C₂₋₂₄ alkyl heterocyclyl are optionally substituted with one or more substituents selected from ═O, C₁₋₁₂ alkyl, or C₁₋₁₂ alkyl-C(O)—C₁₋₁₂ alkyl; R₃, and R₄ are independently selected from hydrogen, C₁₋₁₂ alkyl, C₂₋₁₂ alkenyl, C₂₋₁₂ alkynyl, C₆₋₁₄aryl, C₇₋₂₆ arylalkyl, C₇₋₂₆ alkylaryl, or N⁺R₅R₆R₇; R₅, R₆, and R₇ are independently selected from hydrogen, C₁₋₁₂ alkyl, C₂₋₁₂ alkenyl, C₂₋₁₂ alkynyl, or C₆₋₁₄aryl; m is 0 to 7; and n is 0 to 10.

In an embodiment of the present invention, there is provided a compound of Formula I, wherein the compound is selected from Formula I-A, Formula I-B, or Formula I-C, wherein R₁ is hydrogen, or —C(O)C₁₋₁₂ alkyl; X is O, or —CH₂; Y is —NR₂, or —CHR₂; Z is O, or CH₂; R₂ is selected from hydrogen, C₁₋₁₂ alkyl, C₂₋₁₂ alkenyl, C₂₋₁₂ alkynyl, C₆₋₁₄aryl, C₇₋₂₆ arylalkyl, C₇₋₂₆ alkylaryl, N⁺R₅R₆R₇, —C(═Z)—(CH₂)ₘ—C₃-12 unbridged cycloalkyl, or —C(═Z)—(CH₂)ₘ—C₄₋₁₂ bridged cycloalkyl; wherein —C(═Z)—(CH₂)ₘ—C₃-12 unbridged cycloalkyl, or —C(═Z)—(CH₂)ₘ—C₄₋₁₂ bridged cycloalkyl, are optionally substituted with one or more substituents selected from ═O, or C₁₋₁₂ alkyl; R₅, R₆, and R₇ are independently selected from hydrogen, C₁₋₁₂ alkyl, C₂₋₁₂ alkenyl, C₂₋₁₂ alkynyl, or C₆₋₁₄aryl; m is 0 or 1; and n is 0 to 7.

In an embodiment of the present invention, there is provided a compound of Formula I, wherein R₁ is selected from hydrogen, —C(O)C₁₋₈ alkyl, —C(O)C₂₋₈ alkenyl, or —C(O)C₂₋₈alkynyl; X is selected from O, S, Se, —CH₂, —C(OH)R₂, —CR₂R₃, NR₂, N⁺R₃R₄, —SR₂, or —P(O)OR₂; Y is —NR₂, or —CHR₂; Z is selected from O, CH₂, NR₃, or N⁺R₃R₄; R₂ is selected from hydrogen, C₁₋₈ alkyl, C₂₋₈ alkenyl, C₂₋₈ alkynyl, C₆₋₁₂aryl, C₇₋₂₀arylalkyl, C₇₋₂₀alkylaryl, N⁺R₅R₆R₇, —C(═Z)—(CH₂)ₘ—C₃₋₁₂ unbridged cycloalkyl, —C(═Z)—(CH₂)ₘ—C₄₋₁₂ bridged cycloalkyl, —C(═Z)—(CH₂)ₘ—C₁₋₁₂ heterocyclyl, or —C(═Z)— (CH₂)ₘ—C₂₋₂₄ alkyl heterocyclyl; wherein —C(═Z)—(CH₂)ₘ—C₃-12 unbridged cycloalkyl, —C(═Z)—(CH₂)ₘ—C₄₋₁₂ bridged cycloalkyl, —C(═Z)—(CH₂)ₘ—C₁₋₁₂ heterocyclyl, or —C(═Z)— (CH₂)ₘ—C₂₋₂₄ alkyl heterocyclyl are optionally substituted with one or more substituents selected from ═O, C₁₋₈ alkyl, or C₁₋₈ alkyl-C(O)—C₁₋₈ alkyl; R₃, and R₄ are independently selected from hydrogen, C₁₋₈ alkyl, C₂₋₈ alkenyl, C₂₋₈ alkynyl, C₆₋₁₂aryl, C₇₋₂₀arylalkyl, C₇₋₂₀alkylaryl, or N⁺R₅R₆R₇; R₅, R₆, and R₇ are independently selected from hydrogen, C₁₋₈ alkyl, C₂₋₈ alkenyl, C₂₋₈ alkynyl, or C₆₋₁₂aryl; m is 0 or 1; and n is 0 to 7.

In an embodiment of the present invention, there is provided a compound selected from

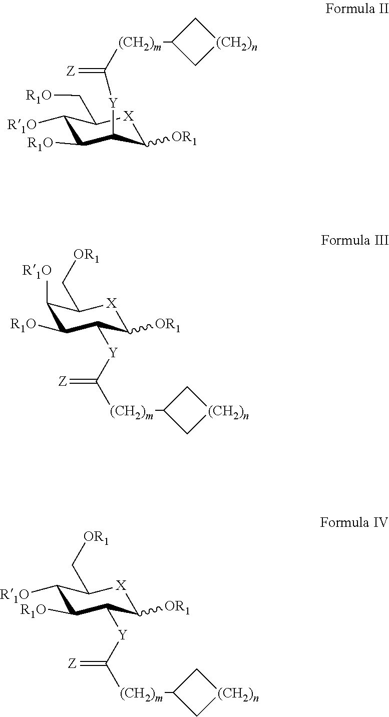

wherein R₁ and R′₁ is independently selected from hydrogen, or —C(O)C₁₋₃ alkyl; X is O; Y is —NR₂; Z is O; R₂ is hydrogen, or C₁₋₈ alkyl; m is 0 or 1,
n is 0 to 7;
when m of Formula II is 0, then n is 1 to 7;
when m of Formula IV is 0, then n is 1 to 7;
when m of Formula II is 1, then n is 0 to 7; and when m of Formula IV is 1, then n is 0 to 7.

In an embodiment of the present invention, there is provided a compound selected from

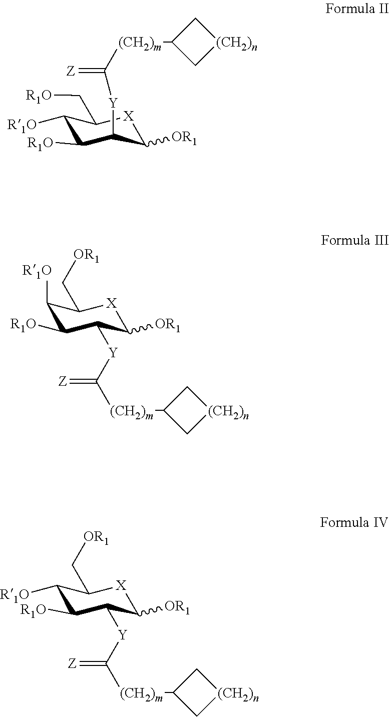

wherein R₁ and R′₁ is independently —C(O)C₁ alkyl; X is O; Y is —NH; Z is O; for Formula III when m is 0 or 1, then n is 0 to 7;
when m of Formula II is 0, then n is 1 to 7;
when m of Formula IV is 0, then n is 1 to 7;
when m of Formula II is 1, then n is 0 to 7; and when m of Formula IV is 1, then n is 0 to 7.

In an embodiment of the present invention, there is provided a compound of Formula II, wherein R₁ and R′₁ is independently selected from hydrogen, or —C(O)C₁₋₃ alkyl; X is O; Y is —NR₂; Z is O; R₂ is hydrogen, or C₁₋₈ alkyl; when m of Formula II is 0, then n is 1 to 7 and when m of Formula II is 1, then n is 0 to 7.

In an embodiment of the present invention, there is provided a compound of Formula III, wherein R₁ and R′₁ is independently selected from hydrogen, or —C(O)C₁₋₃ alkyl; X is O; Y is —NR₂; Z is O; R₂ is hydrogen, or C₁₋₈ alkyl; m is 0 or 1; and n is 0 to 7.

In an embodiment of the present invention, there is provided a compound of Formula IV, wherein R₁ and R′₁ is independently selected from hydrogen, or —C(O)C₁₋₃ alkyl; X is O; Y is —NR₂; Z is O; R₂ is hydrogen, or C₁₋₈ alkyl; when m of Formula IV is 0, then n is 1 to 7; and when m of Formula IV is 1, then n is 0 to 7.

In an embodiment of the present invention, there is provided a compound of Formula I, wherein the compound is selected from

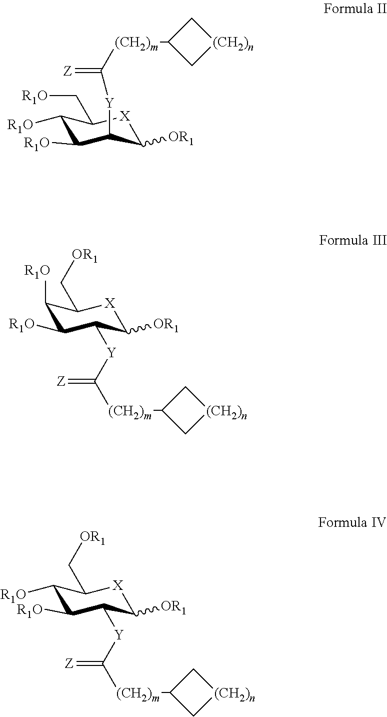

wherein R₁ is selected from hydrogen, —C(O)C₁₋₁₂ alkyl, —C(O)C₂₋₁₂ alkenyl, or —C(O)C₂₋₁₂ alkynyl; X is selected from O, S, Se, —CH₂, —C(OH)R₂, —CR₂R₃, NR₂, N⁺R₃R₄, —SR₂, or —P(O)OR₂; Y is —NR₂, or —CHR₂; Z is selected from O, CH₂, NR₃, or N⁺R₃R₄; R₂ is selected from hydrogen, C₁₋₁₂ alkyl, C₂₋₁₂ alkenyl, C₂₋₁₂ alkynyl, C₆₋₁₄aryl, C₇₋₂₆ arylalkyl, C₇₋₂₆ alkylaryl, N⁺R₅R₆R₇, —C(═Z)—(CH₂)ₘ—C₃₋₁₂ unbridged cycloalkyl, —C(═Z)—(CH₂)ₘ—C₄₋₁₂ bridged cycloalkyl, —C(═Z)—(CH₂)ₘ—C₁₋₁₂ heterocyclyl, or —C(═Z)— (CH₂)ₘ—C₂₋₂₄ alkyl heterocyclyl; wherein —C(═Z)—(CH₂)ₘ C₃₋₁₂ unbridged cycloalkyl, —C(═Z)—(CH₂)ₘ—C₄₋₁₂ bridged cycloalkyl, —C(═Z)—(CH₂)ₘ—C₁₋₁₂ heterocyclyl, or —C(═Z)— (CH₂)ₘ—C₂₋₂₄ alkyl heterocyclyl are optionally substituted with one or more substituents selected from ═O, C₁₋₁₂ alkyl, or C₁₋₁₂ alkyl-C(O)—C₁₋₁₂ alkyl; R₃, and R₄ are independently selected from hydrogen, C₁₋₁₂ alkyl, C₂₋₁₂ alkenyl, C₂₋₁₂ alkynyl, C₆₋₁₄aryl, C₇₋₂₆ arylalkyl, C₇₋₂₆ alkylaryl, or N⁺R₅R₆R₇; R₅, R₆, and R₇ are independently selected from hydrogen, C₁₋₁₂ alkyl, C₂₋₁₂ alkenyl, C₂₋₁₂ alkynyl, or C₆₋₁₄aryl; m is 0 to 7; and n is 0 to 10.

In an embodiment of the present invention, there is provided a compound of Formula I, wherein R₂ is selected from

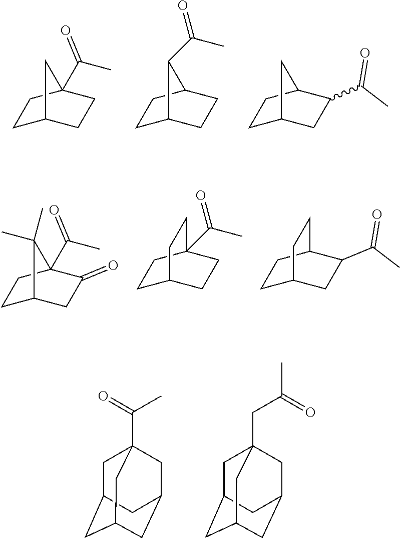

In an embodiment of the present invention, there is provided a compound of Formula I, wherein the compound is selected from
i. (3S,4R,5S,6R)-6-(acetoxymethyl)-3-(cyclobutanecarboxamido)tetrahydro-2H-pyran-2,4,5-triyl triacetate/Acetyl 3,4,6-tri-O-acetyl-2-cyclobutanecarboxamido-2-deoxy-α/β-D-manno-hexopyranoside
ii. (3S,4R,5S,6R)-6-(acetoxymethyl)-3-(cyclopentanecarboxamido)tetrahydro-2H-pyran-2,4,5-triyl triacetate/Acetyl 3,4,6-tri-O-acetyl-2-cyclopentanecarboxamido-2-deoxy-α/β-D-manno-hexopyranoside
iii. (3S,4R,5S,6R)-6-(acetoxymethyl)-3-(cyclohexanecarboxamido)tetrahydro-2H-pyran-2,4,5-triyl triacetate/Acetyl 3,4,6-tri-O-acetyl-2-cyclohexanecarboxamido-2-deoxy-α/β-D-manno-hexopyranoside
iv. (3S,4R,5S,6R)-6-(acetoxymethyl)-3-(cycloheptanecarboxamido)tetrahydro-2H-pyran-2,4,5-triyl triacetate/Acetyl 3,4,6-tri-O-acetyl-2-cycloheptanecarboxamido-2-deoxy-α/β-D-manno-hexopyranoside
v. (3R,4R,5R,6R)-6-(acetoxymethyl)-3-(cyclopropanecarboxamido)tetrahydro-2H-pyran-2,4,5-triyl triacetate/Acetyl 3,4,6-tri-O-acetyl-2-cyclopropanecarboxamido-2-deoxy-α/β-D-galacto-hexopyranoside
vi. (3R,4R,5R,6R)-6-(acetoxymethyl)-3-(cyclobutanecarboxamido)tetrahydro-2H-pyran-2,4,5-triyl triacetate/Acetyl 3,4,6-tri-O-acetyl-2-cyclobutanecarboxamido-2-deoxy-α/β-D-galacto-hexopyranoside
vii. (3R,4R,5R,6R)-6-(acetoxymethyl)-3-(cyclopentanecarboxamido)tetrahydro-2H-pyran-2,4,5-triyl triacetate/Acetyl 3,4,6-tri-O-acetyl-2-cyclopentanecarboxamido-2-deoxy-α/β-D-galacto-hexopyranoside
viii. (3R,4R,5R,6R)-6-(acetoxymethyl)-3-(cyclohexanecarboxamido)tetrahydro-2H-pyran-2,4,5-triyl triacetate/Acetyl 3,4,6-tri-O-acetyl-2-cyclohexanecarboxamido-2-deoxy-α/β-D-galacto-hexopyranoside
ix. (3R,4R,5R,6R)-6-(acetoxymethyl)-3-(cycloheptanecarboxamido)tetrahydro-2H-pyran-2,4,5-triyl triacetate/Acetyl 3,4,6-tri-O-acetyl-2-cycloheptanecarboxamido-2-deoxy-α/β-D-galacto-hexopyranoside
x. (3R,4R,5S,6R)-6-(acetoxymethyl)-3-(cyclobutanecarboxamido)tetrahydro-2H-pyran-2,4,5-triyl triacetate/Acetyl 3,4,6-tri-O-acetyl-2-cyclobutanecarboxamido-2-deoxy-α/β-D-gluco-hexopyranoside
xi. (3R,4R,5S,6R)-6-(acetoxymethyl)-3-(cyclopentanecarboxamido)tetrahydro-2H-pyran-2,4,5-triyl triacetate/Acetyl 3,4,6-tri-O-acetyl-2-cyclopentanecarboxamido-2-deoxy-α/β-D-gluco-hexopyranoside
xii. (3R,4R,5S,6R)-6-(acetoxymethyl)-3-(cyclohexanecarboxamido)tetrahydro-2H-pyran-2,4,5-triyl triacetate/Acetyl 3,4,6-tri-O-acetyl-2-cyclohexanecarboxamido-2-deoxy-α/β-D-gluco-hexopyranoside
xiii. (3R,4R,5S,6R)-6-(acetoxymethyl)-3-(cycloheptanecarboxamido)tetrahydro-2H-pyran-2,4,5-triyl triacetate/Acetyl 3,4,6-tri-O-acetyl-2-cycloheptanecarboxamido-2-deoxy-α/β-D-gluco-hexopyranoside
xiv. (3S,4R,5S,6R)-6-(acetoxymethyl)-3-(2-cyclopropylacetamido)tetrahydro-2H-pyran-2,4,5-triyl triacetate/Acetyl 3,4,6-tri-O-acetyl-2-cyclopropylacetamido-2-deoxy-α/β-D-manno-hexopyranoside
xv. (3S,4R,5S,6R)-6-(acetoxymethyl)-3-(2-cyclobutylacetamido)tetrahydro-2H-pyran-2,4,5-triyl triacetate/Acetyl 3,4,6-tri-O-acetyl-2-cyclobutylacetamido-2-deoxy-α/β-D-manno-hexopyranoside
xvi. (3S,4R,5S,6R)-6-(acetoxymethyl)-3-(2-cyclopentylacetamido)tetrahydro-2H-pyran-2,4,5-triyl triacetate/Acetyl 3,4,6-tri-O-acetyl-2-cyclopentylacetamido-2-deoxy-α/β-D-manno-hexopyranoside
xvii. (3S,4R,5S,6R)-6-(acetoxymethyl)-3-(2-cyclohexylacetamido)tetrahydro-2H-pyran-2,4,5-triyl triacetate/Acetyl 3,4,6-tri-O-acetyl-2-cyclohexylacetamido-2-deoxy-α/β-D-manno-hexopyranoside
xviii. (3S,4R,5S,6R)-6-(acetoxymethyl)-3-(2-cycloheptylacetamido)tetrahydro-2H-pyran-2,4,5-triyl triacetate/Acetyl 3,4,6-tri-O-acetyl-2-cycloheptylacetamido-2-deoxy-α/β-D-manno-hexopyranoside
xix. (3R,4R,5R,6R)-6-(acetoxymethyl)-3-(2-cyclopropylacetamido)tetrahydro-2H-pyran-2,4,5-triyl triacetate/Acetyl 3,4,6-tri-O-acetyl-2-cyclopropylacetamido-2-deoxy-α/β-D-galacto-hexopyranoside
xx. (3R,4R,5R,6R)-6-(acetoxymethyl)-3-(2-cyclobutylacetamido)tetrahydro-2H-pyran-2,4,5-triyl triacetate/Acetyl 3,4,6-tri-O-acetyl-2-cyclobutylacetamido-2-deoxy-α/β-D-galacto-hexopyranoside
xxi. (3R,4R,5R,6R)-6-(acetoxymethyl)-3-(2-cyclopentylacetamido)tetrahydro-2H-pyran-2,4,5-triyl triacetate/Acetyl 3,4,6-tri-O-acetyl-2-cyclopentylacetamido-2-deoxy-α/β-D-galacto-hexopyranoside
xxii. (3R,4R,5R,6R)-6-(acetoxymethyl)-3-(2-cyclohexylacetamido)tetrahydro-2H-pyran-2,4,5-triyl triacetate/Acetyl 3,4,6-tri-O-acetyl-2-cyclohexylacetamido-2-deoxy-α/β-D-galacto-hexopyranoside
xxiii. (3R,4R,5R,6R)-6-(acetoxymethyl)-3-(2-cycloheptylacetamido)tetrahydro-2H-pyran-2,4,5-triyl triacetate/Acetyl 3,4,6-tri-O-acetyl-2-cycloheptylacetamido-2-deoxy-α/β-D-galacto-hexopyranoside
xxiv. (3R,4R,5S,6R)-6-(acetoxymethyl)-3-(2-cyclopropylacetamido)tetrahydro-2H-pyran-2,4,5-triyl triacetate/Acetyl 3,4,6-tri-O-acetyl-2-cyclopropylacetamido-2-deoxy-α/β-D-gluco-hexopyranoside
xxv. (3R,4R,5S,6R)-6-(acetoxymethyl)-3-(2-cyclobutylacetamido)tetrahydro-2H-pyran-2,4,5-triyl triacetate/Acetyl 3,4,6-tri-O-acetyl-2-cyclobutylacetamido-2-deoxy-α/β-D-gluco-hexopyranoside
xxvi. (3R,4R,5S,6R)-6-(acetoxymethyl)-3-(2-cyclopentylacetamido)tetrahydro-2H-pyran-2,4,5-triyl triacetate/Acetyl 3,4,6-tri-O-acetyl-2-cyclopentylacetamido-2-deoxy-α/β-D-gluco-hexopyranoside
xxvii. (3R,4R,5S,6R)-6-(acetoxymethyl)-3-(2-cyclohexylacetamido)tetrahydro-2H-pyran-2,4,5-triyl triacetate/Acetyl 3,4,6-tri-O-acetyl-2-cyclohexylacetamido-2-deoxy-α/β-D-gluco-hexopyranoside
xxviii. (3R,4R,5S,6R)-6-(acetoxymethyl)-3-(2-cycloheptylacetamido)tetrahydro-2H-pyran-2,4,5-triyl triacetate/Acetyl 3,4,6-tri-O-acetyl-2-cycloheptylacetamido-2-deoxy-α/β-D-gluco-hexopyranoside.

In an embodiment of the present invention, there is provided a process for preparing the compound of Formula I, the process comprising reacting at least one selectively and orthogonally protected hexosamine salt with at least one carboxylic acid to obtain the compound of Formula I.

In an embodiment of the present invention, there is provided a process for preparing the compound of Formula I, the process comprising reacting at least one selectively and orthogonally protected hexosamine salt of Formula A, with at least one carboxylic acid of Formula R₂CO₂H to obtain the compound of Formula I,

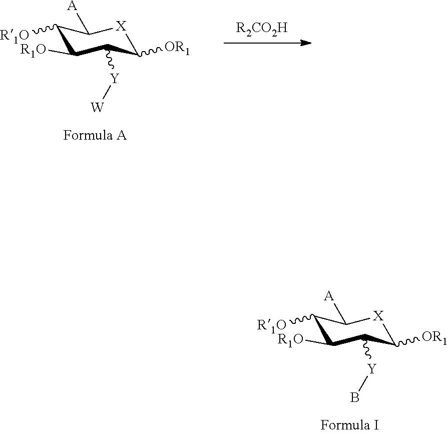

wherein R₂ is selected from C₃₋₁₂ cycloalkyl-(CH₂)ₘ—, C₁₋₁₂ heterocyclyl-(CH₂)ₘ, or C₂₋₂₄ alkyl heterocyclyl)-(CH₂)ₘ—;
W is selected from hydrogen, or C₁₋₁₂ alkyl;
A is C₁₋₁₂ alkyl, or —C₁₋₁₂ alkyl OR₁;
R₁ and R′₁ is independently selected from hydrogen —C(O)C₂₋₁₂ alkenyl, or —C(O)C₂₋₁₂ alkynyl;
X is selected from O, S, Se, —CH₂, or —C(OH)R₂,
Y is —NR₂, or —CHR₂;
B is selected from hydrogen, —C(═Z)—(CH₂)ₘ—C₃₋₁₂ cycloalkyl, or —C(═Z)—(CH₂)ₘ—C₁₋₁₂ heterocyclyl;
Z is O, or CH₂;
R₂ is selected from hydrogen, C₁₋₁₂ alkyl, C₂₋₁₂ alkenyl, C₂₋₁₂ alkynyl, or C₆₋₁₄aryl;
m is 0 to 7;
provided when —(OR′₁) is equatorial, —(Y—B) is either axial or equatorial,
and m=0, then B≠C(═Z)—(CH₂)ₘ—C₃ cycloalkyl

In an embodiment of the present invention, there is provided a process for preparing the compound of Formula I, wherein the process is carried out in the presence of a coupling agent, a base, a solvent, or combinations thereof at a temperature in the range of 0° C. to 100° C. In another embodiment of the present invention, there is provided a process for preparing the compound of Formula I, wherein the process is carried out in the presence of a coupling agent, a base, a solvent, or combinations thereof at a temperature in the range of 10° C. to 80° C. In yet another embodiment of the present invention, there is provided a process for preparing the compound of Formula I, wherein the process is carried out in the presence of a coupling agent, a base, a solvent, or combinations thereof at a temperature in the range of 20° C. to 60° C.

In an embodiment of the present invention, there is provided a process for preparing the compound of Formula I, wherein the coupling agent is selected from 1-ethyl-3-(3-dimethylaminopropyl)carbodiimide hydrochloride, dicyclohexylcarbodiimide, diisopropylcarbodiimide, hexafluorophosphateazabenzotriazole tetramethyluronium, 1,1′-carbonyldiimidazole, 1-hydroxybenzytriaole, or combinations thereof; the base is selected from pyridine, triethylamine, 4-(N,N-dimethylamino)pyridine, sodium bi-carbonate, sodium carbonate, lithium carbonate, ammonium bicarbonate, or combinations thereof; and the solvent is selected from dimethyl formamide, dioxan, dichloromethane, chloroform, acetonitrile, ethyleneglycol, tetrahydrofuran, cyclohexane, or combinations thereof.

In an embodiment of the present invention, there is provided a process for preparing the compound of Formula I, the process comprising reacting at least one selectively and orthogonally protected hexosamine salt with at least one carboxylic acid in the presence of a coupling agent selected from 1-ethyl-3-(3-dimethylaminopropyl)carbodiimide hydrochloride, dicyclohexylcarbodiimide, diisopropylcarbodiimide, hexafluorophosphate azabenzotriazole tetramethyluronium, 1,1′-carbonyldiimidazole, 1-hydroxybenzytriaole, or combinations thereof a base selected from pyridine, triethylamine, 4-(N,N-dimethylamino)pyridine, sodium bi-carbonate, sodium carbonate, lithium carbonate, ammonium bicarbonate, or combinations thereof, a solvent selected from dimethyl formamide, dioxan, dichloromethane, chloroform, acetonitrile, ethyleneglycol, tetrahydrofuran, cyclohexane, or combinations thereof at a temperature in the range of 0° C. to 100° C. to obtain the compound of Formula I.

In an embodiment of the present invention, there is provided a process for preparing the compound of Formula I, the process comprising reacting at least one selectively and orthogonally protected hexosamine salt with at least one carboxylic acid in the presence of 1-ethyl-3-(3-dimethylaminopropyl)carbodiimide hydrochloride, a base selected from pyridine, triethylamine, 4-(N,N-dimethylamino)pyridine, or combinations thereof, a solvent selected from dimethyl formamide, dioxan, dichloromethane, chloroform, acetonitrile, ethyleneglycol, tetrahydrofuran, cyclohexane, or combinations thereof at a temperature in the range of 0° C. to 100° C. to obtain the compound of Formula I.

In an embodiment of the present invention, there is provided a compound of Formula I, wherein the compound of Formula I is a glycomimetic structure for modulating sialoglycans, mucin-type O-glycans (MTOG), selectins, siglecs, β-O-GlcNAc-ylation, or combinations thereof.

In an embodiment of the present invention, there is provided a compound selected from a compound of Formula II, Formula III or Formula IV, wherein the compound is a glycomimetic structure for modulating sialoglycans, mucin-type O-glycans (MTOG), selectins, siglecs, β-O-GlcNAc-ylation, or combinations thereof

In an embodiment of the present invention, there is provided a compound of Formula I, wherein the compound of Formula I is capable of resulting in a glycomimetic structure upon processing through the complex carbohydrate metabolism and glycan biosynthetic pathways, for modifying sialoglycans, mucin-type O-glycans (MTOG), selectins, siglecs, β-O-GlcNAc-ylation, or combinations thereof.

In an embodiment of the present invention, there is provided a compound selected from a compound of Formula II, Formula III or Formula IV, wherein the compound is capable of resulting in a glycomimetic structure upon processing through the complex carbohydrate metabolism and glycan biosynthetic pathways, for modifying sialoglycans, mucin-type O-glycans (MTOG), selectins, siglecs, β-O-GlcNAc-ylation, or combinations thereof.

In an embodiment of the present invention, there is provided a compound of Formula I, wherein the compound of Formula I modifies cell-cell interactions, cell-pathogen interactions, or cell-extracellular matrix interactions.

In an embodiment of the present invention, there is provided a compound selected from a compound of Formula II, Formula III or Formula IV, wherein the compound modifies cell-cell interactions, cell-pathogen interactions, or cell-extracellular matrix interactions

In an embodiment of the present invention, there is provided a compound of Formula I, wherein the compound of Formula I inhibits, or alters, or enhances, or modulates cell-cell interactions, cell-pathogen interactions, or cell-extracellular matrix interactions.

In an embodiment of the present invention, there is provided a compound selected from a compound of Formula II, Formula III or Formula IV, wherein the compound inhibits, or alters, or enhances, or modulates cell-cell interactions, cell-pathogen interactions, or cell-extracellular matrix interactions

In an embodiment of the present invention, there is provided a compound of Formula I, wherein the compound of Formula I is capable of modulating glycan-protein interactions, glycan-glycan interactions, sialic acids-proteins interactions, plant-pathogen interactions, sialyl-Lewis X/Lewis Y-selectins interactions, sialoglycoconjugates-siglecs interactions, polysialic acid-sialo-lectins interactions, sialic acid-neuraminidases interactions, sialic acid-hemagglutinin interactions, modulation of polysialic acid-protein interactions, sialic acid-microbial adhesin interactions, or combinations thereof.

In an embodiment of the present invention, there is provided a compound selected from a compound of Formula II, Formula III or Formula IV, wherein the compound is capable of modulating glycan-protein interactions, glycan-glycan interactions, sialic acids-proteins interactions, plant-pathogen interactions, sialyl-Lewis X/Lewis Y-selectins interactions, sialoglycoconjugates-siglecs interactions, polysialic acid-sialo-lectins interactions, sialic acid-neuraminidases interactions, sialic acid-hemagglutinin interactions, modulation of polysialic acid-protein interactions, sialic acid-microbial adhesin interactions, or combinations thereof

In an embodiment of the present invention, there is provided a compound of Formula I, wherein the compound is an adjunct in combinatorial cancer chemotherapy or an adjunct in combinatorial cancer immunotherapy.

In an embodiment of the present invention, there is provided a compound selected from a compound of Formula II, Formula III or Formula IV, wherein the compound is an adjunct in combinatorial cancer chemotherapy or an adjunct in combinatorial cancer immunotherapy

In an embodiment of the present invention, there is provided a compound of Formula I for use in method of prevention or treatment or modification of a condition or a disease, the method comprising administering the compound of Formula I as disclosed herein to a subject in need thereof.

In an embodiment of the present invention, there is provided a compound selected from a compound of Formula II, Formula III or Formula IV for use in method of prevention or treatment or modification of a condition or a disease, the method comprising administering the compound of Formula I as disclosed herein to a subject in need thereof.

In an embodiment of the present invention, there is provided a compound of Formula I, Formula II, Formula III or Formula IV, for use in method of prevention or treatment or modification of a condition or a disease selected from acute inflammatory disorders, chronic inflammatory disorders, asthma, allergy, psoriasis, rheumatoid arthritis, tumor metastasis, cancer metastasis, reperfusion syndrome, cytokine storm, viral infectious diseases, bacterial infectious diseases, acute inflammation of liver, neutrophil infiltration, xenograft maintenance, neoplasm, tuberculosis, acute liver injury, acute kidney injury, thrombosis, chronic obstructive pulmonary disease (COPD), hay fever, stroke, atherosclerosis, rhinitis, contact dermatitis, atopic dermatitis, inflammatory bowel disease, multiple sclerosis, type I diabetes, organ transplant rejection, systemic lupus erythematosus (SLE), cystic fibrosis, congenital disorders of glycosylation, leukocyte adhesion deficiency disorders, or fertilization disorder, the method comprising administering the compound of Formula I as disclosed herein to a subject in need thereof.

In an embodiment of the present invention, there is provided a pharmaceutical composition comprising the compound of Formula I as disclosed herein optionally with at least one pharmaceutically acceptable salt thereof.

In an embodiment of the present invention, there is provided a pharmaceutical composition comprising the compound of Formula II as disclosed herein optionally with at least one pharmaceutically acceptable salt thereof.

In an embodiment of the present invention, there is provided a pharmaceutical composition comprising the compound of Formula III as disclosed herein optionally with at least one pharmaceutically acceptable salt thereof.

In an embodiment of the present invention, there is provided a pharmaceutical composition comprising the compound of Formula IV as disclosed herein optionally with at least one pharmaceutically acceptable salt thereof.

In an embodiment of the present invention, there is provided a pharmaceutical composition comprising the compound of Formula I as disclosed herein optionally with at least one pharmaceutically acceptable salt thereof, wherein the pharmaceutical composition is capable of inhibiting or modulating or altering or enhancing sialoglycans, mucin-type O-glycans (MTOG), selectins, siglecs, j-O-GlcNAc-ylation, or combinations thereof.

In an embodiment of the present invention, there is provided a pharmaceutical composition comprising the compound selected from Formula II, Formula III, or Formula IV, as disclosed herein optionally with at least one pharmaceutically acceptable salt thereof, wherein the pharmaceutical composition is capable of inhibiting or modulating or altering or enhancing sialoglycans, mucin-type O-glycans (MTOG), selectins, siglecs, β-O-GlcNAc-ylation, or combinations thereof

In an embodiment of the present invention, there is provided a pharmaceutical composition comprising the compound of Formula I as disclosed herein optionally with at least one pharmaceutically acceptable salt thereof, wherein the pharmaceutical composition modifies cell-cell interactions, cell-pathogen interactions, or cell-extracellular matrix interactions.

In an embodiment of the present invention, there is provided a pharmaceutical composition comprising the compound selected from Formula II, Formula III, or Formula IV, as disclosed herein optionally with at least one pharmaceutically acceptable salt thereof, wherein the pharmaceutical composition modifies cell-cell interactions, cell-pathogen interactions, or cell-extracellular matrix interactions.

In an embodiment of the present invention, there is provided a pharmaceutical composition comprising the compound of Formula I as disclosed herein optionally with at least one pharmaceutically acceptable salt thereof, wherein the pharmaceutical composition inhibits, or modulates, or alters, or enhances cell-cell interactions, cell-pathogen interactions, or cell-extracellular matrix interactions either directly or through metabolic processing.

In an embodiment of the present invention, there is provided a pharmaceutical composition comprising the compound selected from Formula II, Formula III, or Formula IV as disclosed herein optionally with at least one pharmaceutically acceptable salt thereof, wherein the pharmaceutical composition inhibits, or modulates, or alters, or enhances cell-cell interactions, cell-pathogen interactions, or cell-extracellular matrix interactions either directly or through metabolic processing

In an embodiment of the present invention, there is provided a pharmaceutical composition comprising the compound of Formula I as disclosed herein optionally with at least one pharmaceutically acceptable salt thereof, wherein the pharmaceutical composition is capable of modulating glycan-protein interactions, glycan-glycan interactions, sialic acids-proteins interactions, plant-pathogen interactions, sialyl-Lewis X/Lewis Y-selectins interactions, sialoglycoconjugates-siglecs interactions, polysialic acid-sialo-lectins interactions, sialic acid-neuraminidases interactions, sialic acid-hemagglutinin interactions, sialic acid-microbial adhesin interactions, gangliosides, or combinations thereof.

In an embodiment of the present invention, there is provided a pharmaceutical composition comprising the compound of Formula I as disclosed herein optionally with at least one pharmaceutically acceptable salt thereof, wherein the pharmaceutical composition is an adjunct in combinatorial cancer chemotherapy or an adjunct in combinatorial cancer immunotherapy.

In an embodiment of the present invention, there is provided a pharmaceutical composition comprising the compound of Formula I as disclosed herein optionally with at least one pharmaceutically acceptable salt thereof for use in method of prevention or treatment or modification of a condition or a disease, the method comprising administering the compound of Formula I as disclosed herein to a subject in need thereof.

In an embodiment of the present invention, there is provided a pharmaceutical composition comprising the compound of Formula I as disclosed herein optionally with at least one pharmaceutically acceptable salt thereof for use in method of prevention or treatment or modification of a condition or a disease selected from acute inflammatory disorders, chronic inflammatory disorders, asthma, allergy, psoriasis, rheumatoid arthritis, tumor metastasis, cancer metastasis, reperfusion syndrome, cytokine storm, viral infectious diseases, bacterial infectious diseases, acute inflammation of liver, neutrophil infiltration, xenograft maintenance, neoplasm, tuberculosis, acute liver injury, acute kidney injury, thrombosis, chronic obstructive pulmonary disease (COPD), hay fever, stroke, atherosclerosis, rhinitis, contact dermatitis, atopic dermatitis, inflammatory bowel disease, multiple sclerosis, type I diabetes, organ transplant rejection, systemic lupus erythematosus (SLE), cystic fibrosis, congenital disorders of glycosylation, leukocyte adhesion deficiency disorders, or fertilization disorder, the method comprising administering the compound of Formula I as disclosed herein to a subject in need thereof.

In an embodiment of the present invention, there is provided a method of prevention or treatment or modification of a condition or a disease, the method comprising administering the compound of Formula I or the pharmaceutical composition as disclosed herein to a subject in need thereof.

In an embodiment of the present invention, there is provided a method of prevention or treatment or modification of a condition or a disease selected from acute inflammatory disorders, chronic inflammatory disorders, asthma, allergy, psoriasis, rheumatoid arthritis, tumor metastasis, cancer metastasis, reperfusion syndrome, cytokine storm, viral infectious diseases, bacterial infectious diseases, acute inflammation of liver, neutrophil infiltration, xenograft maintenance, neoplasm, tuberculosis, acute liver injury, acute kidney injury, thrombosis, chronic obstructive pulmonary disease (COPD), hay fever, stroke atherosclerosis, rhinitis, contact dermatitis, atopic dermatitis, inflammatory bowel disease, multiple sclerosis, type I diabetes, organ transplant rejection, systemic lupus erythematosus (SLE), cystic fibrosis, congenital disorder of glycosylation, leukocyte adhesion deficiency disorders, or fertilization disorder, the method comprising administering the compound of Formula I or the pharmaceutical composition as disclosed herein to a subject in need thereof.

In an embodiment of the present invention, there is provided a method for engineering cell surface epitopes, the method comprising: contacting a cell with a compound of Formula I or the pharmaceutical composition as disclosed herein.

In an embodiment of the present invention, there is provided a method for engineering cell surface epitopes, the method comprising: contacting a cell with a compound selected from the compound of Formula II, Formula III, or Formula IV or its pharmaceutical composition as disclosed herein.

In an embodiment of the present invention, there is provided a method for engineering of glycan epitopes on secreted glycoproteins such as, but not limited to, monoclonal antibodies, growth factors, cytokines, chemokines, biopharmaceuticals, and biologics, in a mammalian bioreactor system, the method comprising: contacting a cell with a compound of Formula I or the pharmaceutical composition as disclosed herein.

In an embodiment of the present invention, there is provided a method for engineering cell surface epitopes, the method comprising: contacting a cell with a compound of Formula I or the pharmaceutical composition as disclosed herein through metabolic biosynthetic pathways.

In an embodiment of the present invention, there is provided a method for engineering cell surface epitopes, the method comprising: contacting a cell with a compound of Formula I or the pharmaceutical composition as disclosed herein, wherein the cell surface epitope is glycan epitope selected from sialic acid epitopes, including partially acetylated sialoglycan epitopes (9-O—Ac, 7-O—Ac, 8-O-Ac, 4-O—Ac individually or in combinations), sialyl-Lewis-A epitope, sialyl-Lewis-X epitope, sulfo-sialyl-Lewis-X epitope, Lewis-X(CD15)/Y/A/B epitope, Sialyl-TF (Thomson-Friedenreich) antigen, ganglioslides, or polysialicacid epitopes.

In an embodiment of the present invention, there is provided a compound of Formula I as disclosed herein, wherein the compound of Formula I is capable of modulating sialoglycans, that are attached to proteins, polypeptides, glycolipids, and RNA, interacting with viral neuraminidases and hemagglutinins, sialoglycans interacting with bacterial adhesions, TF-antigen interactions with galectins, Tn-antigen (GalNAc-α-Ser/Thr) with endogenous and exogenous lectins, galactoglycans interacting with bacterial, parasite, and plant lectins, β-O-GlcNAc modified nuclear/cytoplasmic proteins, including transcription factors, interacting with DNA/RNA and adapter proteins, or glycoconjugate interactions with glycosaminoglycans and proteoglycans.

In an embodiment of the present invention, there is provided a pharmaceutical composition comprising the compound of Formula I as disclosed herein optionally with at least one pharmaceutically acceptable salt thereof, wherein the pharmaceutical composition is capable of modifying sialoglycans interacting with viral neuraminidases and hemagglutinins, sialoglycans interacting with bacterial adhesions, TF-antigen interactions with galectins, Tn-antigen (GalNAc-α-Ser/Thr) with endogenous and exogenous lectins, galactoglycans interacting with bacterial, parasite, and plant lectins, β-O-GlcNAc modified nuclear/cytoplasmic proteins, including transcription factors, interacting with DNA/RNA and adapter proteins, or glycoconjugate interactions with glycosaminoglycans and proteoglycans.

In an embodiment of the present invention, there is provided a use of the compound of Formula I as disclosed herein for prevention or treatment or modification of a condition or a disease selected from acute inflammatory disorders, chronic inflammatory disorders, asthma, allergy, psoriasis, rheumatoid arthritis, tumor metastasis, cancer metastasis, reperfusion syndrome, cytokine storm, viral infectious diseases, bacterial infectious diseases, acute inflammation of liver, neutrophil infiltration, xenograft maintenance, neoplasm, tuberculosis, acute liver injury, acute kidney injury, thrombosis, chronic obstructive pulmonary disease (COPD), hay fever, stroke, atherosclerosis, rhinitis, contact dermatitis, atopic dermatitis, inflammatory bowel disease, multiple sclerosis, type I diabetes, organ transplant rejection, systemic lupus erythematosus (SLE), cystic fibrosis, congenital disorders of glycosylation, leukocyte adhesion deficiency disorder, or fertilization disorder.

In an embodiment of the present invention, there is provided a use of a compound selected from the compound of Formula II, Formula III or Formula IV as disclosed herein for prevention or treatment or modification of a condition or a disease selected from acute inflammatory disorders, chronic inflammatory disorders, asthma, allergy, psoriasis, rheumatoid arthritis, tumor metastasis, cancer metastasis, reperfusion syndrome, cytokine storm, viral infectious diseases, bacterial infectious diseases, acute inflammation of liver, neutrophil infiltration, xenograft maintenance, neoplasm, tuberculosis, acute liver injury, acute kidney injury, thrombosis, chronic obstructive pulmonary disease (COPD), hay fever, stroke, atherosclerosis, rhinitis, contact dermatitis, atopic dermatitis, inflammatory bowel disease, multiple sclerosis, type I diabetes, organ transplant rejection, systemic lupus erythematosus (SLE), cystic fibrosis, congenital disorders of glycosylation, leukocyte adhesion deficiency disorder, or fertilization disorder.

In an embodiment of the present invention, there is provided use of the compound of Formula I or the pharmaceutical composition as disclosed herein, for engineering cell surface epitopes by contacting a cell with the compound of Formula I or its pharmaceutical composition, wherein the cell surface epitope is glycan epitope selected from sialic acid epitope, sialyl-Lewis-A epitope, sialyl-Lewis-X epitope, sulfo-sialyl-Lewis-X epitope, Lewis-X(CD15)/Y/A/B epitope, Sialyl-TF (Thomson-Friedenreich) antigen, gangliosides, or polysialicacid epitopes.

In an embodiment of the present invention, there is provided use of the compound of Formula I or the pharmaceutical composition as disclosed herein, for engineering cell surface epitopes by contacting a cell with the compound of Formula I or its pharmaceutical composition through metabolic biosynthetic pathways, wherein the cell surface epitope is glycan epitope selected from sialic acid epitope, sialyl-Lewis-A epitope, sialyl-Lewis-X epitope, sulfo-sialyl-Lewis-X epitope, Lewis-X(CD15)/Y/A/B epitope, Sialyl-TF (Thomson-Friedenreich) antigen, gangliosides, or polysialicacid epitopes

In an embodiment of the present invention, there is provided use of the compound selected from a compound of Formula II, Formula III, or Formula IV, or its pharmaceutical composition as disclosed herein, for engineering cell surface epitopes by contacting a cell with the compound of Formula I or its pharmaceutical composition, wherein the cell surface epitope is glycan epitope selected from sialic acid epitope, sialyl-Lewis-A epitope, sialyl-Lewis-X epitope, sulfo-sialyl-Lewis-X epitope, Lewis-X(CD15)/Y/A/B epitope, Sialyl-TF (Thomson-Friedenreich) antigen, gangliosides, or polysialicacid epitopes.

In an embodiment of the present invention, there is provided use of the compound selected from a compound of Formula II, Formula III, or Formula IV, or its pharmaceutical composition as disclosed herein, for engineering cell surface epitopes by contacting a cell with the compound of Formula I or its pharmaceutical composition through metabolic biosynthetic pathways, wherein the cell surface epitope is glycan epitope selected from sialic acid epitope, sialyl-Lewis-A epitope, sialyl-Lewis-X epitope, sulfo-sialyl-Lewis-X epitope, Lewis-X(CD15)/Y/A/B epitope, Sialyl-TF (Thomson-Friedenreich) antigen, gangliosides, or polysialicacid epitopes

Although the subject matter has been described in considerable detail with reference to certain examples and implementations thereof, other implementations are possible.

### EXAMPLES

The following examples provide the details about the synthesis, activities, and applications of the compounds of the present disclosure. It should be understood the following is representative only, and that the invention is not limited by the details set forth in these examples.

### Materials and Methods:

All solvents, chemical reagents, and monosaccharides were procured from commercial sources (Sigma-Aldrich, Merck, and New Zealand Pharmaceuticals). Hexosamine starting materials, namely, D-Mannosamine hydrochloride, D-glucosamine hydrochloride, D-galactosamine hydrochloride, reagents, and solvents were purchased commercially. Compounds were purified by flash column chromatography using silica gel 60 (230-400 mesh) manually. Solvents used for chromatography were either analytical grade or distilled prior to use. Solvent evaporations were performed using rotary evaporator. Thin layer chromatography (TLC) was performed using fluorescent silica gel glass plates (Analtech/Miles Scientific) and visualised using handheld UV lamp and mostain. NMR (¹H and ¹³C) was recorded on a Bruker 300 MHz spectrometer using chloroform-d, methanol-d4, dimethyl sulfoxide-d6, and deuterium oxide as solvents with tetramethylsilane (TMS) as internal reference. Mass spectrometry was performed using high resolution ESI-MS (Thermo Orbitrap Velos) or MALDI-TOF-TOF (AB Sciex 4800) instruments. For MALDI, super-DHB was used as matrix.

### General Preparation of Compounds of Formula I

To a solution or suspension of the selective protected hexosamine salts (1.0 mmol) (Formula A), the carboxylic acid (cycloalkylcarboxylic acid or cycloalkylaceticacid) (2.0-3.0 mmol), and 1-ethyl-3-(3-dimethylaminopropyl)carbodiimide hydrochloride (EDC·HCl) (2.0-3.0 mmol) in dimethylformamide (DMF) or dioxan (50 mL) at 25° C. was added with triethylamine (5.0-10 mmol) under vigorous stirring for 12-24 h. The reaction mixture was concentrated and the residue was extracted with dichloromethane (100 mL) and saturated aqueous sodium bicarbonate solution (100 mL) in a 250 mL separatory funnel. The organic layer was separated, dried over anhydrous Na₂SO₄, filtered and concentrated. The residue was purified by silica gel column chromatography using ethyl acetate and hexanes as the eluant to obtain the respective Formula II (ManNAc), Formula III (GalNAc), and Formula IV (GlcNAc) compounds. The purified products were characterized using thin layer chromatography (TLC), Nuclear magnetic resonance (NMR) spectroscopy (both proton and carbon-13), and high resolution mass spectrometry.

### Example 1

### Synthesis of Compounds of Formula II (ManNAc Series of Compounds)

For the synthesis of ManNAc series of compounds, the 1,3,4,6-tetra-O-acetyl-D-α/β-mannopyranose oxalic acid salt (Ac₄ManNH₃⁺Ox⁻), prepared according to the previously reported procedure (Sampathkumar, S. G., Li, A. V., Yarema, K. J., Nat Protoc 1, 2377-2385(2006)), was employed as the starting material.

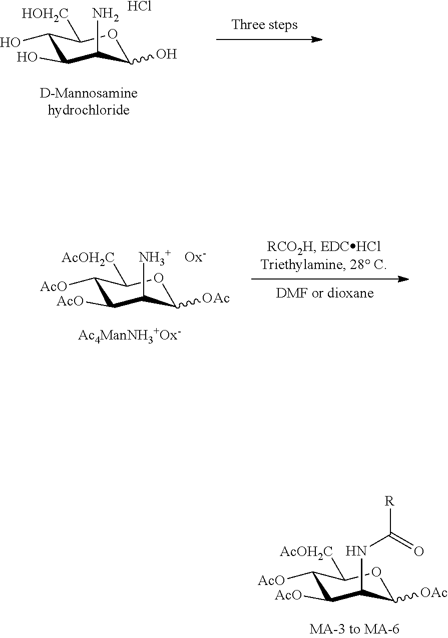

To Ac₄ManNH₃⁺Ox⁻ (Formula A, 1.0 mmol) the corresponding cycloalkylcarboxylic acid or cycloalkylaceticacid (3.0 mmol), and EDC. HCl (2.0 mmol) in DMF (20 mL) was treated, at 25° C., with triethylamine (6.0 mmol) with stirring. After 16 h, the reaction mixture was concentrated, the residue was dissolved in dichloromethane (100 mL) and washed with aq. NaHCO₃ (5% w/v) in a separatory funnel. The organic layer was separated, dried (anh. Na₂SO₄), filtered, and concentrated. The products were purified using silica gel 60 (mesh size 260-400) column chromatography using hexanes/ethyl acetate as the eluant. The products were characterized by TLC, NMR (¹H and ¹³C), and MALDI-TOF or ESI mass spectrometry.

In an example (3S,4R,5S,6R)-6-(acetoxymethyl)-3-(2-cyclopentylacetamido)tetrahydro-2H-pyran-2,4,5-triyl triacetate/Acetyl 3,4,6-tri-O-acetyl-2-cyclopentylacetamido-2-deoxy-α-D-manno-hexopyranoside (compound xvi) was prepared by the method as explained herein. The solution of Ac₄ManNH₃.Oxalate (SP IV/01) (Formula A, 0.25 g, 0.5717 mmol, 1 eq) and EDC.HCl (0.22 g, 1.143 mmol, 2 eq) in 15 mL DMF was stirred at room temperature. A clear yellowish solution obtained. Then, cyclopentylacetic acid (0.147 g, 1.143 mmol, 2 eq) was added dropwise to the reaction mixture followed by the addition of NEt₃ (220 μL, mmol). The reaction mixture was continued to stir for 24 h at room temperature. After stirring, the reaction mixture was analysed by TLC. The reaction mixture was concentrated, the residue thus obtained was dissolved in 50 mL CH₂Cl₂ and washed with 50 mL NaHCO₃ solution (2% w/v %). The aqueous layer was again washed twice with CH₂Cl₂ (25 mL). Organic layers were combined, dried over anhydrous Na₂SO₄, filtered and concentrated. The resulting residue was purified with Column chromatography in H:E (10% to 100% E) to obtain pure compound xvi (15 mg, 5.7%). Molecular formula: C₂₁H₃₁NO₁₀

¹H NMR (300 MHz, CDCl₃) δ 6.01 (d, 1H, J=3.0 Hz), 5.72 (d, 1H, J=9.0 Hz), 5.32 (dd, 1H, J=9.0 Hz), 5.18 (t, 1H, J=9.0 Hz), 4.65 (ddd, 1H), 4.28 (dd, 1H, J=12 Hz), 4.07-4.01 (m, 1H), 2.25 (s, 2H), 2.17 (s, 3H), 2.09 (s, 3H), 2.05 (s, 3H), 1.99 (s, 3H), 1.88-1.84 (broad, 1H), 1.707-1.528 (broad, 5H), 1.25-1.19 (broad, 4H).

### Synthesis of Compounds of Formula III (GalNAc Series of Compounds)

For the synthesis of GalNAc series of compounds, the 1,3,4,6-tetra-O-acetyl-D-α/β-galactopyranose hydrochloride (Ac₄GalNH3⁺Cl⁻), prepared according to a reported procedure (Kim, T. Y., Davidson, E. A., J. Org. Chem. 28, 2475 (1963)), was employed as the starting material.

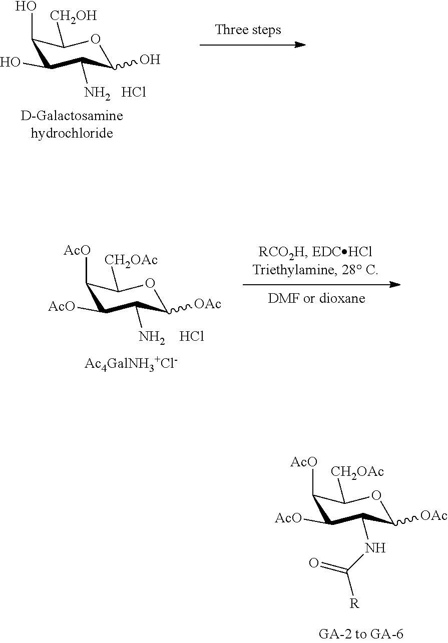

To Ac₄GalNH3⁺Cl⁻ (Formula A, 1.0 mmol) the corresponding cycloalkylcarboxylic acid or cycloalkylaceticacid (3.0 mmol), and EDC. HCl (2.0 mmol) in DMF (20 mL) was treated, at 25° C., with triethylamine (6.0 mmol) with stirring. After 16 h, the reaction mixture was concentrated, the residue was dissolved in dichloromethane (100 mL) and washed with aq. NaHCO₃ (5% w/v) in a separatory funnel. The organic layer was separated, dried (anh. Na₂SO₄), filtered, and concentrated. The products were purified using silica gel 60 (mesh size 260-400) column chromatography using hexanes/ethyl acetate as the eluant. The product were characterized by TLC, NMR (¹H and ¹³C), and MALDI-TOF or ESI mass spectrometry.

In an example (3R,4R,5R,6R)-6-(acetoxymethyl)-3-(cyclohexanecarboxamido)tetrahydro-2H-pyran-2,4,5-triyl triacetate (viii) compound was prepared by treating solution of Ac₄GalNH₂·HCl (JR II/60) (Formula A, 0.5 g, 1.3037 mmol, 1 eq) and EDC.HCl (0.5 g, 2.60 mmol, 2 eq) in 25 mL dioxane was stirred at room temperature. A white suspension was obtained. Then, cyclohexane carboxylic acid (485 μL, 3.91 mmol, 3 equiv.) was added to the reaction mixture followed by the addition of NEt₃ (18201 μL, 13.04 mmol, 10 equiv.) and DMAP (catalytic amount). Then, the reaction mixture was continued to stir for 24 h at room temperature. After stirring, the reaction mixture was analysed by TLC. The reaction mixture was concentrated, the residue thus obtained was dissolved in 50 mL CH₂Cl₂ and washed with 50 mL NaHCO₃ solution (5% w/v %). The aqueous layer was again washed with CH₂Cl₂ (50 mL). Organic layers were combined, dried over anhydrous Na₂SO₄, filtered and concentrated. The resulting residue was purified with Column chromatography in H:E (15% to 100% E) to obtain pure compound viii (0.15 g, 25%). Molecular Formula: C₂₁H₃₁NO₁₀.

¹H NMR (300 MHz, CDCl₃) δ 5.70 (d, 1H, J=9.0 Hz), 5.465 (d, 1H, J=9.0 Hz), 5.36 (dd, 1H, J=3.0 Hz), 5.095 (dd, 1H, J=12 Hz), 4.47 (dt, 1H), 4.20-4.07 (m, 2H), 4.02 (td, 1H, J=6.0 Hz), 2.16 (s, 3H), 2.09 (s, 3H), 2.04 (s, 3H), 1.99 (s, 3H), 1.77-1.68 (m, 5H), 1.38-1.18 (m, 6H). ¹³C NMR (75 Hz, CDCl₃):176.39, 170.72, 170.46, 170.23, 169.57, 93.10, 71.95, 70.28, 66.46, 61.41, 49.32, 45.59, 29.50, 29.43, 25.55, 25.52, 20.83, 20.68, 20.61

In one another example, (3R,4R,5R,6R)-6-(acetoxymethyl)-3-(2-cyclopentylacetamido)tetrahydro-2H-pyran-2,4,5-triyl triacetate/Acetyl 3,4,6-tri-O-acetyl-2-cyclopentylacetamido-2-deoxy-b-D-galacto-hexopyranoside (compound xxi) was prepared by the process as defined herein. The solution of Ac₄GalNH₂·HCl (JR II/60) (Formula A, 100 mg, 0.26 mmol, 1.0 equiv.) and EDC.HCl (100 mg, 0.52 mmol, 2 eq) in 10 mL dioxane was stirred at room temperature. A white suspension obtained. Then, cyclopentylacetic acid (67 mg, 0.52 mmol, 2 equiv.) was added to the reaction mixture followed by the dropwise addition of NEt₃ (182 μL, 1.3 mmol, 05 equiv.). Then, the reaction mixture was continued to stir for 24 h at room temperature. After stirring, the reaction mixture was analysed by TLC. The reaction mixture was concentrated, the residue thus obtained was purified with Column chromatography in H:E (10% to 60% E) to obtain pure compound xxi (30 mg, 25%). Molecular Formula: C₂₁H₃₁NO₁₀.

¹H NMR (300 MHz, CDCl₃) δ 5.70 (d, 1H, J=9.0 Hz), 5.37 (m, 1H, J=3.0 Hz), 5.09 (dd, 1H, J=12 Hz, 9.0 Hz), 4.47 (dt, 1H), 4.21-4.08 (m, 3H), 4.01 (dt, 1H, J=6.0 Hz), 2.17 (s, 2H), 2.13 (s, 3H), 2.12 (s, 3H), 2.04 (s, 3H), 2.00 (s, 3H), 1.76-1.72 (broad, 1H), 1.61(s), 1.585-1.469 (m, 4H), 1.08-1.07 (m, 4H). HR-ESI-MS Calcd for C₂₁I₃₁NO₁₀+Na⁺480.1840; found 480.1830

### Synthesis of Compounds of Formula IV (GlcNAc Series of Compounds)

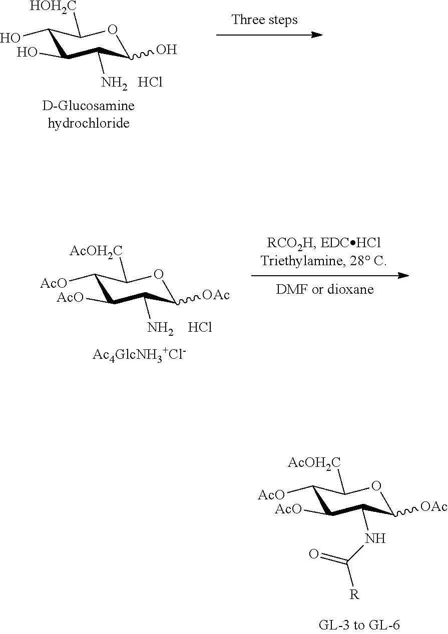

For the synthesis of GlcNAc series of compounds, the 1,3,4,6-tetra-O-acetyl-D-α/β-glucopyranose hydrochloride (Ac₄GlcNH₃⁺Cl⁻), prepared according to a reported procedure (Medgyes, A., Farkas, E., Liptak, A., Pozsgay, V. Tetrahedron, 4159 (1997)), was employed as the starting material.

To Ac₄GlcNH₃⁺Cl⁻ (Formula A, 1.0 mmol), the corresponding cycloalkylcarboxylic acid or cycloalkylaceticacid (3.0 mmol), and EDC. HCl (2.0 mmol) in DMF (20 mL) was treated, at 25° C., with triethylamine (6.0 mmol) with stirring. After 16 h, the reaction mixture was concentrated; the residue was dissolved in dichloromethane (100 mL), and washed with aq.NaHCO₃ (5% w/v) in a separatory funnel. The organic layer was separated, dried (anh. Na₂SO₄), filtered, and concentrated. The products were purified using silica gel 60 (mesh size 260-400) column chromatography using hexanes/ethyl acetate as the eluant. The products were characterized by TLC, NMR (¹H and ¹³C), and MALDI-TOF or ESI mass spectrometry.

For comparative purposes the compounds 1a, 1c, 1d, 1e, 1f, 1g, 2a, and 3 as illustrated below were prepared by known methods.

Compound 1a—1,3,4,6-tetra-O-acetyl-2-cyclopropanoylamino-2-deoxy-α-D-mannopyranose; and Compound 2a—1,3,4,6-tetra-O-acetyl-2-cyclopropanoylamino-2-deoxy-β-D-glucopyranose (Hassenruck, J. & Wittmann, V. Cyclopropene derivatives of aminosugars for metabolic glycoengineering. Beilstein J Org Chem 15, 584-601 (2019)).

Compound 1c—1,3,4,6-tetra-O-acetyl-2-azidoacetylamino-2-deoxy-α-D-mannopyranose; Compound 1d—1,3,4,6-tetra-O-acetyl-2-acetylamino-2-deoxy-α/β-D-mannopyranose; Compound 1e —1,3,4,6-tetra-O-acetyl-2-(1-propanoyl)amino-2-deoxy-α-D-mannopyranose; Compound 1f—1,3,4,6-tetra-O-acetyl-2-(1-butanoyl)amino-2-deoxy-α-D-mannopyranose; and Compound 1g —1,3,4,6-tetra-O-acetyl-2-(1-pentanoyl)amino-2-deoxy-D-mannopyranose (Saxon, E. & Bertozzi, C. R. Cell surface engineering by a modified Staudinger reaction, Science 287, 2007-2010 (2000); Shajahan, A. et al. Carbohydrate-Neuroactive Hybrid Strategy for Metabolic Glycan Engineering of the Central Nervous System in Vivo, J Am Chem Soc 139, 693-700 (2017); Jacobs, C. L. et al. Substrate specificity of the sialic acid biosynthetic pathway, Biochemistry 40, 12864-12874 (2001); and Kim, E. J. et al. Characterization of the metabolic flux and apoptotic effects of O-hydroxyl- and N-acyl-modified N-acetylmannosamine analogs in Jurkat cells, J Biol Chem 279, 18342-18352 (2004)).

Compound 3—1,3,4,6-tetra-O-acetyl-2-azidoacetylamino-2-deoxy-β-D-galactopyranose (Hang, H. C., et al., A metabolic labeling approach toward proteomic analysis of mucin-type O-linked glycosylation, Proc Natl Acad Sci U S A 100, 14846-14851 (2003))

Table 1 and Table 2 below depict the compounds of Formula II, III and IV synthesized by the processes explained above.

TABLE 1

| S. | Compound | | Characterization |
|---|---|---|---|
| No. | Structure | Compound name | details |
| | Formula II (MA Series) | | |
| i | 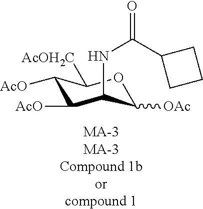 | (3S,4R,5S,6R)-6- (acetoxymethyl)-3- (cyclobutanecarboxamido) tetrahydro-2H- pyran-2,4,5-triyl triacetate/Acetyl 3,4,6-tri-O-acetyl-2- cyclobutanecarboxamido- 2-deoxy-α/β-D- manno- hexopyranoside | Yield: 45% (110 mg) ¹H NMR (300 MHz, CDCl₃) δ 6.01 (d, J = 1.8 Hz, 1H), 5.53 (d, J = 9.3 Hz, 1H), 5.32 (dd, J = 10.2 Hz, 4.5 Hz, 1H), 5.12 (dd, J = 10.2 Hz, 10.2 Hz, 1H), 4.65 (ddd, J = 9.3 Hz, 4.5 Hz, 1.8 Hz, 1H), 4.25 (dd, J = 12.3 Hz, 4.5 Hz, 1H), 4.05 (dd, J = 12.3 Hz, 2.4 Hz, 1H), |
| | | | 4.03 (ddd, J = 8.4, Hz, |
| | | | 4.2 Hz, 2.4 Hz, 1H), |
| | | | 3.06 (q, J = 8.4 Hz, |
| | | | 1H), 2.27 (m, 4H), 2.17 |
| | | | (s, 3H), 2.09 (s, 3H), |
| | | | 2.06 (s, 3H), 2.00 (s, |
| | | | 3H), 1.96 (m, 2H). |
| | | | ¹³C NMR (75 MHz, |
| | | | CDCl₃) 175.0, 170.5, |
| | | | 170.0 169.7, 168.1, |
| | | | 91.8, 70.1, 68.9, 65.4, |
| | | | 62.0, 49.0, 39.7, 25.32, |
| | | | 25.3, 20.97, 20.8, 20.7, |
| | | | 20.6, 18.2. ESI MS m/z |
| | | | Calcd for |
| | | | [C₁₉H₂₇NO₁₀ + Na]⁺ |
| | | | 452.1533; Found |
| | | | 452.1527. |
| ii | 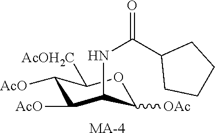 | (3S,4R,5S,6R)-6- (acetoxymethyl)-3- (cyclopentanecarboxamido) tetrahydro-2H- pyran-2,4,5-triyl triacetate/Acetyl 3,4,6-tri-O-acetyl-2- cyclopentanecarboxamido- 2-deoxy-α/β-D- | ¹H NMR (300 MHz, CDCl₃) δ 6.00 (d, J = 1.8 Hz, 1H), 5.77 (d, J = 9.3 Hz, 1H), 5.29 (dd, J = 9.9 Hz, 4.5 Hz, 1H), 5.15 (dd, J = 10.2 Hz, 9.9 Hz, 1H), 4.64 (ddd, J = 9.3 Hz, 4.5 Hz, 1.8 Hz, 1H), |
| | | manno- | 4.24 (dd, J = 9.6 Hz, |
| | | hexopyranoside | 5.1 Hz, 1H), 4.04 (dd, |
| | | | J = 11.7 Hz, 2.7 Hz, |
| | | | 1H), 4.03 (ddd, J = |
| | | | 8.7 Hz, 4.8 Hz, 2.7 Hz |
| | | | 1H), 2.59 (q, J = 7.5 Hz, |
| | | | 1H), 2.15 (s, 3H), |
| | | | 2.07 (s, 3H), 2.04 (s, |
| | | | 3H), 1.96 (s, 3H), 1.95- |
| | | | 1.52 (m, 8H). |
| | | | ¹³C NMR (75 MHz, |
| | | | CDCl₃) δ 176.4, |
| | | | 170.5, 169.9, 169.7, |
| | | | 168.1, 91.8, 70.1, |
| | | | 69.0, 65.5, 62.1, 49.0, |
| | | | 45.6, 30.6, 30.2, |
| | | | 25.91, 25.88, 20.82, |
| | | | 20.79, 20.64, 20.61. |
| iii | 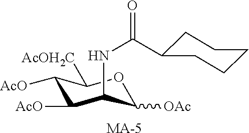 | (3S,4R,5S,6R)-6- (acetoxymethyl)-3- (cyclohexanecarboxamido) tetrahydro-2H- pyran-2,4,5-triyl triacetate Acetyl 3,4,6- tri-O-acetyl-2- cyclohexanecarboxamido- 2-deoxy-α/β-D- | |
| | | manno- | |
| | | hexopyranoside | |
| iv | 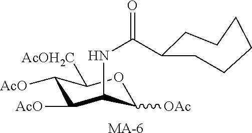 | (3S,4R,5S,6R)-6- (acetoxymethyl)-3- (cycloheptanecarboxamido) tetrahydro-2H- pyran-2,4,5-triyl triacetate/Acetyl 3,4,6-tri-O-acetyl-2- cycloheptanecarboxamido- 2-deoxy-α/β-D- | |
| | | manno- | |
| | | hexopyranoside | |
| | Formula III (GA Series) | | |
| v | 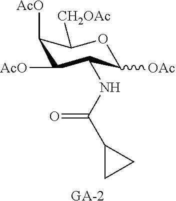 | (3R,4R,5R,6R)-6- (acetoxymethyl)-3- (cyclopropanecarboxamido) tetrahydro-2H- pyran-2,4,5-triyl triacetate/Acetyl 3,4,6- tri-O-acetyl-2- cyclopropanecarboxamido- 2-deoxy-α/β-D- galacto-hexopyranoside | Yield: 77% (830 mg) ¹H NMR (300 MHz, CDCl₃) δ 5.86 (d, J = 9.6 Hz, 1H), 5.72 (d, J = 8.7 Hz, 1H), 5.36 (d, J = 3.0 Hz, 1H), 5.11 (dd, J = 11.4 Hz, 3.3 Hz, 1H), 4.47 (ddd, J = 9.3 Hz, 1H), 4.18- 4.02 (m, 3H), 2.14 (s, 3H), 2.10 (s, 3H), 2.03 (s, 3H), 1.99 (s, 3H), |
| | | | 1.28 (m, 1H), 0.91 (m, |
| | | | 2H), 0.72 (m, 2H). |
| | | | ¹³C NMR (75 MHz, |
| | | | CDCl₃) δ 174.1, |
| | | | 170.8, 170.5, 170.3, |
| | | | 169.6, 93.1, 72.0, |
| | | | 70.4, 66.5, 61.4, 49.7, |
| | | | 20.8, 20.7, 20.6 (2C), |
| | | | 14.7, 7.5, 7.4. ESI MS |
| | | | m/z Calcd for |
| | | | [C₁₈H₂₅NO₁₀ + Na]⁺ |
| | | | 438.1376. Found |
| | | | 438.1370. |
| vi | 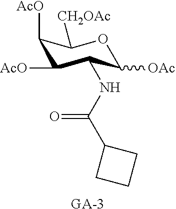 | (3R,4R,5R,6R)-6- (acetoxymethyl)-3- (cyclobutanecarboxamido) tetrahydro-2H- pyran-2,4,5-triyl triacetate/Acetyl 3,4,6-tri-O-acetyl-2- cyclobutanecarboxamido- 2-deoxy-α/β-D- galacto- hexopyranoside | Yield: 75% (836 mg) ¹H NMR (300 MHz, CDCl₃) δ 5.70 (d, J = 8.7 Hz, 1H), 5.46 (d, J = 9.6 Hz, 1H), 5.35 (d, J = 3.0 Hz, 1H), 5.09 (dd, J = 11.4 Hz, 3.3 Hz, 1H), 4.45 (ddd, J = 9.3 Hz, 1H), 4.19- 4.00 (m, 3H), 2.91 (q, J = 8.4 Hz, 1H), 2.16- 2.04 (m, 4H), 2.15 (s, 3H), 2.10 (s, 3H), |
| | | | 2.03 (s, 3H), 1.98 (s, |
| | | | 3H), 1.80-1.40 (m, |
| | | | 2H). |
| | | | ¹³C NMR (75 MHz, |
| | | | CDCl₃) δ 175.3, |
| | | | 170.7, 170.5, 170.2, |
| | | | 169.6, 93.1, 71.9, |
| | | | 70.4, 66.4, 61.4, 49.5, |
| | | | 39.8, 36.6, 25.2, 24.7, |
| | | | 20.8, 20.65 |
| | | | (2C), 18.2. ESI MS m/z |
| | | | Calcd for |
| | | | [C₁₉H₂₇NO₁₀ + Na]⁺ |
| | | | 452.1533; Found |
| | | | 452.1527 |
| vii | 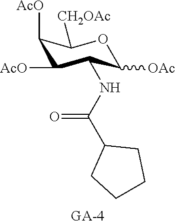 | (3R,4R,5R,6R)-6- (acetoxymethyl)-3- (cyclopentanecarboxamido) tetrahydro-2H- pyran-2,4,5-triyl triacetate/Acetyl 3,4,6- tri-O-acetyl-2- cyclopentanecarboxamido- 2-deoxy-α/β-D- galacto-hexopyranoside | Yield: 65% (752 mg) ¹H NMR (300 MHz, CDCl₃) δ 5.70 (d, J = 8.7 Hz, 1H), 5.38 (d, J = 8.7 Hz, 1H), 5.37 (d, J = 2.1 Hz, 1H), 5.09 (dd, J = 11.4 Hz, 3.3 Hz, 1H), 4.48 (ddd, J = 9.3 Hz, 1H), 4.21- 4.08 (m, 2H), 4.01 (dt, J = 6.6 Hz, 1.2 Hz, 1H), 2.43 (q, J = 7.5 Hz, 1H), 2.16 (s, 3H), 2.11 (s, 3H), 2.04 (s, |
| | | | 3H), 2.00 (s, 3H), 1.80- |
| | | | 1.54 (m, 8H). ¹³C |
| | | | NMR (75 MHz, |
| | | | CDCl₃) δ 176.4, |
| | | | 170.7, 170.4, 170.1, |
| | | | 169.5, 93.2, 72.0, |
| | | | 70.4, 66.4, 61.3, 49.6, |
| | | | 45.9, 30.35, 30.28, |
| | | | 25.8 (2C), 20.8, 20.63 |
| | | | 20.57. MALDI-TOF |
| | | | m/z Calcd for |
| | | | [C₂₀H₂₉NO₁₀ + Na]⁺ |
| | | | 466.1689; Found |
| | | | 466.1346. |
| viii | 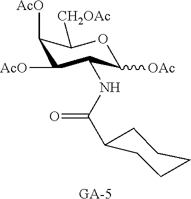 | (3R,4R,5R,6R)-6- (acetoxymethyl)-3- (cyclohexanecarboxamido) tetrahydro-2H- pyran-2,4,5-triyl triacetate/Acetyl 3,4,6-tri-O-acetyl-2- cyclohexanecarboxamido- 2-deoxy-α/β-D- galacto- hexopyranoside | 1H NMR (300 MHz, CDCl₃) δ 5.70 (d, 1H, J = 9.0 Hz), 5.465 (d, 1H, J = 9.0 Hz), 5.36 (dd, 1H, J = 3.0 Hz), 5.095 (dd, 1H, J = 12 Hz), 4.47 (dt, 1H), 4.20-4.07 (m, 2H), 4.02 (td, 1H, J = 6.0 Hz), 2.16 (s, 3H), 2.09 (s, 3H), 2.04 (s, 3H), 1.99 (s, 3H), |
| | | | 1.77-1.68 (m, 5H), |
| | | | 1.38-1.18 (m, 6H). |
| | | | ¹³C NMR (75 Hz, |
| | | | CDCl₃): 176.39, |
| | | | 170.72, 170.46, |
| | | | 170.23, 169.57, 93.10, |
| | | | 71.95, 70.28, 66.46, |
| | | | 61.41, 49.32, 45.59, |
| | | | 29.50, 29.43, 25.55, |
| | | | 25.52, 20.83, 20.68, |
| | | | 20.61 |
| ix | 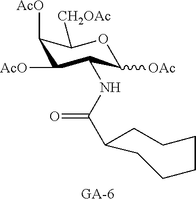 | (3R,4R,5R,6R)-6- (acetoxymethyl)-3- (cycloheptanecarboxamido) tetrahydro-2H- pyran-2,4,5-triyl triacetate/Acetyl 3,4,6-tri-O-acetyl-2- cycloheptanecarboxamido- 2-deoxy-α/β-D- galacto- hexopyranoside | |
| | Formula IV (GlcNAc Series) | | |
| x | 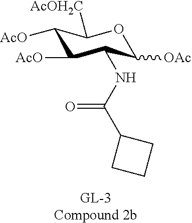 | (3R,4R,5S,6R)-6- (acetoxymethyl)-3- (cyclobutanecarboxamido) tetrahydro-2H- pyran-2,4,5-triyl triacetate/Acetyl 3,4,6-tri-O-acetyl-2- cyclobutanecarboxamido- 2-deoxy-α/β-D- gluco-hexopyranoside | Yield: 92% (1.02 g) ¹H NMR (300 MHz, CDCl₃) δ 5.68 (d, J = 8.7 Hz, 1H), 5.53 (d, J = 9.6 Hz, 1H), 5.18 (dd, J = 9.3 Hz, 9.3 Hz, 1H), 5.13 (dd, J = 9.3 Hz, 9.3 Hz, 1H), 4.37- 4.26 (m, 1H), 4.25 (dd, J = 12.6 Hz, 4.8 Hz, 1H), 4.12 (dd, J = 12.6 Hz, 2.4 Hz, 1H), 3.80 (ddd, J = 8.7 Hz, 4.2 Hz, |
| | | | 2.7 Hz, 1H), 2.90 |
| | | | (q, J = 8.4 Hz, 1H), |
| | | | 2.20-1.70 (m, 6H), |
| | | | 2.09 (s, 3H), 2.08 (s, |
| | | | 3H), 2.03 (s, 3H), 2.02 |
| | | | (s, 3H). |
| | | | ¹³C NMR (75 MHz, |
| | | | CDCl₃) δ 175.0, |
| | | | 171.2, 170.7, 169.5, |
| | | | 169.3, 92.7, 72.9, 72.6, |
| | | | 67.8, 61.7, 52.7, 39.8, |
| | | | 25.2 (2C), 20.8, 20.7, |
| | | | 20.62, 20.57, |
| | | | 18.11. ESI MS m/z |
| | | | Calcd for |
| | | | [C₁₉H₂₇NO₁₀ + Na]⁺ |
| | | | 452.1533; Found |
| | | | 452.1527. |
| xi | 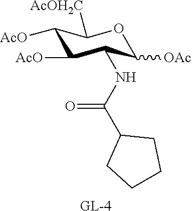 | (3R,4R,5S,6R)-6- (acetoxymethyl)-3- (cyclopentanecarboxamido) tetrahydro-2H- pyran-2,4,5-triyl triacetate/Acetyl 3,4,6-tri-O-acetyl-2- cyclopentanecarboxamido- 2-deoxy-α/β-D- gluco-hexopyranoside | Yield: 81% (930 mg) ¹H NMR (300 MHz, CDCl₃) δ 5.70 (d, J = 9.0 Hz, 1H), 5.67 (d, J = 9.6 Hz, 1H), 5.15 (dd, J = 7.5 Hz, 7.5 Hz, 1H), 5.12 (dd, J = 9.3 Hz, 9.3 Hz, 1 H), 4.32 (m, 1H), 4.25 (dd, J = 12.6 Hz, 4.8 Hz, 1H), 4.11 (dd, J = 12.6 Hz, 2.1 Hz, 1H) 3.81 (ddd, J = 9.3, 4.5, |
| | | | 2.1 Hz, 1H), 2.42 (q, J = |
| | | | 7.8 Hz, 1H), 2.09 (s, |
| | | | 3H), 2.08 (s, 3H), 2.03 |
| | | | (s, 3H), 2.02 (s, 3H), |
| | | | 1.90-1.45 (m, |
| | | | 8H). |
| | | | ¹³C NMR (75 MHz, |
| | | | CDCl₃) δ 176.2, |
| | | | 171.2, 170.7, 169.5, |
| | | | 169.3, 92.7, 72.8, |
| | | | 72.6, 67.8, 61.7, 52.7, |
| | | | 45.9, 30.2 (2C), 25.77, |
| | | | 25.74, 20.8, 20.7, |
| | | | 20.63, 20.57. MALDI- |
| | | | TOF m/z Calcd for |
| | | | [C₂₀H₂₉NO₁₀ + Na]⁺ |
| | | | 466.1689; Found |
| | | | 466.1659 |
| xii | 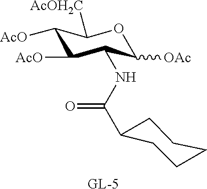 | (3R,4R,5S,6R)-6- (acetoxymethyl)-3- (cyclohexanecarboxamido) tetrahydro-2H- pyran-2,4,5-triyl triacetate/Acetyl 3,4,6-tri-O-acetyl-2- cyclohexanecarboxamido- 2-deoxy-α/β-D- gluco-hexopyranoside | Yield: 64% (760 mg) ¹H NMR (300 MHz, CDCl₃) δ 5.77 (d, J = 9.3 Hz, 1H), 5.70 (d, J = 9.0 Hz, 1H), 5.20 (dd, J = 9.6 Hz, 9.6 Hz, 1H), 5.13 (dd, J = 9.6 Hz, 9.6 Hz, 1H), 4.36 (dd, J = 9.6 Hz, 9.6 Hz, 1H), 4.26 (dd, J = 12. 6 Hz, 4.8 Hz, 1H), 4.13 |
| | | | (dd, J = 12.6 Hz, 2.1 Hz, |
| | | | 1H), 3.83 (ddd, J = |
| | | | 9.3 Hz, 4.8 Hz, 2.7 Hz, |
| | | | 1H), 2.09 (s, 3H), |
| | | | 2.08 (s, 3H), 2.05 (s, |
| | | | 3H), 2.03 (s, 3H), 1.99 |
| | | | (m, 1H) 1.83-1.60 |
| | | | (m, 4H), 1.45-1.10 |
| | | | (m, 6H). |
| | | | ¹³C NMR (75 MHz, |
| | | | CDCl₃) δ 176.1, |
| | | | 171.2, 170.7, 169.5, |
| | | | 169.3, 92.7, 73.0, |
| | | | 72.6, 67.9, 61.8, 52.4, |
| | | | 45.6, 29.4 (2C), 25.6 |
| | | | (3C), 20.8, 20.7, 20.6, |
| | | | 20.5. MALDI-TOF |
| | | | m/z Calcd for |
| | | | [C₂₁H₃₁NO₁₀ + Na]⁺ |
| | | | 480.1846; Found |
| | | | 480.1840. |
| xiii | 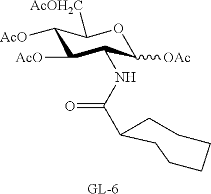 | (3R,4R,5S,6R)-6- (acetoxymethyl)-3- (cycloheptanecarboxamido) tetrahydro-2H- pyran-2,4,5-triyl triacetate/Acetyl 3,4,6-tri-O-acetyl-2- cycloheptanecarboxam ido-2-deoxy-α/β-D- gluco-hexopyranoside | Yield: 56% (680 mg) ¹H NMR (300 MHz, CDCl₃) δ 5.68 (d, J = 8.7 Hz, 1H), 5.65 (d, J = 9.0 Hz, 1H), 5.17 (dd, J = 9.6 Hz, 9.6 Hz, 1H), 5.11 (dd, J = 9.3 Hz, 9.3 Hz, 1H), 4.32 (dd, J = 9.6 Hz, 9.6 Hz, 1H), 4.25 (dd, J = 12.3 Hz, 4.8 Hz, 1H), 4.12 (dd, J = 12.3 Hz, |
| | | | 2.1 Hz, 1H), 3.81 |
| | | | (ddd, J = 9.3, 4.5, 2.1 Hz, |
| | | | 1H), 2.15 (m, 1H), |
| | | | 2.08 (s, 3H), 2.07 (s, |
| | | | 3H), 2.03 (s, 3H), 2.01 |
| | | | (s, 3H), 1.80-1.65 |
| | | | (m, 4H), 1.60-1.35 |
| | | | (m, 8H). |
| | | | ¹³C NMR (75 MHz, |
| | | | CDCl₃) δ 177.2, |
| | | | 171.2, 170.7, 169.5, |
| | | | 169.3, 92.73, 73.0, |
| | | | 72.5, 67.9, 61.8, 52.5, |
| | | | 47.6, 31.4 (2C), 28.02, |
| | | | 27.96, 26.45, 26.39, |
| | | | 20.8, 20.7, 20.6, |
| | | | 20.5. MALDI-TOF |
| | | | m/z Calcd for |
| | | | [C₂₂H₃₃NO₁₀ + Na]⁺ |
| | | | 494.2002; Found |
| | | | 494.2130 |

TABLE 2

| S. | Compound | |
|---|---|---|
| No. | Structure | Compound name |
| | Formula II (ManNAc Series) | |
| xiv | 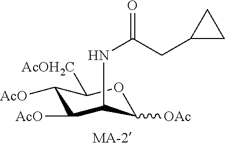 | (3S,4R,5S,6R)-6-(acetoxymethyl)-3-(2- cyclopropylacetamido)tetrahydro-2H-pyran- 2,4,5-triyl triacetate/Acetyl 3,4,6-tri-O- acetyl-2-cyclopropylacetamido-2-deoxy- α/β-D-manno-hexopyranoside |
| xv | 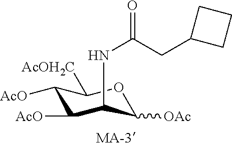 | (3S,4R,5S,6R)-6-(acetoxymethyl)-3-(2- cyclobutylacetamido)tetrahydro-2H-pyran- 2,4,5-triyl triacetate//Acetyl 3,4,6-tri-O- acetyl-2-cyclobutylacetamido-2-deoxy--α/β- D-D-manno-hexopyranoside |
| xvi | 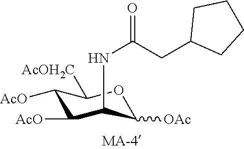 | (3S,4R,5S,6R)-6-(acetoxymethyl)-3-(2- cyclopentylacetamido)tetrahydro-2H-pyran- 2,4,5-triyl triacetate/Acetyl 3,4,6-tri-O- acetyl-2-cyclopentylacetamido-2-deoxy- α/β-D-manno-hexopyranoside ¹H NMR (300 MHz, CDCl₃) δ 6.01 (d, 1H, J = 3.0 Hz), 5.72 (d, 1H, J = 9.0 Hz), 5.32 (dd, 1H, J = 9.0 Hz), 5.18 (t, 1H, J = 9.0 Hz), 4.65 (ddd, 1H), 4.28 (dd, 1H, J = 12 Hz), 4.07-4.01 (m, 1H), 2.25 (s, 2H), 2.17 (s, 3H), 2.09 (s, |
| | | 3H), 2.05 (s, 3H), 1.99 (s, 3H), 1.88-1.84 |
| | | (broad, 1H), 1.707-1.528 (broad, 5H), 1.25- |
| | | 1.19 (broad, 4H). |
| xvii | 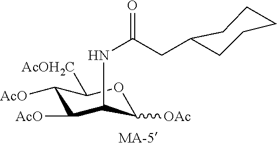 | (3S,4R,5S,6R)-6-(acetoxymethyl)-3-(2- cyclohexylacetamido)tetrahydro-2H-pyran- 2,4,5-triyl triacetate/Acetyl 3,4,6-tri-O- acetyl-2-cyclohexylacetamido-2-deoxy-α/β- D-manno-hexopyranoside |
| xviii | 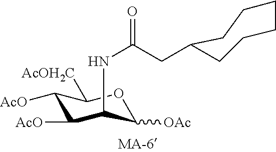 | (3S,4R,5S,6R)-6-(acetoxymethyl)-3-(2- cycloheptylacetamido)tetrahydro-2H-pyran- 2,4,5-triyl triacetate/Acetyl 3,4,6-tri-O- acetyl-2-cycloheptylacetamido-2-deoxy- α/β-D-manno-hexopyranoside |
| | Formula III (GalNAc Series) | |
| xix | 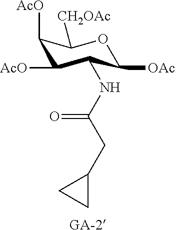 | (3R,4R,5R,6R)-6-(acetoxymethyl)-3-(2- cyclopropylacetamido)tetrahydro-2H-pyran- 2,4,5-triyl triacetate/Acetyl 3,4,6-tri-O- acetyl-2-cyclopropylacetamido-2-deoxy- α/β-D-galacto-hexopyranoside |
| xx | 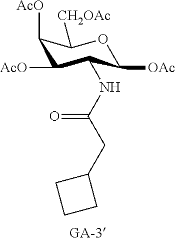 | (3R,4R,5R,6R)-6-(acetoxymethyl)-3-(2- cyclobutylacetamido)tetrahydro-2H-pyran- 2,4,5-triyl triacetate/Acetyl 3,4,6-tri-O- acetyl-2-cyclobutylacetamido-2-deoxy-α/β- D-galacto-hexopyranoside |
| xxi | 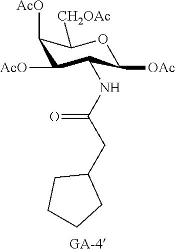 | (3R,4R,5R,6R)-6-(acetoxymethyl)-3-(2- cyclopentylacetamido)tetrahydro-2H-pyran- 2,4,5-triyl triacetate/Acetyl 3,4,6-tri-O- acetyl-2-cyclopentylacetamido-2-deoxy- α/β-D-galacto-hexopyranoside ¹H NMR (300 MHz, CDCl₃) δ 5.70 (d, 1H, J = 9.0 Hz), 5.37 (m, 1H, J = 3.0 Hz), 5.09 (dd, 1H, J = 12 Hz, 9.0 Hz), 4.47 (dt, 1H), 4.21- 4.08 (m, 3H), 4.01 (dt, 1H, J = 6.0 Hz), 2.17 (s, 2H), 2.13(s, 3H), 2.12 (s, 3H), 2.04 (s, 3H), 2.00 (s, 3H), 1.76-1.72 (broad, 1H), 1.61(s), 1.585-1.469 (m, 4H), 1.08-1.07 (m, 4H). HR-ESI-MS Calcd for C₂₁H₃₁NO₁₀ + Na⁺ 480.1840; found 480.1830 |
| xxii | 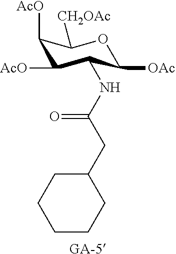 | (3R,4R,5R,6R)-6-(acetoxymethyl)-3-(2- cyclohexylacetamido)tetrahydro-2H-pyran- 2,4,5-triyl triacetate/Acetyl 3,4,6-tri-O- acetyl-2-cyclohexylacetamido-2-deoxy-α/β- D-galacto-hexopyranoside |
| xxiii | 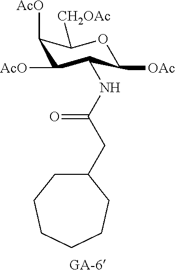 | (3R,4R,5R,6R)-6-(acetoxymethyl)-3-(2- cycloheptylacetamido)tetrahydro-2H-pyran- 2,4,5-triyl triacetate/Acetyl 3,4,6-tri-O- acetyl-2-cycloheptylacetamido-2-deoxy- α/β-D-galacto-hexopyranoside |
| | Formula IV (GlcNAc Series) | |
| xxiv | 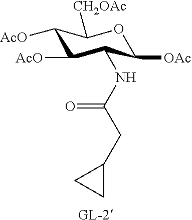 | (3R,4R,5S,6R)-6-(acetoxymethyl)-3-(2- cyclopropylacetamido)tetrahydro-2H-pyran- 2,4,5-triyl triacetate/Acetyl 3,4,6-tri-O- acetyl-2-cyclopropylacetamido-2-deoxy- α/β-D-gluco-hexopyranoside |
| xxv | 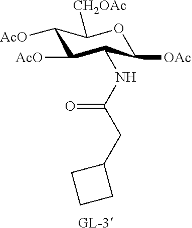 | (3R,4R,5S,6R)-6-(acetoxymethyl)-3-(2- cyclobutylacetamido)tetrahydro-2H-pyran- 2,4,5-triyl triacetate/Acetyl 3,4,6-tri-O- acetyl-2-cyclobutylacetamido-2-deoxy-α/β- D-gluco-hexopyranoside |
| xxvi | 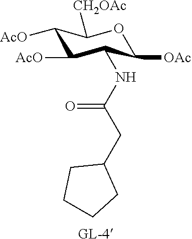 | (3R,4R,5S,6R)-6-(acetoxymethyl)-3-(2- cyclopentylacetamido)tetrahydro-2H-pyran- 2,4,5-triyl triacetate/Acetyl 3,4,6-tri-O- acetyl-2-cyclopentylacetamido-2-deoxy- α/β-D-gluco-hexopyranoside |
| xxvii | 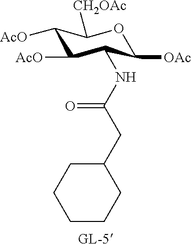 | (3R,4R,5S,6R)-6-(acetoxymethyl)-3-(2- cyclohexylacetamido)tetrahydro-2H-pyran- 2,4,5-triyl triacetate/Acetyl 3,4,6-tri-O- acetyl-2-cyclohexylacetamido-2-deoxy-α/β- D-gluco-hexopyranoside |
| xxviii | 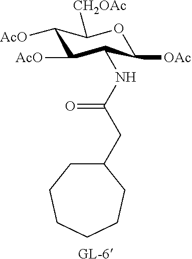 | (3R,4R,5S,6R)-6-(acetoxymethyl)-3-(2- cycloheptylacetamido)tetrahydro-2H-pyran- 2,4,5-triyl triacetate/Acetyl 3,4,6-tri-O- acetyl-2-cycloheptylacetamido-2-deoxy- α/β-D-gluco-hexopyranoside |

### Example 2

The compounds of the present invention when processed through the various glycan biosynthetic pathways in mammalian cells could result in complete or partial replacement of wild-type glycans with engineered glycans.

### Biosynthesis of Sialoglycoconjugates

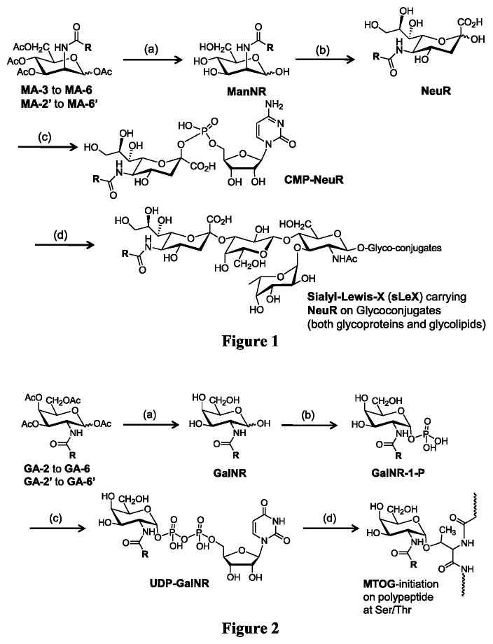

FIG. 1 depicts the compounds of present invention (Formula II) in modulating the biosynthetic pathways (i.e) in the biosynthesis of sialoglycoconjugates. Peracetylated ManNAc compounds (MA-3 to MA-6 and MA-2′ to MA-6′) (a) cross the plasma membrane through pinocytosis and get hydrolyzed to the free ManNAc analogues through the action of intracellular esterases, (b) get converted to the corresponding N-acyl-neuraminic acid (NeuR) analogues through reaction with phosphoenol pyruvate (PEP), (c) get activated to CMP-NeuR derivative and (d) utilized by various sialyl transferases for decoration on to the recipient glycoconjugates (glycoproteins, glycolipids, and glyco-RNA). Expression of modified sialoglycoconjugates has the potential to modulate cell-cell, cell-matrix, and cell-pathogen interactions as well as cell migration (extravasation, intravasation, and cancer metastasis).

### Biosynthesis of Mucin-Type O-Glycans Through the GalNAc Salvage Pathway

FIG. 2 depicts the compounds of present invention (Formula III) in modulating the biosynthesis of mucin-type O-glycans through the GalNAc salvage pathway. Peracetylated GalNAc compounds (GA-2 to GA-6 and GA-2′ to GA-6′) were (a) taken up by mammalian cell through pinocytosis and deacetylated by intracellular esterases, (b) converted to respective GalNAc-1-P derivatives through the salvage pathway, (c) activated to the UDP-GalNAc analogues, and (d) utilized by ppGalNAcTs (20 isoforms known in humans) resident in ER/Golgi to yield Tn-antigen (GalNAc-α-Ser/Thr) or its analogues. Attachment of GalNAc or its analogues serves as an initiating monosaccharide for further elaboration to core 1-core 8 glycan structures collectively known as MTOG. MTOG also carry poly(LacNAc) chains as well as A, B, and H (O) blood group antigens which participate in various glycan-protein interactions governing cellular processes.ppGal-NAcT, UDP-GalNAc:polypeptideN-acetyl-D-galactosamine transferases; LacNAc, N-acetyl-D-lactosamine; ER, endoplasmic reticulum.

### Engineering of β-O-GlcNAc-Ylation of Nuclear and Cytoplasmic Proteins

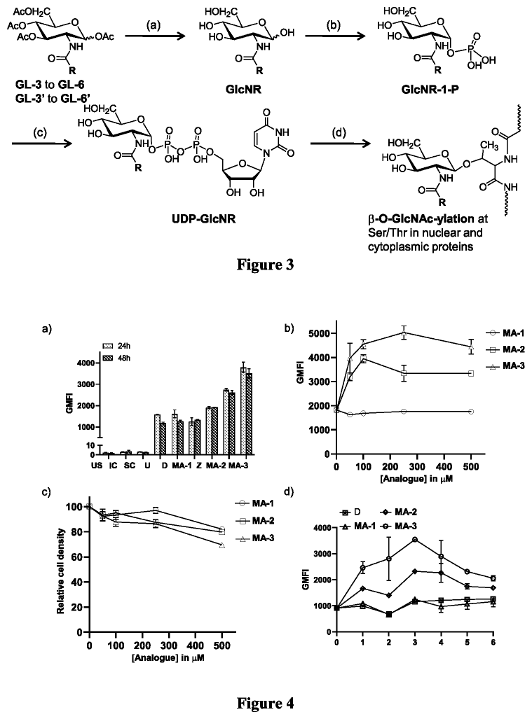

FIG. 3 illustrates the compounds of present invention (Formula IV) in engineering of β-O-GlcNAc-ylation of nuclear and cytoplasmic proteins. Peracetylated GlcNAc compounds (GL-3 to GL-6 and GL-2′ to GL-6′) are (a) taken up by mammalian cells through pinocytosis and de-acetylated by intracellular esterases, (b) converted to GlcNAc-1-P analogues, (c) activated to corresponding UDP-GlcNR analogues, and (d) utilized by β-O-GlcNAc transferase (OGT) for modification of nuclear and cytoplasmic proteins. β-O-GlcNAc-ylation of nuclear and cytoplasmic proteins, including transcription factors, competes with intracellular protein phosphorylation and finetunes the intracellular signaling processes. Note that β-O-GlcNAc-ylation could be modulated by the GalNAc analogues as well after C-4 epimerization by GALE (UDP-Gal/UDP-GalNAc C-4 epimerase) enzyme in the metabolic biosynthetic pathways.

### Example 3

### In Vitro Evaluations for Modulation of Cell Surface Glycosylation Preparation of Stock Solutions

Stock solutions of the compounds of the Formula II, III and IV prepared by the process above, were prepared in dimethylsulphoxide (DMSO) at a concentration of either 50 mM or 100 mM and sterile filtered using a PTFE 0.2 μm filter in to microcentrifuge tubes and stored at a temperature in the range of 2-4° C.

### Cell Culture Conditions for In Vitro Studies

HL-60 cells (human myeloid leukemiacells) were cultured in T75 flasks, under sterile conditions, in RPMI medium supplemented with 10% fetal bovine serum (FBS) and 1% penicillin/streptomycin, in a CO₂ incubator at 37° C. Cells were counted using a Beckman-Coulter Z2-cell counter. Cells growing in log phase were used for incubation with the analogues.

Measurement of Cell Surface CD15s (sLeX) Levels by Flow Cytometry

HL-60 cells (seeding density of 3.0×10⁵ cells/mL in 5.0 mL of complete medium in 60 mm diameter tissue culture dishes) were either left untreated (U) or treated separately with vehicle (DMSO, D), MA-2, and MA-3 (0-500 μM). After 24 h, cells were harvested and washed thrice with PBS. Prior to the last wash the cells were counted in Z2 Coulter counter and aliquoted in to 5×10⁵ cells per sample. Cells were stained with 5.0 μg/mL of mouse anti-human CD15s (CSLEX) in 50 μL PBS for 30 min at 4° C. The cells were washed with PBS (2×0.5 mL) and stained with AlexaFluor488-conjugated anti-mouse IgM (10 μg/mL) in 50 μL of PBS for 60 min at 4° C. Then, the cells were washed with PBS (2×0.5 mL), resuspended in 200 μL of PBS. Propidium iodide (PI) was added (2.5 μg/mL) to all the samples except controls. Two biological replicates were prepared for each condition, each sample was counted twice in BD FACSVerse for 10,000 PI-negative events, and analyzed by FlowJo v10. The error bars shown are standard deviations of at least duplicate samples; three independent experiments were performed for each study. FIG. 4a depicts the sLeX levels upon incubation with control conditions (US, unstained; IC, isotype control; SC, secondary only control; U, untreated; D, DMSO (vehicle) treated); Ac₄ManNAc (MA-1), Ac₄ManNAz (Z), Ac₄ManNCp (MA-2), and Ac₄ManNCb (MA-3) at 50 μM and measured after 24 and 48 h. FIG. 4b depicts the sLeX levels upon incubation with D, MA-1, MA-2, or MA-3 at various concentrations (0-500 μM) for 24 h. FIG. 4c depicts the relative densities of HL-60 cells incubated with analogues MA-1, MA-2, and MA-3 for 24 h as a measure of cytotoxicity. Control, cells treated with the vehicle volume corresponding to the analogue stockvolume, is taken as 100%. FIG. 4d depicts the time course of sLeX expression upon incubation with vehicle, MA-1, MA-2, or MA-3 at 50 μM measured every day for six days. MA-1 and MA-2 compounds were used for the comparative study and the structures of these compounds are provided as below.

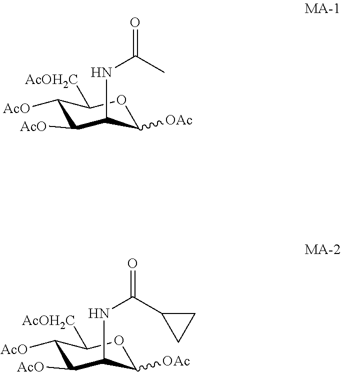

### Example 4

### Metabolic Engineering of Sialic Acid

FIG. 5 illustrates competitive processing by compounds of the present invention (ManNAc compounds). ManNAc compounds competitively reduce the expression of N-azidoacetyl-D-neuraminic acid (NeuAz)-carrying sialoglycans through the metabolic processing of Ac₄ManNAz. Jurkat (human T-lymphoma) cells were incubated with Ac₄ManNAz (Z) (50 μM) alone, co-incubated with GL-2, GL-3, MA-2, or MA-3 (at various concentrations such as 10,

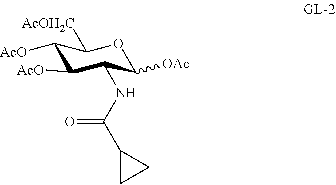

25, and 50 μM) for 24 h. Expression of N-azidoacetyl-D-neuraminic acid (NeuAz) on the cell surface sialoglycans was estimated using strain-promoted azide-alkyne cycloaddition (SPAAC) with DBCO-Cy5 and analyzed by flow cytometry. Results show that NeuAz expression was reduced competitively in a dose-dependent manner by MA-2 and MA-3 and not by GL-2 and GL-3. GL-2 was used for comparative study and the structure of this compound is as below.

### Example 5

### Experiments in Cell Cultures In Vitro

Jurkat cells (human T cell leukemia) were used as-obtained and HL-60 cells (human acute myeloid leukemia) were purchased from European collection of authenticated cell cultures (ECACC, UK). Cells were cultured in flasks or petri dishes, under sterile conditions, in RPMI 1640 supplemented with 10% fetal bovine serum (FBS) and 1% P/S (50 U/mL of penicillin and 0.05 mg/mL of streptomycin) at 37° C. in a humidified incubator maintaining 5% carbon dioxide. Cell culture work was performed inside a Baker SterilGard biosafety cabinet. Cells were counted using a Beckman Coulter Z2 particle counter in 0.45 mm filtered phosphate buffered saline (PBS) or haemocytometer.

Stock solutions of the compounds 1a-1g, 2a, 2b, and 3 (50 mM) were prepared in DMSO (D) and were filtered through 0.22 mm PTFE syringe filter. Stock solutions were added directly to cell culture keeping the total volume of DMSO to a maximum of 0.25% v/v. All the experiments were initiated using actively dividing log phase cells. Cells were seeded at a density of 3.0×10⁵ cells/mL for all the experiments, unless mentioned otherwise. Dibenzocyclooctyne-Cy5 (DBCO-Cy5; stock solution of 10 mM in DMSO) was employed for estimation of NeuAz expression using strain promoted azide-alkyne cycloaddition (SPAAC). Propidium iodide (PI) (stock solution of 1.0 mg/mL in water) was used to exclude non-viable cells. Jurkat cells (untreated, U), without treatment of vehicle, were employed as additional control in all the experiments. Cell counts were taken directly from the media, after gentle pipetting to single cell suspension, at the time of harvest in order to obtain a measure of cytotoxicity due to the analogues. FACS staining buffer (FSB), consisting of 1.0% BSA w/v and 0.05% sodium azide w/v in PBS, was used for flow cytometry experiments.

### Example 5.1

### Estimation of NeuAz Expression by Flow Cytometry

Jurkat cells (3.0×10⁵ cells/mL; 2.5 mL per well in 6-well plates) in complete medium were treated with either vehicle (D) or Compound 1c; Untreated Jurkat cells, without addition of vehicle, were also included as additional controls. Cells were harvested at 48 h, washed thrice with PBS (3×0.5 mL) (3000×g, 2.0 min). Prior to the last wash cells were counted and were aliquoted to 1.0×10⁶ cells per sample in 1.5 mL microcentrifuge tubes and centrifuged. The cells were gently resuspended in PBS (100 mL) and treated with DBCO-Cy5 (5.0 mM) and incubated at 37° C. (on a Torrey-Pines dry heating bath with mild shaking). After one hour, the cells were washed using FSB (0.5 mL, 3000×g, 2.0 min, room temperature), resuspended in PBS (250 mL), treated with PI (2.5 mg/mL), and analyzed using BD FACSCanto and FACSDiva Version 6.1.3. The cells were gated for PI-negative population for 10,000 events. Two replicate samples were prepared for each conditions and each sample was counted twice.

For the optimization of DBCO-Cy5 concentration for SPAAC, Jurkat cells cultured with D or 1c (50 mM) for 24 h were harvested, washed, and aliquoted in to 5.0×10⁵ cells per sample in PBS (100 mL). The cells were treated with DBCO-Cy5 at 0, 5, 10, 20, and 40 mM concentrations at 37° C. After 1.0 h, cells were washed in FSB (3×0.5 mL at 3000×g for 2.0 min), resuspended in PBS (250 mL), treated with PI (2.5 mg/mL), and analyzed by BD FACSVerse and FlowJo V10. The cells were gated for PI-negative population for 10,000 events. Two replicate samples were prepared for each condition and each sample was counted twice.

For the optimization of incubation time for SPAAC using DBCO-Cy5, Jurkat cells (3.0×10⁵ cells/mL in 5.0 mL cultured in 60 mm diameter petri dishes) incubated with either D or 1c (50 mM) for 24 h were harvested and washed with PBS (3×0.5 mL at 3000×g for 2.0 min). Cells were aliquoted at 5.0×10⁵ cells per sample and treated with DBCO-Cy5 (20 mM) at 37° C. Reactions were stopped at various time points (0-120 min) by washing with FSB (3×0.5 mL), resuspended in PBS (250 mL), and kept on ice-bath. Cells were treated with PI and analyzed by BD FACSVerse and FlowJo V10 as mentioned above.

Estimation of NeuAz expression by 1c in the presence of peracetylated ManNAc analogues: Jurkat cells (3.0×10⁵ cells/mL in 3.0 mL per well in 6-well plates) were incubated with (i) D, (ii) 1a, 1b, 1c, or 1d alone (50 mM) or (iii) 1a, 1b, or 1d (50 mM) in combination with 1c (50 mM). After 24 h, cells were harvested, washed using FSB, aliquoted to 5.0×10⁵ cells per sample, and processed as mentioned above for estimation of NeuAz expression using DBCO-Cy5 (20 mM). Experiments using HL-60 cells were performed using the same procedure.

Estimation of NeuAz expression upon simultaneous and delayed addition of 1c: In one condition, Jurkat cells were incubated with D, 1a, or 1b (50 mM) along with 1c (50 mM). In another condition, Jurkat cells were first incubated with D, 1a, or 1b (50 mM) alone followed by the addition of 1c (50 mM) at 12 h. After a total of 36 h, cells were harvested, washed with PBS (3×0.5 mL), aliquoted to 5.0×10⁵ cells per sample in 250 mL, reacted with DBCO-Cy5 (20 mM), washed, treated with PI, and analyzed by flow cytometry as mentioned above.

Effect of GlcNAc and ManNAc analogues on NeuAz expression: Jurkat cells were treated with 1a, 1b, 2a, or 2b (10, 25, and 50 mM for each analogue) in combination with 1c (50 mM). Cells treated with 1c (50 mM) alone were considered as a reference. After 24 h, cells were harvested, processed, labelled with DBCO-Cy5 (20 mM), and analysed by flow cytometry as mentioned above.

Effect of ManNAc analogues on NeuAz and GalNAz expression: Jurkat cells were incubated under various conditions, namely, (i) D, 1c, 3, 1a, or 1b alone, (ii) 1a or 1b in combination with 1c, (iii) 1a or 1b in combination with 3. After 24 h, cells were harvested, washed, labelled using DBCO-Cy5 (20 mM), processed, and analysed by flow cytometry as mentioned above

### Example 5.2: Estimation of Sialoglycan Epitopes on the Cell Surface by Flow Cytometry

Effect of ManNAc analogues on the expression of sialyl-Lewis-X and Lewis-X: HL-60 cells (3.0×105 cells/mL in 2.0 mL complete medium in 6 well plate) were treated with D (vehicle), 1a, 1b, 1c, 1d, 1e, 1f, or 1g (50 PM), along with untreated (U) control. After 24 h, cells were harvested and washed with PBS (3×0.5 mL, 3000×g, 3.0 min at room temperature). Prior to last wash, cells were counted using Z2 coulter counter and aliquoted at 2.5×10⁵ cells per sample and stained with either mouse anti-human CD15s (CSLEX1) (1.25 μg/mL) or mouse anti-human CD15 (HI98) (2.5 μg/mL) in HEPES/CaCl₂) buffer (30 mM HEPES, 110 mM NaCl, 10 mM KCl, 2 mM MgCl₂, 10 mM glucose, 1.5 mM CaCl₂) containing 0.1% bovine serum albumin, pH 7.3) (total volume of 100 μL) for 30 min at 4° C. Cells were washed with HEPES/CaCl₂ buffer (2×0.5 mL) and stained with AlexaFluor488-conjugated anti-mouse IgM (2.5 μg/mL) (for both CD15s and CD15) in 100 μL HEPES/CaCl₂ for 30 min at 4° C. Cells were washed with HEPES/CaCl₂ buffer (2×0.5 mL), resuspended in PBS (0.2 mL), treated with PI (2.5 μg/ml) and analysed by BD FACSVerse. Samples treated with (i) isotype antibodies followed by secondary antibody and (ii) secondary antibody alone were prepared as controls. Single Cells were gated for PI-negative population for 10,000 events and analysed by flow cytometry. Two replicate samples were prepared for each condition and each sample was counted twice. Three independent experiments were performed. Data was analysed using FlowJo V10. For plotting graph and calculation of significant probability ‘p’ values, two-way ANOVA with Dunnett's multiple comparison tests was employed in GraphPad Prism 8.

Time course of CD15s expression: HL-60 cells (3.0×10⁵ cells/mL in 2.5 mL complete medium in 6-well plates) were incubated with D, 1a, 1b, or 1d (50 mM). At each time point, from day 1 to day 6, the cells were harvested from the entire contents of the well, washed, and aliquoted to 5.0×10⁵ cells per sample, and immuno-stained using anti-CD15s (CSLEX1) antibody as mentioned above. On day 3, 4, and 5 complete media (1.0 mL) was added to each remaining well in order to compensate for media exhaustion. No contents were removed from the well during the period.

Comparison of mouse and human E-selectin binding: HL-60 cells were harvested from actively growing cultures and washed with PBS (3×0.5 mL). Prior to the last wash, cells were counted and aliquoted in to 5.0×10⁵ cells per sample. Cells were incubated with either human E-selectin-Fc chimera protein (hCD62E-Fc; 2.5 mg/mL) or mouse E-selectin-Fc chimera protein (mCD62E-Fc; 2.5 mg/mL) in PBS (50 mL) for 45 min at 4° C. Cells were then washed with PBS (2×0.5 mL), stained with PE-conjugated F(ab′)2 goat anti-human Fc (2.5 mg/mL) in PBS (50 mL) for 45 min at 4° C. Cells were then washed with PBS (2×0.5 mL), resuspended in 0.85% saline (0.2 mL) containing Sytox Red (dilution of 1:1000; stock solution of 5.0 mM in DMSO), and analyzed by flow cytometry using BD FACSVerse. Cells were gated on Sytox Red negative population and 10,000 events were counted within the gate. Two replicate samples were analyzed for each condition and each sample was counted twice. Data were analyzed using FlowJo V10.

Optimization of concentration of mouse E-selectin-Fc chimera protein: HL-60 cells were harvested from actively growing cultures and washed with PBS (3×0.5 mL). Cells were counted, aliquoted in to 5×10⁵ cells per sample, treated with mouse E-selectin-Fc chimera protein (mCD62E-Fc) at 0, 2.5, 5.0, 10, and 20 mg/mL concentrations in PBS (50 mL) and incubated at 4° C. After 1 h, cells were washed with PBS (2×0.5 mL) and stained with PE-conjugated F(ab′)2 goat anti-human Fc in PBS (50 mL) at 4° C. After 1 h, cells were washed with PBS (2×0.5 mL) and resuspended in 0.85% saline (0.2 mL) containing Sytox Red (dilution 1:1000; stock solution of 5.0 mM in DMSO). Two replicate samples were analyzed for each condition and each sample was counted twice by flow cytometry as mentioned above.

Effect of HexNAc analogues on the expression of E-selectin ligands: HL-60 cells (3.0×10⁵ cells/mL in 2.5 mL complete medium in 6-well plates) were incubated with D, 1a, 1b, or 1d (50 mM), along with untreated controls. After 72 h, cells were harvested and washed with PBS (3×0.5 mL). Prior to the last wash, cells were counted and aliquoted into 5.0×10⁵ cells per sample. Cells were stained with mCD62E-Fc chimera protein (10 mg/mL) in PBS (50 mL) at 4° C. After 1.0 h, cells were washed with PBS (2×0.5 mL) and stained with PEconjugated F(ab′)2 goat anti-human Fc in PBS (50 mL) at 4° C. After 1.0 h, cells were washed with PBS (2×0.5 mL) and resuspended in 0.85% saline (0.2 mL) containing Sytox Red (dilution 1:1000; stock concentration of 5.0 mM). Additionally, control samples of cells treated with only the secondary antibody were prepared. Two replicate samples were analyzed for each condition and each sample was counted twice by flow cytometry as mentioned above.

Effect of HexNAc analogues on the expression of cutaneous lymphocyte antigen (CLA/HECA452) epitopes: HL-60 cells (3.0×10⁵ cells/mL in 2.5 mL complete medium in 6-well plates) were treated with D, 1a, 1b, or 1d (50 mM), including untreated controls. After 24 h cells were harvested, washed in PBS (3×0.5 mL), aliquoted to 5.0×10⁵ cells per sample, and incubated with rat anti-human CLA (HECA452) antibody (5.0 mg/mL) in PBS (50 mL) at 4° C. After 1.0 h, cells were washed with PBS (2×0.5 mL) and stained with AlexaFluor488-conjugated anti-rat IgM (10 mg/mL) in PBS (50 mL) at 4° C. After 1.0 h, cells were washed with PBS (2×0.5 mL), resuspended in PBS (0.2 mL) and treated with PI (2.5 mg/mL). Additionally control samples of cells treated with (i) isotype antibody followed by secondary antibody and (ii) only secondary antibody were prepared. Cells were gated on PI-negative population and analyzed by flow cytometry as given above. Two replicate samples were analyzed for each condition and each sample was counted twice by flow cytometry as mentioned above.

### Example 5.3 Western and Avidin (Far-Western) Blotting Experiments

Effect of ManNAc analogues on expression of CD15s epitopes: HL-60 cells (3.0×10⁵ cells/mL in 10 mL complete medium in 10 cm diameter petri dishes) were incubated with D, 1a, 1b, 1d, 1e, 1f, or 1g (50 mM), along with untreated (U) controls. After 72 h, cells were harvested, washed with PBS (3×5.0 mL), and lysed in RIPA buffer (10⁷ cells in 100 mL), containing protease inhibitor cocktail (PIC, 1:100). Protein concentration of the soluble fraction was estimated using the Bradford assay. Proteins from total lysates were resolved by 7.5% SDS-PAGE and blotted onto nitrocellulose membrane (constant current of 250 mA for 3.0 h at 4° C.). Membranes were blocked using non-fat milk (NFM) (5.0% w/v) in PBS and incubated with primary antibodies (5.0 mL PBS containing 5.0% NFM) overnight at 4° C. Membranes were washed with 0.1% PBS-T (0.1% v/v tween-20 in PBS; 3×5.0 mL, five min each at room temperature) followed by incubation with appropriate fluorophore or horse radish peroxidase (HRP) conjugated secondary antibodies for 1.0 h at room temperature. Membranes were washed with 0.1% PBS-T (3×5.0 mL, five min each) and then either directly scanned using Amersham Typhoon fluorescence scanner or developed on photographic films after treatment with enhanced chemiluminescence reagents. At least two independent replicate experiments were performed for all the blots.

The primary and secondary antibodies employed, along with dilution and stock concentration, are given below: Primary antibodies: Anti-CD15s (CSLEX1; IgM, dilution 1:5000; stock concentration 0.5 mg/mL) Anti-CLA (HECA452; IgM, dilution 1:1000, stock concentration 0.5 mg/mL) Anti-3-actin (AC15, IgG, dilution 1:20000, stock concentration 2.2 mg/mL); and Secondary Antibodies: AlexaFluor488-conjugated anti-mouse IgM (1:10000, stock concentration 2.0 mg/mL) AlexaFluor488-conjugated anti-rat IgM (1:10000, stock concentration 0.5 mg/mL) HRP-conjugated anti-mouse IgG (1:10000, stock concentration 0.8 mg/mL) Cy5-conjugated donkey anti-mouse IgG (1:10000, stock concentration 1.4 mg/mL) AlexaFluorplus488-conjugated anti-mouse IgG (1:10000, stock conc. 2.0 mg/mL).

Effect of ManNAc analogues on expression of MAL-II and SNA epitopes: HL-60 cells (3.0×10⁵ cells/mL in 10 mL complete medium in 10 cm diameter Petri dishes) were incubated with D, 1a, 1b, or 1d (50 mM), along with untreated (U) controls. After 72 h, cells were harvested and lyzed in RIPA buffer containing PIC. Protein concentration in the soluble fraction was estimated using Bradford assay, subjected to 10% SDS-PAGE, blotted onto nitrocellulose membranes (constant current 250 mA, 3 h, 4° C.). Membranes were blocked using 2% w/v gelatin (5.0 mL) in 0.1% PBS-T at room temperature. After 1h, membranes were incubated with either bMAL-II (1:2000, stock conc. 1.0 mg/mL) or bSNA (1:5000, stock conc. 2.0 mg/mL) in 0.1% PBS-T (5.0 mL) at room temperature. After 1 h, membranes were washed using 0.1% PBS-T (6×5.0 mL, 5.0 min), followed by incubation with HRPconjugated avidin (1:50000, stock conc. 2.0 mg/mL) in 0.1% PBS-T (5.0 mL). After 30 min, membranes were washed using 0.1% PBS-T (6×5.0 mL, 5.0 min). Blots were developed using enhanced chemiluminescence substrate and photographic films. Blots using anti-β-actin (as mentioned above) were performed in parallel as loading controls. At least two independent replicate experiments were performed.

Effect of ManNAc analogues on cell adhesion to E-selectin and L-selectin coated surfaces: This assay was performed in 96-well polystyrene plates under sterile conditions. The plates were prepared freshly on the day of adhesion experiments. Wells were first coated with protein-G (200 ng in 50 μL PBS per well) for 1.0 h at room temperature (RT) followed by PBS washes (2×0.1 mL). Then either recombinant mouse E-selectin-Fc chimera or L-selectin-Fc chimera (20 ng in 50 μL PBS per well) was added to the wells and plate was left undisturbed for 1.0 h at RT. Wells were aspirated and washed with PBS (2×0.1 mL) followed by blocking with 1.0% (w/v) BSA in 50 μL PBS per well for 1.0 h at RT. After blocking, the wells were washed with PBS (3×0.1 mL).

HL-60 cells (3.0×10⁵ cells per mL; 2.0 mL in six well plates) were incubated with 50 μM ManNAc analogues, harvested at 48 h, washed once with serum free RPMI medium (0.5 mL), and resuspended in serum free RPMI 1640 media containing 1.5 mM CaCl₂) (4.0×10⁵ cells/mL in 1.0 mL). The single cell suspension was added to each well (2.0×10⁴ cells in 50 μL media per well) and allowed to adhere for 1.0 h at 37° C. in a CO₂ incubator. Thereafter, the wells were washed with PBS (2×0.1 mL), cells were fixed with 4.0% (w/v) paraformaldehyde (PFA) in PBS (50 mL) for 10 min followed by staining with DAPI (1:3000) in 50 μL PBS for 20 min after PBS washes (3×0.1 mL). Excess stain was removed with PBS (3×0.1 mL) and 50 μL PBS was added to each well. Wells coated and blocked with protein-G and BSA, respectively, (without selectins) served as negative controls. Images were captured at 10× magnification using Olympus IX53 fluorescence microscope. At least two fields were acquired from each well. For each condition, there were three replicate wells. The images were analyzed manually and the total number of adherent cells per well for each condition were plotted.

### Example 5.4 Estimation of Total Sialic Acids by Periodate-Resorcinol Assay

Total sialic acids were measured following reported procedures. HL-60 cells (3.0×10⁵ cells/mL in 10 mL complete medium in 10 cm diameter Petri dishes) were incubated with D, 1a, 1b, 1c, 1d, 1e, 1f, 1g, 2a, or 2b (100 mM each), along with untreated (U) controls. After 48 h, cells were harvested, washed with PBS (3×5.0 mL; 3000×g, for 2.0 min), aliquoted to 2×10⁶ cells per sample and resuspended in PBS (150 mL) in 1.5 mL microcentrifuge tubes. Cells were lyzed by three freeze-thaw cycles and the total lysates were subjected to periodate-resorcinol assay. Briefly, lysates (150 mL) were treated with 0.4 M aqueous periodic acid (6.67 mM) or (2.5 mL) and incubated either on ice for 15 min (for measurement of total sialic acids) or at 37° C. for 1.0 h (for measurement of glycoconjugate bound sialic acids). About 10 mL of reagent cocktail was prepared using 6% w/v of aqueous resorcinol (1.0 mL), 2.5 mM copper (II) sulfate (1.0 mL), 44% of concentrated hydrochloric acid (4.4 mL), and water (3.6 mL). The mixture was then treated with the reagent cocktail (250 mL per sample), boiled at 100° C. for 10 min, rapidly quenched in an ice-bath and treated with tert-butanol (250 mL). The contents were centrifuged and the supernatants were aliquoted (200 mL) into 96-well plates and the absorbance at 630 nm was measured. At least three replicates were prepared for each condition.

### Example 6

Endogenous sialic acid biosynthesis begins from the conversion of UDP-GlcNAc to ManNAc by the enzyme bifunctional UDP-N-acetylglucosamine 2-epimerase/N-acetylmannosamine kinase (GNE/MNK) followed by aldol reaction with phosphoenol pyruvate to yield N-acetyl-D-neuraminic acid (NeuAc). NeuAc is further converted to the activated sugar nucleotide donor in the form of CMP-NeuAc, which is utilized by sialyl transferases stationed in the Golgi apparatus as shown in FIG. 1. Intracellular concentrations of sialic acids are maintained at homeostatic levels by feedback inhibition of GNE/MNK by CMP-NeuAc. However, exogenous supply of ManNAc or its analogues could bypass this feedback inhibition and increase the flux of biosynthesis of NeuAc or the corresponding analogues. Depending on the chemical structure and their steric hindrance, ManNAc analogues are processed to varying degrees of incorporation by the enzymes of the sialic acid pathway.

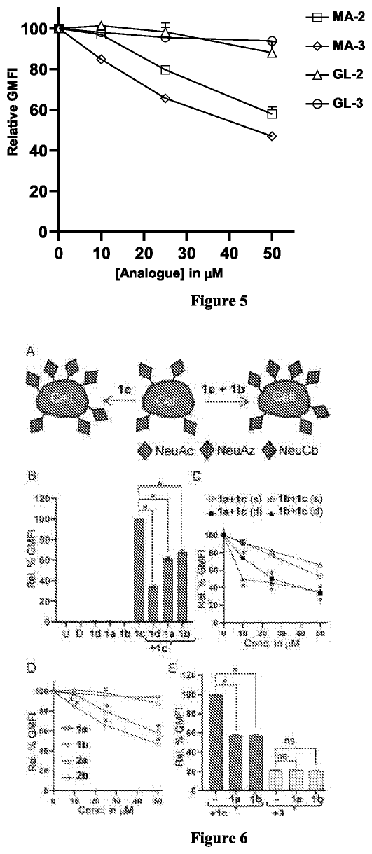

ManNAc analogues carrying N-(cycloalkyl)acyl moieties, Compounds 1a and 1b, 1f converted to sialic acids, are not amenable to direct estimation in contrast to those carrying bio-orthogonally reactive functional groups. Therefore an indirect approach was resorted wherein cells were treated with Ac₄ManNAz (1c) in the presence or absence of 1a or 1b. The control (untreated and vehicle treated) cells would express the wild-type NeuAc and the cells treated with 1c would express both NeuAc and N-azidoacetyl-D-neuraminic acid (NeuAz); whereas the cells treated with either 1a or 1b, in the presence of 1c, 1f amenable to metabolism, would result in reduced expression of NeuAz along with N-cyclopropanoyl-D-neuraminic acid (NeuCp) or N-cyclobutanoyl-D-neuraminic acid (NeuCb), respectively, in a competitive manner (FIG. 6A).

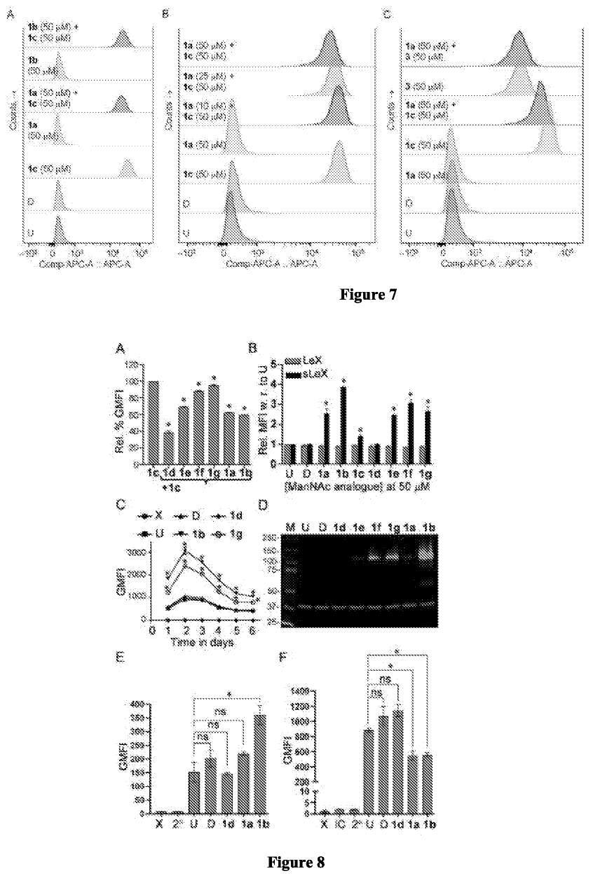

In order to evaluate the metabolism, Jurkat (human T-lymphoma) cells were selected and incubated with ManNAc analogues either alone (50 μM) or in combination with 1c (50 μM) for 48 h. Expression of NeuAz on cell surface was estimated by flow cytometry through the copper-free bio-orthogonal strain-promoted azide-alkyne cycloaddition (SPAAC) reaction using dibenzocyclooctyne-Cy5 (DBCO-Cy5). The conditions for click reaction were optimized for various concentrations of DBCO-Cy5 and time course of SPAAC reaction in Jurkat cells. Based on the optimization results, incubation time of 30 min at 37° C. for 5.0×10⁵ cells (pre-incubated with 1c for 48 h) with 20 μM of DBCO-Cy5 in 100 μL total volume for experiments with ManNAc analogues was chosen. As expected, cells treated with compound 1c alone showed robust expression of NeuAz (taken as 100%), but the NeuAz expression was reduced to 30%, 60% and 65% upon co-incubation with 1d, 1a, and 1b respectively. Control conditions of untreated cells and cells treated with vehicle (DMSO, D), 1a, 1b, or 1d alone showed negligible signals upon reaction with DBCO-Cy5 (FIG. 6B and FIG. 7A). These results indicated that the compound 1d was able to compete and interrupt metabolism of 1c to a maximum extent with a 70% reduction while 1a and 1b effected ˜35-40% reduction. In order to test the dose-dependency of this inhibitory effect on NeuAz expression and to establish competitive metabolic processing, Jurkat cells were first treated with either 1a or 1b (0-50 μM) followed by addition of 1c (50 μM) at 12 h and estimation of NeuAz at 48 h. As expected, the suppression of NeuAz expression was substantially higher when added with a 12 h delay compared to simultaneous addition, in a dose-dependent manner. At 10 μM, delayed addition resulted in 30% and 50% suppression upon treatment with 1a and 1b, respectively. By contrast simultaneous addition resulted only in a 10% reduction of NeuAz for both 1a and 1b (FIG. 6C and FIG. 7B). These results suggested that both 1a and 1b are amenable to sialic acid biosynthesis and that 1b showed better competitive ability compared to 1a.

It was observed that the reduction of NeuAz expression is specific to ManNAc analogues. Jurkat cells were incubated with 1a, 1b, 2a, or 2b (0-50 μM) in 5 combination with 1c (50 μM; simultaneous addition) for 24 h. At 50 μM, NeuAz expression was suppressed, in a dose-dependent manner, to 60% and 50% of controls upon treatment, respectively, with 1a and 1b. In contrast, only negligible reduction in NeuAz expression was observed upon co-incubation with the C-2 GlcNAc epimers 2a or 2b (FIG. 6D). These results confirmed the specificity of ManNAc analogues for processing through the sialic acid pathway and the inability of GlcNAc analogues to interfere in the biosynthesis of NeuAz from 1c.

The high degree of selectivity of ManNAc analogues for processing by the sialic acid biosynthetic pathway was further confirmed by competitive studies using Ac₄GalNAz (3) which is known to be processed through both the mucin-type O-glycosylation (MTOG) and β-O-GlcNAc-ylation pathways. The expression of NeuAz by 1c was suppressed by 1a or 1b (50 μM) to an extent of 50% upon co-incubation. By contrast, no reduction in the expression of N-azidoacetyl-D-galactosamine (GalNAz) on the cell surface was noticed upon co-incubation of 3 (50 μM) with 1a or 1b (50 μM) (FIG. 6E and FIG. 7C). These results confirmed that the ManNAc analogues did not interfere or suppress GalNAz processing through MTOG and β-O-GlcNAc.

Having confirmed metabolic acceptability in Jurkat cells, the compounds were tested on HL-60 (human acute myeloid leukemia) cells which are a reliable model for neutrophil adhesion. The effect on competitive suppression of NeuAz expression was studied using 1c as a reporter. HL-60 cells were treated with 1c either alone (50 μM) or in combination with ManNAc analogues (50 μM), including the straight chain derivatives, for 24 h followed by DBCO-Cy5 ligation. Flow cytometry analysis showed that 1a, 1b, 1d, 1e, 1f, and 1g reduced the NeuAz expression, respectively, to 60%, 55%, 40%, 65%, 85%, and 90% with respect to 1c alone (taken as 100% for control) (FIG. 8A). Maximum suppression was observed for the N-acetyl (wild-type) analogue 1d, followed by the cycloalkyl derivatives 1a and 1b; the straight chain analogues 1e showed significant suppression, 1f showed moderate suppression, and 1g showed mild effect on NeuAz expression. The decreasing ability of linear alkyl moieties (1d-1g), as a function of chain length, for suppression of NeuAz expression is in contrast to the cycloalkyl moieties. While 1b induced a reduction of 45%, the corresponding straight chain analogue with five carbons 1g showed only a mild reduction of 10% with respect to controls. Similarly, 1a induced a reduction of 40% compared to only a 15% reduction by 1f. These results demonstrated that the cycloalkyl groups at the N-acyl chain of ManNAc are significantly more acceptable for metabolic processing compared to their straight chain analogues.

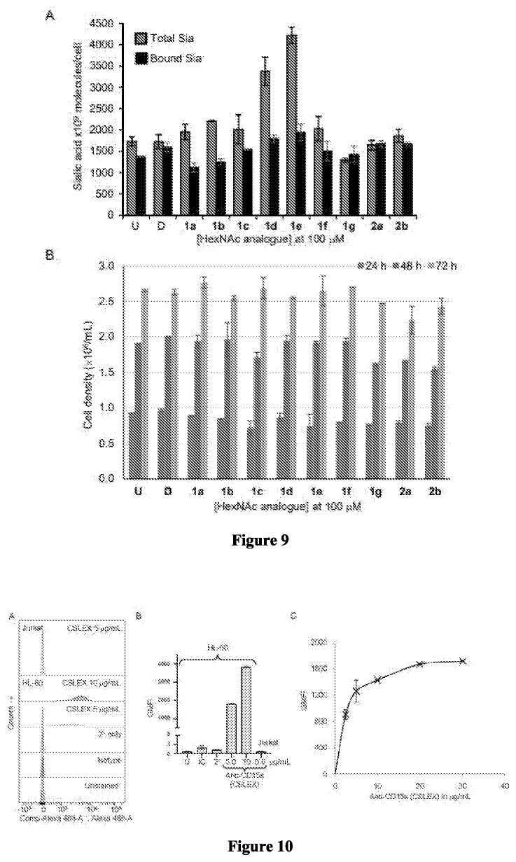

To verify metabolic processing to sialic acids, both total and glycoside bound sialic acid levels were measured using the periodate-resorcinol assay in HL-60 cells (FIG. 9). Total sialic acid levels were comparable to untreated cells for treatment with vehicle (D), the GlcNAc analogues 2a, or 2b (100 μM, 48 h); an increase of 1.14 to 1.3-fold was observed for treatment with 1a, 1b, 1c, and 1f. An increase of 2.0-fold and 2.3-fold was observed for 1d and 1e, while 1g showed a decrease by 0.7-fold. The glycoside bound sialic acid levels were comparable to untreated cells for D, 1c, 1f, 2a, and 2b. About ˜10% increase was observed for both 1d and 1e while both 1a and 1b showed a decrease of ˜10%. The ratios of total sialic acids to glycoside bound sialic acids were similar to untreated cells for D, 1c, and 1f; higher ratios of free sialic acid were observed for 1a, 1b, 1d, and 1e while lower ratios were observed for 1g, 2a, and 2b. It was found that treatment with 1d and 1e resulted in large increase in the metabolic flux of free sialic acid with modest gains in glycoside-bound sialic acids; whereas treatment with 1a and 1b resulted in a gain in free sialic acids with a moderate decrease in glycoside bound sialic acids. It can be seen that the compound 1a increased the total sialic acid levels by two folds in HEK293 cells with more than 50% being the modified N-cyclopropanoyl-D-neuraminic acid. In the case of treatment with 1g, 2a, and 2b almost all the sialic acids were glycoside bound.

Comparative analysis revealed that the compound 1b resulted in increased flux of sialic acid biosynthesis whereas the corresponding five carbon straight chain N-pentanoyl analogue 1g did not show any effect, suggesting that steric effects played a prominent role; whereas both the N-cyclopropanoyl analogue 1a and the corresponding straight chain N-butanoyl analogue 1f showed similar total sialic acid levels. Cell density measured at 24, 48, and 72 h, as a measure of proliferation, upon incubation with 1a-1g, 2a, and 2b (100 μM, 48 h) exhibited negligible toxicity in all cases, with respect to controls, with the exception of 1c, 1g, 2a, and 2b which exhibited a mild decrease of ˜10% in cell density (FIG. 9).

With the ability to modulate the structure of sialic acids on the cell surface using N-(cycloalkyl)acyl analogues were ventured to investigate changes to established sialic-acid carrying epitopes of immunological importance, viz., sialyl-Lewis-X (sLeX, CD15s) using anti-CD15s (CSLEX1) antibody. Optimal conditions for flow cytometry staining for cells, including isotype and secondary antibody only controls, were obtained by titration at various concentrations of anti-CD15s for saturating concentrations. Consistent with the literature, HL-60 cells were positive and Jurkat cells were negative for CSLEX1 epitopes (FIG. 10). Interestingly, HL-60 cells upon incubation with 1b (50 μM, 24 h) showed nearly a four-fold increase in the binding of CSLEX1 antibody, compared to controls (untreated, vehicle-treated, and 1d) (FIG. 8B). Treatment with 1f resulted in a three-fold increase; the three analogues, viz., 1a, 1e, and 1g resulted in an increase of ˜2.5-fold while 1c showed a moderate increase of 1.4-fold. Notably, N-butanoyl analogue 1f showed higher CSLEX1 binding compared to the four-carbon N-cyclopropanoyl analogue 1a; whereas the N-pentanoyl analogue 1g showed lower CSLEX1 binding compared to the five-carbon N-cyclobutanoyl analogue 1b. These results suggest unique structure-based differential effects of the N-acyl moiety for metabolic processing and sLeX display.

Increase in sLeX/CD15s levels due to the utilization of its precursor Lewis-X (LeX/CD15) was tested. Measurement of cell surface CD15 levels using anti-CD15 (HI98) in HL-60 cells treated with ManNAc analogues (1a-1g, 50 μM for 24 h) by flow cytometry revealed no major changes, compared to controls, under all conditions (FIG. 8C). This result suggested that there is no trade-off or a reduction in the LeX levels and rather the increase in sLeX levels could be attributed to enhanced biosynthesis.

The reversibility of the effect of 1b on increased sLeX levels was also investigated. Time course studies revealed that incubation of HL-60 cells with a single dosage of 1b or 1g (50 μM) resulted in peak sLeX expression on day 2 and returned to normal levels by day 6. There was a maximum of 3.0-fold and 2.2-fold increase in sLeX levels upon treatment, respectively, with 1b and 1g on day 2 while no change was observed in cells treated with controls either 1d or vehicle (D). By day 6 it was noticed that cells treated with 1g showed a slightly faster recovery to native levels while 1b treatment still maintained approximately 2.3-fold higher levels of sLeX (FIG. 8C). These results confirmed the advantages of pharmacological approaches, with dosage control and reversibility, over genetic methods for modulation of sLeX.

In addition to investigating the sLeX levels on surface of intact cells by flow cytometry, the sLeX epitopes by western blotting was studied. HL-60 cells were incubated with 1a, 1b, 1d, 1e, 1f, or 1g (50 μM) along with untreated (U) and vehicle-treated (D) controls, for 72 h and lyzed. Total lysates were resolved by SDS-PAGE, blotted on to nitrocellulose membranes, probed using anti-sLeX (CSLEX1) antibody followed by AF488-conjugated secondary antibody, and imaged using a fluorescence scanner. Robust increase in sLeX bands were observed between 150-100 kDa ranges in cells treated with 1b, with moderate increase with 1f and 1g, faint increase with 1a and 1e. Cells treated with controls (U, D, or 1d) were negative for sLeX epitopes (FIG. 8D). Consistent with flow cytometry results, the N-cyclobutanoyl analogue compound 1b showed maximum gain in sLeX levels compared to the corresponding straight chain N-pentanoyl analogue 1g. In contrast, the N-cyclopropanoyl analogue 1a showed lower levels of sLeX compared to its straight chain N-butanoyl analogue 1f. The western blotting data showed the interesting differential property of the N-cyclobutanoyl analogue compound 1b, which was not captured by the N-cyclopropanoyl compound 1a, thus highlighting the significance of ManNAc analogue-structure dependent biological outcomes.

Low levels of sLeX epitopes in HL-60 cells (both untreated and vehicle treated) upon western blotting with CSLEX1 antibody is consistent with the fact that HL-60 tend to spontaneously differentiate to hyposialylated phenotype during culture. Also, sLeX epitopes were not detectable upon treatment with either 1d or 1e (50 μM, 72 h), notwithstanding the two-fold increase in total sialic acid levels. Earlier work has shown that HL-60 cells showed enhanced expression of sLeX epitopes (probed with KM93 antibody), particularly on P-selectin glycoprotein ligand-1 (PSGL-1/CD162) upon treatment with the free monosaccharide ManNProp at 10 mM. No such effect was observed with its peracetylated counterpart 1e at 50 μM. The sLeX epitopes were observed to be increased moderately for both 1f and 1g, slightly for 1a, and maximally for 1b. The N-butanoyl analogue 1f has been shown to be an efficient inhibitor of polysialic acid attachment to neural cell adhesion molecules (NCAM). However, if showed an increase in sLeX levels in HL-60 cells while the corresponding N-cyclopropanoyl analogue 1a showed only a faint increase. The N-pentanoyl analogue compound 1g has been shown to alter contact dependent inhibition of cell growth and modulate viral binding to cell surface. It was found that treatment with 1g enhanced expression of sLeX. However, the corresponding five-carbon N-cyclobutanoyl analogue compound 1b showed the maximum increase in CSLEX1 epitopes (FIG. 8D). The observed increase in sLeX levels upon treatment with compound 1b could arise due to two plausible reasons, viz., (i) enhanced biosynthesis of both wild-type and modified sLeX carrying N-cyclobutanoyl-D-neuraminic acid (NeuCb) and (ii) enhanced binding affinity of CSLEX1 antibody to modified sLeX. Many glycoproteins, including CD162/PSGL-1, CD43, CD44, and ESL-1, have been reported to carry sLeX. The paratopes of CSLEX1 have not been well defined and it is possible that the N-cyclobutanoyl moiety of NeuCb makes direct contacts with hydrophobic residues on the antibody or alters the preferred conformations of sLeX for CSLEX1 binding.

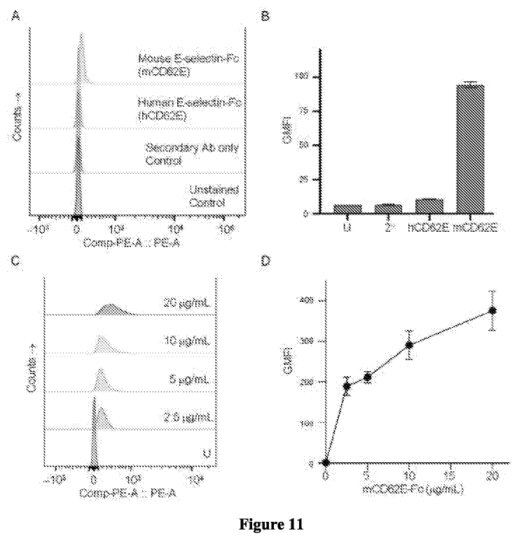

The essentiality of sLeX epitopes on leukocytes and their interaction with selectins (endothelial (E), leukocyte (L), and platelet (P); CD62E/L/P) for cell-cell communication, cellular migration, and extravasation through the high endothelial venules was investigated. Since treatment with compound 1b resulted in a robust enhancement of sLeX, as revealed by CSLEX1 binding, the effects on the binding to the endogenous receptor for sLeX, viz., E-selectin (CD62E) was tested. Binding of both mouse and human E-selectin-Fc chimera protein to HL-60 cells were studied using flow cytometry and conditions for saturation binding were optimized (FIG. 11). Mouse E-selectinFc (mCD62E) was found to show higher binding to HL-60 cells compared to human E-selectin-Fc (hCD62E). Flow cytometry results revealed a robust and significance increase of 2.3-fold in cells treated with 1b (50 μM, 72 h) with respect to untreated cells, similar to the results obtained for CSLEX1 binding (FIG. 8E). The controls treated with vehicle (D), 1d, or 1a showed no significant change with respect to untreated cells.

Thorough structural and kinetic binding studies have established that E-selectin binding to sLeX is determined by the carboxylate moiety of sialic acid and 3-OH/4-OH of the fucosyl moiety with the N-acetyl moiety projecting out to the solvent front and not having any direct contact. These aspects were exploited for the development of the minimal pharmacophore and potential inhibitors of sLeX interactions with E-selectin. It was observed that the compound 1b, which is processed through the sialic acid pathway, resulted in enhanced binding of both mCD62E as well as CSLEX1 antibody. Two possible reasons could explain the observed phenomenon with 1b, viz., (i) the N-cyclobutanoyl moiety does not make direct contacts with ligand biding pocket of E-selectin but might strongly influence and enhance the preferred conformations and (ii) the N-cyclobutanoyl moiety changes the binding drastically through direct contacts in E-selectin with hydrophobic pockets in the vicinity. Notably, the mCD62E showed higher binding to HL-60 cells compared to hCD62E which could be attributed to the expression of N-glycolyl-D-neuraminic acid (NeuGc) carrying sLeX in cells cultured in fetal bovine serum. This indicated that modifications to the N-acyl side chain, from N-acetyl to N-glycolyl could have profound effects on shifting conformation population dynamics to favorable binding. Similar effects might be operating for NeuCb carrying sLeX upon metabolic processing of 1b.

Cutaneous lymphocyte antigens (CLA) carry sLeX glycans and interact with E-selectin and enable T-cell migration. Anti-CLA antibody (HECA452) is a non-function blocking antibody which binds to sLeX epitopes. Effect of ManNAc analogues treatment on HL-60 cells for HECA452 binding through flow cytometry was tested. A prominent 40% decrease in HECA-452 binding was observed upon treatment with 1a or 1b (50 μM, 24 h) compared to untreated cells; while cells treated with vehicle (D) or 1d showed only a moderate increase (FIG. 8F). While a robust increase was found for binding of mCD62E and CSLEX1, a decrease in HECA452 was observed. It is known that HECA452 is specific for human sLeX carrying NeuAc and does not bind to mouse sLeX carrying NeuGc, suggesting the direct relevance of the N-acyl moiety for binding. In the case of treatment with 1a or 1b, it is possible that the HECA452 binding is abrogated, respectively, due to presence of NeuCp or NeuCb on sLeX. Additionally, increased sLeX biosynthesis, as a consequence of treatment with 1a or 1b, could in turn affect both glycan site occupancy and glycan densities on the glycoconjugates presenting paratopes for the HECA-452 antibody. Since HECA-452 is a non-function blocking antibody, it might depend on additional peptide backbone residues for contact which could be in principle masked by enhanced glycan site occupancy and the presence of engineered sLeX.

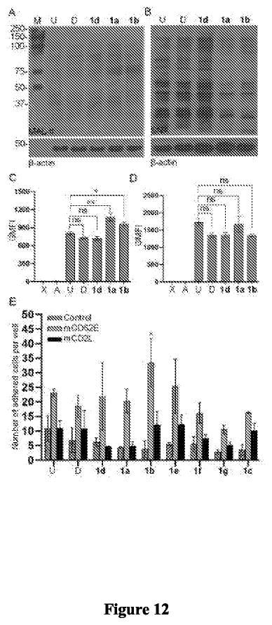

Apart from sLeX, the metabolic processing of ManNAc analogues could in principle affect various other types of sialoglycans on N- and O-linked glycoproteins as well as gangliosides. Global sialylation levels using sialic acid binding lectins, viz., Maackia amurensis lectin (MAL-II) and Sambucus nigra agglutinin (SNA) which bind, respectively, to NeuAca2→3Gal and NeuAca2→6Gal/GalNAc epitopes. HL-60 cells incubated with vehicle (D), 1a, 1b, or 1d (50 μM, 72 h) were lysed, resolved on SDS-PAGE, transferred to nitrocellulose membranes and probed with biotinylated MAL-II or biotinylated SNA followed by horse radish peroxidase conjugated avidin (HRP-avidin). MAL-II epitopes in control cells (U, D and 1d) were found to be below detection limit with a slight increase noted upon treatment with 1a or 1b (FIG. 12A). MAL-II blot results were consistent with earlier observations of spontaneously induced hypo-sialylated phenotype of HL-60 cells in culture. By contrast, treatment of 1a or 1b resulted in significant reduction of SNA epitopes compared to controls (U, D, or 1d) (FIG. 12B). A comparison of lectin blots of cells (U, D, and 1d) reveal striking differential of abundant SNA epitopes compared to low levels of MAL-II epitopes, suggesting differential endogenous activities of α2→3 vs. α2→6 sialyl transferases. Further evaluation of lectin binding on intact cells by flow cytometry confirmed significant gain in MAL-II binding upon treatment with 1a or 1b, but not in controls, compared to untreated cells (FIG. 12C); no significant differences were observed for SNA binding upon treatment with D, 1d, 1a, or 1b compared to untreated cells (FIG. 12D). The increase in MAL-II with a concomitant decrease in SNA epitopes upon treatment with 1b is in agreement with the enhanced binding observed for CSLEX1 and E-selectin-Fc since the sLeX contains the NeuAca2->3Gal moiety.

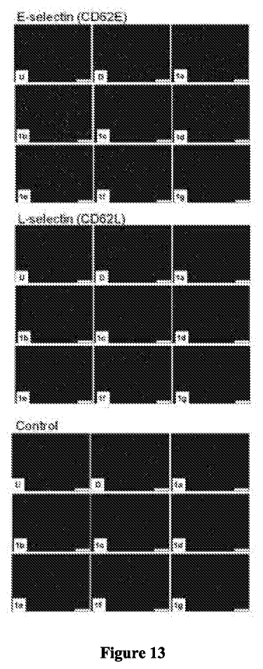

The compound 1b was found to be exhibiting enhanced expression of sLeX, and further functional studies on cell adhesion was studies. Cell adhesion studies were performed on plastic plates coated with protein-G followed by either mouse E-selectin-Fc (mCD62E) chimera or mouse L-selectin-Fc (mCD62L) chimera, followed by blocking with BSA. Plates coated with only protein-G and BSA were used as controls. L-selectin was selected in order to study the effect of ManNAc analogues on sulfo-sialyl-Lewis-X (sulfo-sLeX) expression. HL-60 cells were incubated with the ManNAc analogues 1a-1g (50 μM, 48 h), along with untreated and vehicle controls, harvested, and allowed to adhere. After 60 min, the cells in suspension were removed and the surface-bound cells were fixed, stained with DAPI, imaged by microscopy, and the numbers of cells were enumerated (FIG. 13 and FIG. 12E). HL-60 cells pre-incubated with the compound 1b exhibited about 40% increase in the numbers of adhered cells on mCD62E coated surface compared to all other conditions. Reduced numbers of cells were found to adhere to mCD62L coated surfaces under all the conditions compared to untreated controls; however, no differential effects were observed with respect to Protein-G and BSA only coated surfaces. These results established that treatment with compound 1b, but not controls, resulted in increased binding to CSLEX1, mCD62E, and MAL-II, and in turn exhibited improved adherent properties to E-selectin coated surfaces in a static adhesion model.

### Example 7

### Compound 1b

The present disclosure provides compounds of Formula I, II, III and IV as disclosed herein and it can be found that the compound 1b or MA-3 (3S,4R,5S,6R)-6-(acetoxymethyl)-3-(cyclobutanecarboxamido)tetrahydro-2H-pyran-2,4,5-triyl triacetate, the cyclobutanoyl derivative ManNAc analogue was found to be efficient in modulating cell-cell interactions, glycan-protein interactions, glycan-glycan interactions, sialic acids-proteins interactions, plant-pathogen interactions, sialyl-Lewis X/Lewis Y-selectins interactions, sialoglycoconjugates-siglecs interactions, polysialic acid-sialo-lectins interactions, sialic acid-neuraminidases interactions, sialic acid-hemagglutinin interactions, sialic acid-microbial adhesin interactions, or combinations thereof. The compound 1b results in increased flux of sialic acid biosynthesis compared to the corresponding linear chain derivative of the ManNac analogues. This is because the steric effect play a significant role in the sialic acid biosynthesis and thereby the structure of the compound 1b was found to be more favourable. Further the compound 1b showed a maximum gain in sLex levels. N-cyclobutanoyl ManNAc compound 1b, efficiently enhanced sLeX levels in an analogue structure-dependent manner and lead to enhanced adhesion to E-selectin. ManNAc analogues and multiple complementary approaches have shown the analogue-structure dependent effects on sLeX expression and consequential modulation of their binding to anti-glycan antibodies, glycan-binding proteins, and lectins. Therefore ability to either enhance or suppress sLeX-selectin, sialoglycans-siglec, and sialoglycans-viral hemagglutinin interactions using pharmacological agents has the potential to open up new avenues for future therapeutics for immune deficiency disorders as well as shed light on the intricate mechanisms of functional roles of glycans in immune regulation.

### Advantages of the Present Invention

The present invention provides compounds of Formula I and a process for preparing the same. The present invention also provides a pharmaceutical composition comprising the compounds of the Formula I. The compounds of the present invention are small molecules that are capable of modulating cell-cell interactions, cell-pathogen and cell-extracellular matrix interactions. The compounds of Formula I can modulate various interactions such as glycan-protein interactions, glycan-glycan interactions, sialic acids-proteinsinteractions, plant-pathogen interactions, sialyl-Lewis X/Lewis Y-selectinsinteractions, sialoglycoconjugates-siglecs interactions, polysialic acid-sialo-lectins interactions, sialic acid-neuraminidases interactions, sialic acid-hemagglutinin interactions, and sialic acid-microbial adhesin interactions. The compounds of the present invention modifies biosynthetic pathways either directly or through metabolic processing. The compounds of the present invention modulate processes of cellular migration including extravasation, intravasation, leukocyte homing, and cancer cell metastasis. These compounds act as adjuncts in combinatorial cancer chemotherapy and in combinatorial cancer immunotherapy. The compounds of Formula I of the present invention can be used in engineering of cell surface epitopes such as sialic acid epitope, sialyl-Lewis-A epitope, sialyl-Lewis-X epitope, sulfo-sialyl-Lewis-X epitope, Lewis-X(CD15)/Y/A/B epitope, Sialyl-TF (Thomson-Friedenreich) antigen, gangliosides, or polysialicacid epitopes. The compounds of Formula I is also a potential candidate to modulate plant glycosylation and to alter plant-pathogen interactions. Further the compounds of Formula I can modulate mammalian bioreactor cultures, fungus, and yeast systems for production of glycan-modified biologics and biopharmaceuticals. Overall, the compounds of the present invention are small molecules which can exhibit various pharmacological activities for the modification or prevention or treatment of various disorders or conditions or diseases.

I/We claim:

1. A compound of Formula I 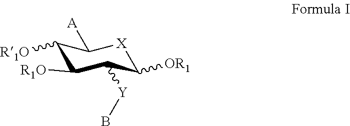
wherein A is C₁₋₁₂ alkyl, or —C₁₋₁₂ alkyl OR₁; wherein R₁ and R′₁ is independently selected from hydrogen, —C(O)C₁₋₁₂ alkyl, —C(O)C₂₋₁₂ alkenyl, or —C(O)C₂₋₁₂ alkynyl;
X is selected from O, S, Se, —CH₂, or —C(OH)R₂;
Y is —NR₂, or —CHR₂;
B is selected from hydrogen, —C(═Z)—(CH₂)ₘ—C₃₋₁₂ cycloalkyl, or —C(═Z)—(CH₂)ₘ—C₁₋₁₂ heterocyclyl;
Z is O, or CH₂;
R₂ is selected from hydrogen, C₁₋₁₂ alkyl, C₂₋₁₂ alkenyl, C₂₋₁₂ alkynyl, or C₆₋₁₄aryl;
m is 0 to 4;
provided when —(OR′₁) is equatorial, —(Y—B) is either axial or equatorial,
and m=0, then B≠C(═Z)—(CH₂)ₘ—C₃ cycloalkyl.

2. The compound as claimed in claim 1, wherein R₁ and R′₁ is independently selected from hydrogen, or —C(O)C₁₋₃ alkyl;
X is O, or —CH₂;
Y is —NR₂;
B is hydrogen, or —C(═Z)—(CH₂)ₘ—C₃₋₁₂ cycloalkyl;
Z is O, or CH₂;
R₂ is hydrogen, or C₁₋₈ alkyl;
m is 0 or 1;
n is 0 to 7;
provided when —(OR′₁) is equatorial, —(Y—B) is either axial or equatorial,
and m=0, then B≠C(═Z)—(CH₂)ₘ—C₃ cycloalkyl.

3. A compound of 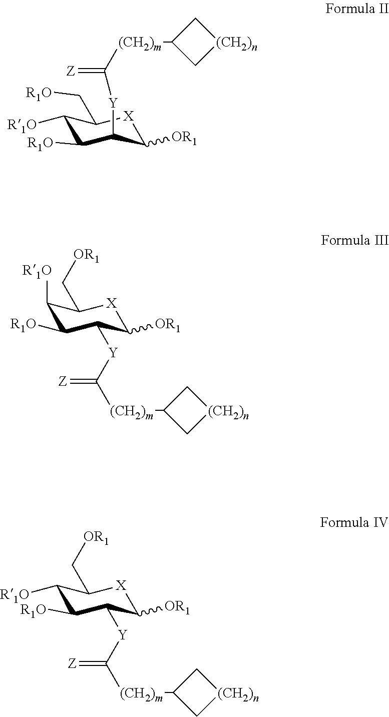
wherein R₁ and R′₁ is independently selected from hydrogen, or —C(O)C₁₋₃ alkyl; X is O; Y is —NR₂; Z is O; R₂ is hydrogen, or C₁₋₈ alkyl; m is 0 or 1,
n is 0 to 7;
when m of Formula II is 0, then n is 1 to 7;
when m of Formula IV is 0, then n is 1 to 7;
when m of Formula II is 1, then n is 0 to 7; and
when m of Formula IV is 1, then n is 0 to 7.

4. The compound as claimed in any one of the claims 1-3, wherein the compound is selected from
i. (3S,4R,5S,6R)-6-(acetoxymethyl)-3-(cyclobutanecarboxamido)tetrahydro-2H-pyran-2,4,5-triyl triacetate/Acetyl 3,4,6-tri-O-acetyl-2-cyclobutanecarboxamido-2-deoxy-α/β-D-manno-hexopyranoside
ii. (3S,4R,5S,6R)-6-(acetoxymethyl)-3-(cyclopentanecarboxamido)tetrahydro-2H-pyran-2,4,5-triyl triacetate/Acetyl 3,4,6-tri-O-acetyl-2-cyclopentanecarboxamido-2-deoxy-α/β-D-manno-hexopyranoside
iii. (3S,4R,5S,6R)-6-(acetoxymethyl)-3-(cyclohexanecarboxamido)tetrahydro-2H-pyran-2,4,5-triyl triacetate/Acetyl 3,4,6-tri-O-acetyl-2-cyclohexanecarboxamido-2-deoxy-α/β-D-manno-hexopyranoside
iv. (3S,4R,5S,6R)-6-(acetoxymethyl)-3-(cycloheptanecarboxamido)tetrahydro-2H-pyran-2,4,5-triyl triacetate/Acetyl 3,4,6-tri-O-acetyl-2-cycloheptanecarboxamido-2-deoxy-α/β-D-manno-hexopyranoside
v. (3R,4R,5R,6R)-6-(acetoxymethyl)-3-(cyclopropanecarboxamido)tetrahydro-2H-pyran-2,4,5-triyl triacetate/Acetyl 3,4,6-tri-O-acetyl-2-cyclopropanecarboxamido-2-deoxy-α/β-D-galacto-hexopyranoside
vi. (3R,4R,5R,6R)-6-(acetoxymethyl)-3-(cyclobutanecarboxamido)tetrahydro-2H-pyran-2,4,5-triyl triacetate/Acetyl 3,4,6-tri-O-acetyl-2-cyclobutanecarboxamido-2-deoxy-α/β-D-galacto-hexopyranoside
vii. (3R,4R,5R,6R)-6-(acetoxymethyl)-3-(cyclopentanecarboxamido)tetrahydro-2H-pyran-2,4,5-triyl triacetate/Acetyl 3,4,6-tri-O-acetyl-2-cyclopentanecarboxamido-2-deoxy-α/β-D-galacto-hexopyranoside
viii. (3R,4R,5R,6R)-6-(acetoxymethyl)-3-(cyclohexanecarboxamido)tetrahydro-2H-pyran-2,4,5-triyl triacetate/Acetyl 3,4,6-tri-O-acetyl-2-cyclohexanecarboxamido-2-deoxy-α/β-D-galacto-hexopyranoside
ix. (3R,4R,5R,6R)-6-(acetoxymethyl)-3-(cycloheptanecarboxamido)tetrahydro-2H-pyran-2,4,5-triyl triacetate/Acetyl 3,4,6-tri-O-acetyl-2-cycloheptanecarboxamido-2-deoxy-α/β-D-galacto-hexopyranoside
x. (3R,4R,5S,6R)-6-(acetoxymethyl)-3-(cyclobutanecarboxamido)tetrahydro-2H-pyran-2,4,5-triyl triacetate/Acetyl 3,4,6-tri-O-acetyl-2-cyclobutanecarboxamido-2-deoxy-α/β-D-gluco-hexopyranoside
xi. (3R,4R,5S,6R)-6-(acetoxymethyl)-3-(cyclopentanecarboxamido)tetrahydro-2H-pyran-2,4,5-triyl triacetate/Acetyl 3,4,6-tri-O-acetyl-2-cyclopentanecarboxamido-2-deoxy-α/β-D-gluco-hexopyranoside
xii. (3R,4R,5S,6R)-6-(acetoxymethyl)-3-(cyclohexanecarboxamido)tetrahydro-2H-pyran-2,4,5-triyl triacetate/Acetyl 3,4,6-tri-O-acetyl-2-cyclohexanecarboxamido-2-deoxy-α/β-D-gluco-hexopyranoside
xiii. (3R,4R,5S,6R)-6-(acetoxymethyl)-3-(cycloheptanecarboxamido)tetrahydro-2H-pyran-2,4,5-triyl triacetate/Acetyl 3,4,6-tri-O-acetyl-2-cycloheptanecarboxamido-2-deoxy-α/β-D-gluco-hexopyranoside
xiv. (3S,4R,5S,6R)-6-(acetoxymethyl)-3-(2-cyclopropylacetamido)tetrahydro-2H-pyran-2,4,5-triyl triacetate/Acetyl 3,4,6-tri-O-acetyl-2-cyclopropylacetamido-2-deoxy-α/β-D-manno-hexopyranoside
xv. (3S,4R,5S,6R)-6-(acetoxymethyl)-3-(2-cyclobutylacetamido)tetrahydro-2H-pyran-2,4,5-triyl triacetate/Acetyl 3,4,6-tri-O-acetyl-2-cyclobutylacetamido-2-deoxy-α/β-D-manno-hexopyranoside
xvi. (3S,4R,5S,6R)-6-(acetoxymethyl)-3-(2-cyclopentylacetamido)tetrahydro-2H-pyran-2,4,5-triyl triacetate/Acetyl 3,4,6-tri-O-acetyl-2-cyclopentylacetamido-2-deoxy-α/β-D-manno-hexopyranoside
xvii. (3S,4R,5S,6R)-6-(acetoxymethyl)-3-(2-cyclohexylacetamido)tetrahydro-2H-pyran-2,4,5-triyl triacetate/Acetyl 3,4,6-tri-O-acetyl-2-cyclohexylacetamido-2-deoxy-α/β-D-manno-hexopyranoside
xviii. (3S,4R,5S,6R)-6-(acetoxymethyl)-3-(2-cycloheptylacetamido)tetrahydro-2H-pyran-2,4,5-triyl triacetate/Acetyl 3,4,6-tri-O-acetyl-2-cycloheptylacetamido-2-deoxy-α/β-D-manno-hexopyranoside
xix. (3R,4R,5R,6R)-6-(acetoxymethyl)-3-(2-cyclopropylacetamido)tetrahydro-2H-pyran-2,4,5-triyl triacetate/Acetyl 3,4,6-tri-O-acetyl-2-cyclopropylacetamido-2-deoxy-α/β-D-galacto-hexopyranoside
xx. (3R,4R,5R,6R)-6-(acetoxymethyl)-3-(2-cyclobutylacetamido)tetrahydro-2H-pyran-2,4,5-triyl triacetate/Acetyl 3,4,6-tri-O-acetyl-2-cyclobutylacetamido-2-deoxy-α/β-D-galacto-hexopyranoside
xxi. (3R,4R,5R,6R)-6-(acetoxymethyl)-3-(2-cyclopentylacetamido)tetrahydro-2H-pyran-2,4,5-triyl triacetate/Acetyl 3,4,6-tri-O-acetyl-2-cyclopentylacetamido-2-deoxy-α/β-D-galacto-hexopyranoside
xxii. (3R,4R,5R,6R)-6-(acetoxymethyl)-3-(2-cyclohexylacetamido)tetrahydro-2H-pyran-2,4,5-triyl triacetate/Acetyl 3,4,6-tri-O-acetyl-2-cyclohexylacetamido-2-deoxy-α/β-D-galacto-hexopyranoside
xxiii. (3R,4R,5R,6R)-6-(acetoxymethyl)-3-(2-cycloheptylacetamido)tetrahydro-2H-pyran-2,4,5-triyl triacetate/Acetyl 3,4,6-tri-O-acetyl-2-cycloheptylacetamido-2-deoxy-α/β-D-galacto-hexopyranoside
xxiv. (3R,4R,5S,6R)-6-(acetoxymethyl)-3-(2-cyclopropylacetamido)tetrahydro-2H-pyran-2,4,5-triyl triacetate/Acetyl 3,4,6-tri-O-acetyl-2-cyclopropylacetamido-2-deoxy-α/β-D-gluco-hexopyranoside;
xxv. (3R,4R,5S,6R)-6-(acetoxymethyl)-3-(2-cyclobutylacetamido)tetrahydro-2H-pyran-2,4,5-triyl triacetate/Acetyl 3,4,6-tri-O-acetyl-2-cyclobutylacetamido-2-deoxy-α/β-D-gluco-hexopyranoside
xxvi. (3R,4R,5S,6R)-6-(acetoxymethyl)-3-(2-cyclopentylacetamido)tetrahydro-2H-pyran-2,4,5-triyl triacetate/Acetyl 3,4,6-tri-O-acetyl-2-cyclopentylacetamido-2-deoxy-α/β-D-gluco-hexopyranoside
xxvii. (3R,4R,5S,6R)-6-(acetoxymethyl)-3-(2-cyclohexylacetamido)tetrahydro-2H-pyran-2,4,5-triyl triacetate/Acetyl 3,4,6-tri-O-acetyl-2-cyclohexylacetamido-2-deoxy-α/β-D-gluco-hexopyranoside
xxviii. (3R,4R,5S,6R)-6-(acetoxymethyl)-3-(2-cycloheptylacetamido)tetrahydro-2H-pyran-2,4,5-triyl triacetate/Acetyl 3,4,6-tri-O-acetyl-2-cycloheptylacetamido-2-deoxy-α/β-D-gluco-hexopyranoside

5. A process for preparing the compound as claimed in any one of the claims 1 to 4, the process comprising reacting at least one selectively and orthogonally protected hexosamine salt of Formula A, with at least one carboxylic acid of Formula R₂CO₂H to obtain the compound of Formula I, 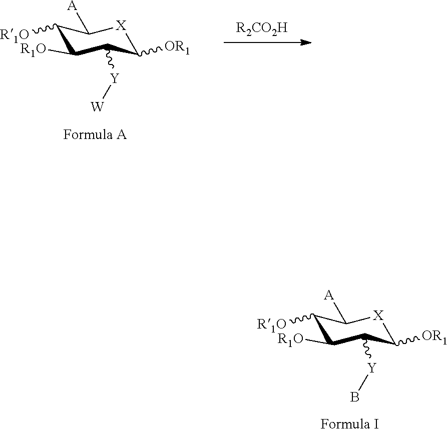
wherein R₂ is selected from C₃₋₁₂ cycloalkyl-(CH₂)ₘ—, C₁₋₁₂ heterocyclyl-(CH₂)ₘ, or C₂₋₂₄ alkyl heterocyclyl)-(CH₂)ₘ—;
W is selected from hydrogen, or C₁₋₁₂ alkyl;
A is C₁₋₁₂ alkyl, or —C₁₋₁₂ alkyl OR₁;
R₁ and R′₁ is independently selected from hydrogen —C(O)C₂₋₁₂ alkenyl, or —C(O)C₂₋₁₂ alkynyl;
X is selected from O, S, Se, —CH₂, or —C(OH)R₂,
Y is —NR₂, or —CHR₂;
B is selected from hydrogen, —C(═Z)—(CH₂)ₘ—C₃₋₁₂ cycloalkyl, or —C(═Z)—(CH₂)ₘ—C₁₋₁₂ heterocyclyl;
Z is O, or CH₂;
R₂ is selected from hydrogen, C₁₋₁₂ alkyl, C₂₋₁₂ alkenyl, C₂₋₁₂ alkynyl, or C₆₋₁₄aryl;
m is 0 to 7;
provided when —(OR′₁) is equatorial, —(Y—B) is either axial or equatorial,
and m=0, then B≠C(═Z)—(CH₂)ₘ—C₃ cycloalkyl.

6. The process as claimed in claim 5, wherein the process is carried out in the presence of a coupling agent, a base, a solvent, or combinations thereof at a temperature in a range of 0° C. to 100° C.

7. The process as claimed in claim 6, wherein the coupling agent is selected from 1-ethyl-3-(3-dimethylaminopropyl)carbodiimide hydrochloride, dicyclohexyl carbodiimide, diisopropylcarbodiimide, hexafluorophosphate azabenzotriazoletetramethyluronium, 1,1′-carbonyldiimidazole, 1-hydroxybenzotriazole, or combinations thereof; the base is selected from pyridine, triethylamine, 4-(N,N-dimethylamino)pyridine, sodium bi-carbonate, sodium carbonate, lithium carbonate, ammonium bicarbonate, or combinations thereof; and the solvent is selected from dimethyl formamide, dioxan, dichloromethane, chloroform, acetonitrile, ethyleneglycol, tetrahydrofuran, cyclohexane, or combinations thereof.

8. The compound as claimed in any one of the claims 1 to 4, wherein the compound of Formula-I has a glycomimetic structure, both directly and via metabolic processing, for modifying sialoglycans, mucin-type O-glycans (MTOG), selectins, siglecs, β-O-GlcNAc-ylation, or combinations thereof.

9. The compound as claimed in any one of the claims 1 to 4, wherein the compound of Formula I modifies cell-cell interactions, cell-pathogen interactions, or cell-extracellular matrix interactions.

10. The compound as claimed in any one of the claims 1 to 4, wherein the compound of Formula I is capable of modifying glycan-protein interactions, glycan-glycan interactions, sialic acids-proteins interactions, plant-pathogen interactions, sialyl-Lewis X/Lewis Y-selectins interactions, sialoglycoconjugates-siglecs interactions, polysialic acid-sialo-lectins interactions, sialic acid-neuraminidases interactions, sialic acid-hemagglutinin interactions, sialic acid-microbial adhesin interactions, or combinations thereof.

11. The compound as claimed in claim 1, wherein the compound is an adjunct in combinatorial cancer chemotherapy or an adjunct in combinatory cancer immunotherapy.

12. A pharmaceutical composition comprising the compound of Formula I as claimed in any one of the claims 1 to 4 optionally with at least one pharmaceutically acceptable salt thereof.

13. A compound as claimed in claim 1, capable of modifying cell-cell interactions, cell-pathogen interactions, or cell-extracellular matrix interactions, glycan-protein interactions, glycan-glycan interactions, sialic acids-proteins interactions, plant-pathogen interactions, sialyl-Lewis X/Lewis Y-selectins interactions, sialoglycoconjugates-siglecs interactions, polysialic acid-sialo-lectins interactions, sialic acid-neuraminidases interactions, sialic acid-hemagglutinin interactions, sialic acid-microbial adhesin interactions, or combinations thereof, wherein the compound is compound 1

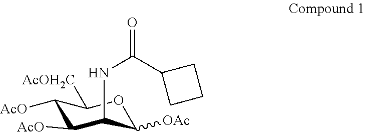

14. A pharmaceutical composition comprising the compound 1 as claimed in claim 13, optionally with at least one pharmaceutically acceptable excipient thereof.

15. A method of prevention or treatment or modification of a condition or a disease, the method comprising administering the compound of Formula I as claimed in claim 1 or the pharmaceutical composition as claimed in claim 12 to a subject in need thereof.

16. The method as claimed in claim 15, wherein the condition or the disease is selected from acute inflammatory disorders, chronic inflammatory disorders, asthma, allergy, psoriasis, rheumatoid arthritis, tumor metastasis, cancer metastasis, reperfusion syndrome, cytokine storm, viral infectious diseases, bacterial infectious diseases, acute inflammation of liver, neutrophil infiltration, xenograft maintenance, neoplasm, tuberculosis, acute liver injury, acute kidney injury, thrombosis, chronic obstructive pulmonary disease (COPD), hay fever, stroke, atherosclerosis, rhinitis, contact dermatitis, atopic dermatitis, inflammatory bowel disease, multiple sclerosis, type I diabetes, organ transplant rejection, systemic lupus erythematosus (SLE), cystic fibrosis, congenital disorders of glycosylation, leukocyte adhesion deficiency disorder, or fertilization disorder.

17. A method for engineering cell surface epitopes, the method comprising: contacting a cell with the compound of Formula I as claimed in claim 1 or the pharmaceutical composition as claimed in claim 12.

18. The method as claimed in claim 17, wherein the method is carried out optionally through metabolic biosynthetic pathways.

19. The method as claimed in claim 17, wherein the cell surface epitope is glycan epitope selected from sialic acid epitope, sialyl-Lewis-A epitope, sialyl-Lewis-X epitope, sulfo-sialyl-Lewis-X epitope, Lewis-X(CD15)/Y/A/B epitope, Sialyl-TF (Thomson-Friedenreich) antigen, gangliosides, or polysialicacid epitopes.

20. Use of the compound as claimed in claim 1 or the pharmaceutical composition as claimed in claim 12, for prevention or treatment or modification of a condition or a disease selected from acute inflammatory disorders, chronic inflammatory disorders, asthma, allergy, psoriasis, rheumatoid arthritis, tumor metastasis, cancer metastasis, reperfusion syndrome, cytokine storm, viral infectious diseases, bacterial infectious diseases, acute inflammation of liver, neutrophil infiltration, xenograft maintenance, neoplasm, tuberculosis, acute liver injury, acute kidney injury, thrombosis, chronic obstructive pulmonary disease (COPD), hay fever, stroke, atherosclerosis, rhinitis, contact dermatitis, atopic dermatitis, inflammatory bowel disease, multiple sclerosis, type I diabetes, organ transplant rejection, systemic lupus erythematosus (SLE), cystic fibrosis, congenital disorders of glycosylation, leukocyte adhesion deficiency disorder, or fertilization disorder.

21. Use of the compound as claimed in claim 1 or the pharmaceutical composition as claimed in claim 12, for engineering cell surface epitopes by contacting a cell with the compound of Formula I as claimed in claim 1 or the pharmaceutical composition as claimed in claim 12, wherein the cell surface epitope is glycan epitope selected from sialic acid epitope, sialyl-Lewis-A epitope, sialyl-Lewis-X epitope, sulfo-sialyl-Lewis-X epitope, Lewis-X(CD15)/Y/A/B epitope, Sialyl-TF (Thomson-Friedenreich) antigen, gangliosides, or polysialicacid epitopes.

22. The use as claimed in claim 21, wherein contacting a cell is carried out optionally through metabolic biosynthetic pathways.
